(19) United States
(12) Patent Application Publication
(10) Pub. No.: US 2025/0072793 A1
(43) Pub. Date: Mar. 6, 2025

(54) System and Method for Identifying Clinically-Similar Clusters of Daily Continuous Glucose Monitoring (CGM) Profiles

(71) Applicant: UNIVERSITY OF VIRGINIA PATENT FOUNDATION, Charlottesville, VA (US)

(72) Inventors: Boris P. KOVATCHEV, Charlottesville, VA (US); Benjamin J. LOBO, Charlottesville, VA (US)

(73) Assignee: UNIVERSITY OF VIRGINIA PATENT FOUNDATION, Charlottesville, VA (US)

(21) Appl. No.: 18/710,811

(22) PCT Filed: Apr. 26, 2023
(86) PCT No.: PCT/US2023/020014

§ 371 Date: May 16, 2024

Related U.S. Application Data
(60) Provisional application No. 63/335,361, filed on Apr. 27, 2022.
(60) Provisional application No. 63/443,918, filed on Feb. 7, 2023.

(51) Int. Cl. A61B 5/145; G16H 20/10; G16H 50/20; G16H 50/70

(52) U.S. Cl. CPC A61B 5/14532; G16H 20/10; G16H 50/20; G16H 50/70

(57) ABSTRACT

Embodiments relate to a system for processing glucose data by efficient glucose database management. The system includes a physical data store containing glucose measurement data and a representation for at least one cluster of the glucose measurement data, wherein the representation approximates a glycemic profile vector array for a cluster of multiple glucose profiles segmented by plural time ranges. The system includes a processor and computer memory configured with instructions stored thereon that when executed will cause the processor to: 1) receive glucose measurements; 2) convert the glucose measurements into vectorial form; 3) search the physical data store by comparing a newly received glucose measurement to a centroid of a cluster using a similarity metric; 3) classify the newly received glucose measurement with a cluster having a matched similarity metric based on the comparing; and 4) ascribe a treatment to the newly received glucose measurement.

### CROSS-REFERENCE TO RELATED APPLICATIONS

This patent application is related to and claims the benefit of priority of U.S. Provisional Application No. 63/443,918, filed on Feb. 7, 2023, and U.S. Provisional Application No. 63/335,361, filed on Apr. 27, 2022, the entire contents of which each incorporated by reference.

### FIELD

Embodiments relate system for processing glucose data by efficient glucose database management and using classified glucose data to monitor, analyze, influence, etc. a concentration of glucose level in a fluid.

### BACKGROUND INFORMATION

Glucose variability (GV) in diabetes reflects an underlying bio-behavioral process of blood glucose (BG) fluctuation that has two principal dimensions: amplitude reflecting the extent of BG excursion, and time reflecting the frequency of BG variation and the rate of event progression. In the past 20 years, the ability to observe this process has evolved from episodic self-monitoring (e.g., a few BG determinations per day) to contemporary continuous glucose monitoring (CGM), which captures dense data sets of BG readings that are equally spaced in time (e.g., every 5 minutes). These data sets, known as time series, open new possibilities for the analysis and the optimal control of the human metabolic system in diabetes, including assessment of system dynamics, prediction of BG trends and events such as impending hypoglycemia or hyperglycemia, and automated closed-loop control commonly referred to as the “artificial pancreas”.

The widespread adoption of CGM technologies inevitably creates vast amounts of data; for example, our recent report of real-life use of an artificial pancreas system was based on over a billion data point. Diabetes data ecosystems play an increasingly important role supporting data sharing, virtual clinics, and remote access. Cloud databases accumulate these data and demand the use of data science tools, such as pattern recognition, neural networks, deep learning, and artificial intelligence, all of which can contribute to improvement of treatment and creation of fully-automated systems. A most promising application of Cloud databases and Data Science tools is the use of adaptation technologies that can “learn” and personalize the treatment to each individual. For this to work, appropriate structure needs to be created in the CGM data space, that represents the clinical meaning of the CGM data profiles well, and at the same time is simple, finite, and fixed, so the structure does not need to change with every new data set.

A number of glycemic control metrics exist, discussed in detail in a 2017 paper we published in Nature Reviews Endocrinology. CGM-based metrics should typically include some notion of the timing of CGM readings, not only of their amplitude. Some of the existing measures, such as MAGE (Mean amplitude of glucose excursions) and LBGI/HBGI (Low and High BG Indices) have been adapted for CGM use as well: the adaptation of MAGE for CGM data followed the classic time-independent structure of this measure, and therefore in this case CGM was only used as a source for amplitude assessment; the adaptation of the LBGI and the HBGI accounted for differences between SMBG and CGM data. The Mean of Daily Differences (MODD) was introduced as a measure of intra-day variability, and the Continuous Overlapping Net Glycemic Action (CONGA) was presented as a composite index of the magnitude and the timing of BG fluctuations captured over various time periods. The standard deviation of the BG rate of change was used as a marker of the stability of the metabolic system over time, based on the premise that more erratic BG changes are signs of system instability. An array of standard deviations was introduced to reflect GV contained within different clinically-relevant periods of CGM data and the clinical interpretation of various CGM-based metrics of glucose variability was discussed. A review of the statistical methods available for the analysis of CGM data included several graphs, such as Poincard plot of system stability, and the Variability-Grid Analysis (VGA) used to visualize glycemic fluctuations captured by CGM [12]. The VGA was also used to depict the efficacy of closed-loop control algorithms [4][13]. A perspective published in Diabetes Care re-evaluated several of the methods for computing and visualization of GV in the context of the relationship between GV and the risk for hypoglycemia, and we refer the reader to this paper for further details on the interpretation of the VGA and of the Poincard plot of CGM data.

Because the CGM field was overloaded not only by the voluminous and complex data sets, but also with a multitude of metrics used to assess various aspects of the CGM profiles, the 2019 International Consensus on Time in Range (TIR, typically 70-180 md/dL), to which we contributed, proposed TIR as a primary CGM-based metric of glycemic control and set clinical targets for its use. In the past 3 years, the “TIR system of metrics” received widespread adoption. The TIR system is based on the Ambulatory Glucose Profile (AGP), introduced as a template for data presentation and visualization. Originally developed by Mazze et. al., the standardized CGM report incorporates core CGM metrics and targets along with a 14-day composite glucose profile as an integral component of clinical decision making. This recommendation was endorsed by the international consensus and is also referenced by the American Diabetes Association 2019 Standards of Care and the AACE consensus on use of CGM. The AGP report is now adopted by most CGM device manufacturers in their CGM companion software. An example of the AGP report and the TIR system of metrics is presented in FIG. 2.

The TIR system of metrics defines 5 time in ranges for blood glucose values. These time in ranges are used in addition to the AGP to provide numerical interpretation of the AGP plot. In one embodiment, for example, these time in ranges are: Level 2 hypoglycemia—below 54 mg/dl, Level 1 hypoglycemia—from 54 to 69 mg/dL, within Target Range (TIR)—70 to 180 mg/dL, Level 1 hyperglycemia—from 180 to 250 mg/dL, and Level 2 hyperglycemia—above 250 mg/dL. Other embodiments of TIRs are presented in FIG. 3, according to the Consensus recommendations for different types of diabetes.

As seen in FIGS. 2 and 3, both the AGP and the TIR system of metrics do not represent inter-day variability of the CGM traces, and do not provide a fixed finite structure of the multitude of daily CGM profiles. An aspect of an embodiment of the present invention system, method, and computer readable medium takes this next step forward.

### SUMMARY

Embodiments can relate to a system for processing glucose data by efficient glucose database management. The system can include a physical data store containing glucose measurement data and a representation for at least one cluster of the glucose measurement data. The representation can approximate a glycemic profile vector array for a cluster of multiple glucose profiles segmented by plural time ranges. The system can include a processor and computer memory configured with instructions stored thereon that when executed will cause the processor to perform any of the method steps disclosed herein. Instructions can cause the processor to receive glucose measurements. Instructions can cause the processor to convert the glucose measurements into vectorial form. Instructions can cause the processor to search the physical data store by comparing a newly received glucose measurement to a centroid of a cluster using a similarity metric. Instructions can cause the processor to classify the newly received glucose measurement with a cluster having a matched similarity metric based on the comparing. Instructions can cause the processor to ascribe a treatment to the newly received glucose measurement.

Embodiments can relate to a method for processing glucose data for efficient glucose database management. The method can involve receiving glucose measurements. The method can involve converting the glucose measurements into vectorial form. The method can involve searching a physical data store by comparing a newly received glucose measurement to a centroid of a cluster using a similarity metric. The physical data store can contain glucose measurement data and a representation for at least one cluster of the glucose measurement data. The representation can approximate a glycemic profile vector for a cluster of multiple glucose profiles segmented by plural time ranges. The method can involve classifying the newly received glucose measurement with a cluster having a matched similarity metric based on the comparing. The method can involve ascribing a treatment to the newly received glucose measurement.

### BRIEF DESCRIPTION OF THE DRAWINGS

Other features and advantages of the present disclosure will become more apparent upon reading the following detailed description in conjunction with the accompanying drawings, wherein like elements are designated by like numerals, and wherein:

FIG. 1A is an exemplary system that can be used for processing glucose data by efficient glucose database management;

FIG. 1B is an exemplary system that can be used for developing a glucose database of clustered data sets;

FIG. 2 is an exemplary Ambulatory Glucose Profile with recommended time in ranges;

FIG. 3 are recommendations of the International Consensus on TIR displayed as CGM-based targets visualizations;

FIG. 4 shows an exemplary single iteration of a process that can be used to identify and evaluate a candidate set of CSCs;

FIG. 5 shows an exemplary CGM-based targets visualization for each of the 35 CSCs ordered by TIR.

FIG. 6 shows exemplary scatterplots of the points (dpᵢₖ, CSCₖ(dpi)) for kϵ{T54, T70, TIR, T180, T250} when using the daily CGM profiles from the Testing data set (but excluding the 1,169 daily CGM profiles from healthy individuals);

FIG. 7 shows exemplary individual (fᵢ), mean (f_{G}), and fitted traces stratified by health state and treatment modality;

FIG. 8 shows exemplary frequency and cumulative frequency distributions of the daily CGM profiles in the Testing data set to the 35 CSCs, stratified by health state and T1D treatment modality;

FIG. 9 shows exemplary boxplots of the CSC index of daily profiles for T1D-MDI, T1D-PMP, T1D-CLC, T2D-MDI, and Healthy subgroups; pairwise comparisons (with Bonferroni correction) between T1D-MDI, T1D-PMP, T1D-CLC, T2D-MDI, and Healthy subgroups;

FIG. 10 shows exemplary two steps in the iterative process to determine the “optimal” set of CSCs, wherein each step uses a different data set (the Training data set to identify a candidate set of CSCs and the Validation data set to evaluate the candidate set of CSCs);

FIG. 11 shows exemplary visualization of all 35 CSC centroids ordered by TIR, with the centroid with the highest TIR on the left and the centroid with the lowest TIR on the right;

FIG. 12 shows exemplary scatterplots of the points for which result from using to classify the 141,867 daily CGM profiles of the Testing data set;

FIG. 13 shows exemplary Hexbin plots of the pairs of points (ūᵢ, m̅ᵢ), wherein the plots for ‘All Individuals’, ‘Healthy Individuals’, and ‘T1D-CSII Individuals’ use a log-scale for the color scale;

FIG. 14 is an exemplary 3-panel plot which illustrates the progression of three individuals with T1D over 14 days;

FIG. 15 is an exemplary 4-panel plot that illustrates the ability of the set of CSCs to distinguish between states of health and treatment modalities;

FIGS. 16A, 16B, 16C, 16D, 16E, 16F, 16G, 16H, 16I, and 16J are exemplary Illustrations of relationships between CSC and AGP;

FIG. 17 shows an exemplary high-level functional block diagram for embodiments of the system;

FIG. 18 shows an exemplary network system in which embodiments of the system and method can be implemented;

FIG. 19 shows an exemplary a block diagram that illustrates a system including a computer system and the associated Internet connection upon which an embodiment may be implemented;

FIG. 20 shows an exemplary system in which one or more embodiments of the system and methods can be implemented using a network, or portions of a network, or computers; and

FIG. 21 shows an exemplary block diagram illustrating an example of a machine upon which one or more aspects of embodiments of the system and method can be implemented.

### DETAILED DESCRIPTION

Embodiments can relate to a system 100 for processing glucose data by efficient glucose database management. The system 100 can include a physical data store 102 containing glucose measurement data and a representation for at least one cluster of the glucose measurement data. The representation can approximate a glycemic profile vector array for a cluster of multiple glucose profiles segmented by plural time ranges. The system 100 can include a processor 104 and computer memory 106 configured with instructions 108 stored thereon that when executed will cause the processor 104 to implement any of the method steps disclosed herein. The instructions can cause the processor 104 to receive glucose measurements. The instructions can cause the processor 104 to convert the glucose measurements into vectorial form. The instructions can cause the processor 104 to search the physical data store 102 by comparing a newly received glucose measurement to a centroid of a cluster using a similarity metric. The instructions can cause the processor 104 to classify the newly received glucose measurement with a cluster having a matched similarity metric based on the comparing. The instructions can cause the processor 104 to ascribe a treatment to the newly received glucose measurement. The treatment can be a command signal, a modification signal, a recommendation, etc. for an insulin dose, a bolus dose, an exercise routine, a meal consumption routine, a medication routine, etc.

Instructions can cause the processor 104 to store the classification of the newly received glucose measurement in a data store 102 that is in communication with an other device(s) 110 (e.g., one or more of a predictive modeling system, a decision support system, an insulin delivery system, an insulin monitoring system, an automated control system, etc.) configured to use the classification as input. In addition, or in the alternative, instructions can cause the processor 104 to transmit the classification of the newly received glucose measurement to an other device(s) 110 (e.g., one or more of a predictive modeling system, a decision support system, an insulin delivery system, an insulin monitoring system, an automated control system, etc.) configured to use the classification as input. In addition, or in the alternative, instructions can cause the processor 104 to monitor, analyze, or influence a concentration of glucose levels in a fluid using the classification of the newly received glucose measurement.

In some embodiments, instructions can cause the processor 104 to receive the glucose measurements from a glucose measurement device or data source 112 (e.g., a glucose monitor/sensor, a continuous glucose monitor/sensor, an assay device, etc.).

In some embodiments, system 100 can include the glucose measurement device or data source 112.

In some embodiments, the system 100 can include the data store 102 that is in communication with the other device(s) 110 (e.g., one or more of the predictive modeling system, the decision support system, the insulin delivery system, the insulin monitoring system, the automated control system, etc.). In some embodiments, the system 100 can include the other device(s) 110 (e.g., one or more of a predictive modeling system, the decision support system, the insulin delivery system, the insulin monitoring system, the automated control system, etc.).

In some embodiments, instructions can cause the processor 104 to calculate a Euclidean distance between one or more newly received glucose measurement and one or more centroids as the similarity metric.

In some embodiments, the physical data store 102 can include plural clusters. The clusters can be generated by generating an array of glucose measurements for each time range. A plurality of arrays can form a glycemic profile vector. A weight can be assigned to an array. An iterative hierarchical clustering technique can be applied until one or more cluster is generated that approximates one or more glycemic profile vectors. Any one or more cluster of the plural clusters can be defined by a cluster's centroid.

In some embodiments, the iterative hierarchical clustering technique can compute an R² value by linear regression for an array and vary a weight to maximize the R² value.

In some embodiments, the plural time ranges can include five time ranges. For instance, the plural time ranges can be 1) Level 2 hypoglycemia below glucose measurement-1; 2) Level 1 hypoglycemia within a range from glucose measurement-2 and glucose measurement-3; 3) Target Range (TIR) within a range from glucose measurement-4 and glucose measurement-5; 4) Level 1 hyperglycemia within a range from glucose measurement-6 and glucose measurement-7; and 5) Level 2 hyperglycemia above glucose measurement-8. In a non-limiting example glucose measurement-1 can be 54 mg/dl; glucose measurement-2 can be 54 mg/dl; glucose measurement-3 can be 70 mg/dL; glucose measurement-4 can be 70 mg/dL; glucose measurement-5 can be 180 mg/dL; glucose measurement-6 can be 180 mg/dL; glucose measurement-7 can be 250 mg/dL; and glucose measurement-8 can be 250 mg/dL.

In some embodiments, the glucose measurements can include plural glucose profiles for an individual. Each glucose profile can include plural glucose measurements obtained for a predetermined time period. Instructions can cause the processor 104 to compile the plural glucose profiles into a single glucose measurement time series for an individual. Instructions can cause the processor 104 to classify one or more glucose profile using one or more cluster to generate a sequence of indices representing a classification of one or more glucose profile in the single glucose measurement time series.

In some embodiments, instructions cause the processor 104 to generate, using the sequence of indices, a trace representing glucose variability of the individual.

In some embodiments, instructions can cause the processor 104 to generate an approximated Ambulatory Glucose Report (AGP) using the sequence of indices.

In some embodiments, one or more glucose profile of the multiple glucose profiles can be a continuous monitoring glucose (CGM) profile including glucose measurements obtained over a 24-hour time period. In addition, one or more glucose profile of the plural glucose profiles for the individual can be a continuous monitoring glucose (CGM) profile including glucose measurements obtained over a 24-hour time period.

Embodiments can relate to a method for processing glucose data for efficient glucose database management. The method can involve receiving glucose measurements. The method can involve converting the glucose measurements into vectorial form. The method can involve searching a physical data store 102 by comparing a newly received glucose measurement to a centroid of a cluster using a similarity metric. The physical data store 102 can contain glucose measurement data and a representation for at least one cluster of the glucose measurement data. The representation can approximate a glycemic profile vector for a cluster of multiple glucose profiles segmented by plural time ranges. The method can involve classifying the newly received glucose measurement with a cluster having a matched similarity metric based on the comparing. The method can involve ascribing a treatment to the newly received glucose measurement. The treatment can be a command signal, a modification signal, a recommendation, etc. for an insulin dose, a bolus dose, an exercise routine, a meal consumption routine, a medication routine, etc.

In some embodiments, the method can involve calculating a Euclidean distance between one or more newly received glucose measurement and one or more centroid as the similarity metric.

In some embodiments, the physical data store 102 can contain plural clusters generated by: 1) generating an array of glucose measurements for each time range, a plurality of arrays forming a glycemic profile vector; 2) assigning a weight to an array; and 3) applying an iterative hierarchical clustering technique that varies a weight until one or more cluster is generated that approximates one or more glycemic profile vector; 4) defining a cluster of the cluster set by the cluster's centroid.

The method can involve computing, via the iterative hierarchical clustering technique, an R² value by linear regression for an array and varying a weight to maximize the R² value.

As can be appreciated from the present disclosure, embodiments relate to a system 100 and method for processing glucose data by efficient database management. This can be done to classify glucose data and use the classified glucose data to monitor, analyze, influence, etc. a concentration of glucose level in a fluid. Some embodiments can relate to methods and systems for developing a database for classification and some embodiments can relate to methods and systems for implementing processes using the database.

Embodiments of the system 100 include a processor 104 configured to build a database of clustered data for glucose measurement classification and/or implement processes to classify glucose measurements. The processor 104 can be any of the processors 104 disclosed herein. The processor 104 can be part of or in communication with a machine 2000 (logic, one or more components, circuits (e.g., modules), or mechanisms). The processor 104 can be hardware (e.g., processor, integrated circuit, central processing unit, microprocessor, core processor, computer device, etc.), firmware, software, etc. configured to perform operations by execution of instructions embodied in algorithms, data processing program logic, artificial intelligence programming, automated reasoning programming, etc. It should be noted that use of processors 104 herein includes any one or combination of a Graphics Processing Unit (GPU), a Field Programmable Gate Array (FPGA), a Central Processing Unit (CPU), etc. The processor 104 can include one or more processing modules. A processing module can be a software or firmware operating module configured to implement any of the method steps disclosed herein. The processing module can be embodied as software and stored in memory, the memory being operatively associated with the processor 104. A processing module can be embodied as a web application, a desktop application, a console application, etc. Exemplary embodiments of the processor 104 and the machine 2000 are discussed later.

The processor 104 can include or be associated with a computer or machine readable medium 2002. As discussed in more detail later, the computer or machine readable medium 2002 can include memory 106. Any of the memory 106 discussed herein can be computer readable memory configured to store data. The memory 106 can include a volatile or non-volatile, transitory or non-transitory memory, and be embodied as an in-memory, an active memory, a cloud memory, etc. Embodiments of the memory 106 can include a processor module and other circuitry to allow for the transfer of data to and from the memory 106, which can include to and from other components of a communication system. This transfer can be via hardwire or wireless transmission. The communication system can include transceivers, which can be used in combination with switches, receivers, transmitters, routers, gateways, wave-guides, etc. to facilitate communications via a communication approach or protocol for controlled and coordinated signal transmission and processing to any other component or combination of components of the communication system. The transmission can be via a communication link. The communication link can be electronic-based, optical-based, opto-electronic-based, quantum-based, etc.

The computer or machine readable medium 2002 can be configured to store one or more instructions 108 thereon. The instructions 108 can be in the form of algorithms, program logic, etc. that cause the processor 104 to build and/or implement a classification model.

The processor 104 can be in communication with other processor(s) of other device(s) 110 (e.g., a predictive modeling system, a decision support system, an insulin delivery system, an insulin recommendation system, a glycemic state or insulin monitoring system, a glucose or insulin management system, an automated control system, etc.) configured to use the classification as input. Any of those other device(s) 110 can include any of the exemplary processors disclosed herein. Any of the processors can have transceivers or other communication devices/circuitry to facilitate transmission and reception of wireless signals. Any of the processors can include an Application Programming Interface (API) as a software intermediary that allows two applications to talk to each other. Use of an API can allow software of the processor 104 of the system 100 to communicate with software of the processor of the other device(s) 110.

Any of the transmissions between processors/devices/systems/modules can be a push, operation, a pull operation, or a combination of both. Any of the transmissions can be direct transmission between two components or transmission via an intermediary. An intermediary may be memory, database, data store, etc. for example. For instance, data from one processor may be transmitted to a database for storage before being transmitted to another processor.

As another example, data may be transmitted to an intermediary processor or processing module to process the data, format the data, encode the data, etc. before being transmitted to another processor. Data transmission between components can be continuously, periodically, at some other predetermined schedule, as-demanded by control signals, based on a condition being met per algorithmic function, etc.

### Exemplary Systems and Methods for Developing a Database of Clustered Data Sets

Embodiment can relate to a system 100 for developing a database to classify glucose data. The system 100 can include a processor 104. The system 100 can include computer memory 106 having instructions 108 stored thereon that when executed will cause the processor 104 to implement any of the method steps disclosed herein. The instructions 108 can cause the processor 104 to receive glucose profile data. The glucose profile data can include one or more glucose measurements. The glucose measurements can be a time series of measurements that represent a glucose level profile (e.g., a pattern, a behavior, a trend, etc.).

The glucose profile data can be historical, current, and/or real-time data. The glucose profile data is received by the processor 104. This can be done continuously, periodically, or at some other predetermined schedule. The glucose profile data can be pulled by the processor 104 from a data source 112 and/or pushed from the data source 112 to the processor 104. The data source 112 can be a device that generates glucose measurements (e.g., a glucose monitor/sensor, a continuous glucose monitor/sensor, an assay device, etc.) or a data store 102 (e.g., database) that stores glucose profile data. The glucose measurements can be of a fluid, such as interstitial fluid, etc. The processor 104 can store the glucose profile data in transient or persistent memory for later processing or process the glucose profile data as it is being received. For instance, the processor 104 can receive glucose profile data and aggregate the glucose profile data in storage. The aggregation can be based on the type of data, what the data represents, the time of receiving the data, the time the data was generated, etc., which can be embodied in metadata for example.

The instructions 108 can cause the processor 104 to generate a set of clusters from glucose profile data. As a non-limiting example, the instructions can cause the processor 104 to perform a machine learning data mining technique that divides groups of objects in the glucose profile data into classes of similar objects. The clusters can be configured to approximate plural time in ranges of glucose profile data. For instance, the clustering can be done so that one or more of the clusters approximate one or more time in ranges in the glucose profile data. A time in range can be a time duration in which a glucose measurement of glucose profile data has a value within a range of glucose measurements. For instance, there may be a time duration in which the glucose profile data has a glucose measurement of G1 and G1 falls within glucose measurement range x-y. There can be plural time in ranges set. In other words, it can be beneficial to know when and how much of the glucose profile data had glucose measurements that fall within predetermined time in ranges. This information can be used to generate clusters representing the same.

The instructions 108 can cause the processor 104 to generate one or more sets of clusters. Any of the sets of clusters can be generated using a hierarchical clustering technique.

For instance, a set of clusters can be generated by generating an array of glucose measurements for each time in range. One or more arrays can form a vector. For instance, it is contemplated for there to be five time in ranges, which will generate five arrays. More or less time in ranges (and arrays) can be used. One or more arrays (e.g., all five arrays) can be used to generate a vector. Because the arrays comprise glucose measurements, the vector can be a glycemic profile vector (or one or more glycemic profile vectors). A weight can be assigned to an array, which can include assigning a weight to one or more arrays. The weight can be a value from 0 to 1 for example, a weight function, or any other mathematical operator that gives an array a desired influence or effect. Any one or combination of weight can be determined by an optimization function, objective function, cost function, etc. Any one or combination of weights can be fixed or variable. The weights can be variable and randomly set to an arbitrary value for the first or initial iteration. The weights can be varied at each iteration until an optimal is reached. For instance, the instructions 108 can cause the processor 104 to apply an iterative hierarchical clustering technique that varies weights until a cluster set is generated that approximates the glycemic profile vector(s). This approximation can be the best approximation, a desired approximation, an optimal approximation, etc. For instance, an optimal approximation can be one defined by an optimizing function, an objective function, a cost function, etc.

The instructions 108 can cause the processor 104 to define a cluster (which can include any number of clusters) of the cluster set by the cluster's centroid. For instance, an individual cluster of the cluster set can be defined by an individual centroid of that individual cluster. This can be done for one or more of the clusters. The centroid can be a statistical (weighted or unweighted) middle, mean, mode, etc. Thus, each cluster can be defined by a value or variable representative of the cluster's centroid. The cluster set can be a set of values or variables representative of the clusters. As noted herein, each value or variable is representative of the time in range, or is an approximation of the time in range for the glucose profile data. Notably, the cluster data is an accurate representation (or proxy) for the time in ranges of the glucose data profile but with a significantly reduced data set.

As can be appreciated, the system 100 can reduce data needed for glucose analyses, reduce computational resources required for systems processing such data, etc. For instance, instead of transmitting/processing a daily continuous glucose monitoring (CGM) profile (which is typically 288 data points), a single number can be transmitted/processed. The system 100 can generate cluster data from any type of glucose measurement system (e.g., data from any type of measurement system, data from disparate glucose measurement systems, data that is not normalized, etc.) and data pertaining to one or more of type 1 diabetes, type 2 diabetes, etc. In other words, the system 100 can be agnostic to the types of data, the modes of measurement, etc. The system 100 improves robustness and accuracy in that glucose profile data that would otherwise be considered inadequate due to missing data, data being from disparate data sources, or data not being normalized, etc. can be used to generate the clusters.

The instructions 108 can cause the processor 104 to store the cluster set in a data store 102. This can be a physical data store 102. The data store 102 can be in communication with an other device(s) 110 (e.g., one or more of a predictive modeling system, a decision support system, an insulin delivery system, an insulin monitoring system, an automated control system, etc.) configured to use one or more clusters of the cluster set as input. For instance, and as will be explained in more detail later, a decision support system can compare new glucose profile data to the model to classify the new glucose profile data as falling within one or more of the time in ranges and doing so by assigning one or more centroid values to the new glucose profile data—e.g., the new glucose profile data can be matched with one or more clusters and given a centroid cluster value(s) with which it is matched. Alternatively, the processor 104 can perform this function and transmit the value(s) to the decision support system. This value(s) is/are then used as a proxy(ies) or surrogate(s) for the time in range(s) for the glucose measurements of the glucose profile. The data store 102 can be part of the system 100 or part of another system. In addition, or in the alternative, the system 100 can be a predictive modeling system, a decision support system, an insulin delivery system, an insulin monitoring system, an automated control system, etc., and use the data directly (e.g., obviate the use of a data store 102). The cluster set can be stored or used as the classification database. This database can be modified, learned, etc. based on additional or updated data.

The glucose profile data can include plural glucose profiles. The system 100 can classify one or more glucose profile of the plural glucose profiles by one or more clusters of the cluster set. One or more of the glucose profiles can include plural glucose measurements obtained for a predetermined time period. For instance, one or more glucose profile can be a continuous monitoring glucose (CGM) profile. One or more of the CGM profile can including glucose measurements obtained over a 24-hour time period, which can include glucose measurements taken every 5 minutes over a 24-hour time period. As noted herein, the system 100 can operate with missing data. Thus, while it may be anticipated that each CGM profile data comprises 288 glucose measurements (e., 288 data points), the system 100 can generate useful clusters with less.

The instructions 108 can cause the processor 104 to computer an R² value by linear regression for each array when implementing the iterative hierarchical clustering technique. The instructions 108 can cause the processor 104 to vary one or more weight to maximize the R² value. The varying of weight(s) can be via an iterative or recursive process, which may be governed by an optimization function, an objective function, a cost function, etc.

While other numbers of time in ranges and ranges for the time in ranges can be used, it is contemplated for the plural time in ranges to includes five time in ranges. These can be:
Level 2 hypoglycemia below glucose measurement-1;
Level 1 hypoglycemia within a range from glucose measurement-2 and glucose measurement-3;
Target Range (TIR) within a range from glucose measurement-4 and glucose measurement-5;
Level 1 hyperglycemia within a range from glucose measurement-6 and glucose measurement-7; and
Level 2 hyperglycemia above glucose measurement-8.

The time in ranges can be:
glucose measurement-1 is 54 mg/dl;
glucose measurement-2 is 54 mg/dl;
glucose measurement-3 is 70 mg/dL;
glucose measurement-4 is 70 mg/dL;
glucose measurement-5 is 180 mg/dL;
glucose measurement-6 is 180 mg/dL;
glucose measurement-7 is 250 mg/dL; and
glucose measurement-8 is 250 mg/dL.

In developing the database, the glucose profile data can include glucose measurements from one or more individual. There can be one or more glucose profile for each individual. This robust data set can allow the model to be used to determine glycemic trends, predict glycemic states, use multivariate analyses regarding conditions and factors (e.g., eating behavior, exercise behavior, medical condition, age, gender, race, heart rate, respiratory rate, blood oxygen saturation, etc.) that cause or relate to a glycemic state, etc. For instance, multivariable modeling techniques can be used to determine which conditions or factors statistically contribute to a change glycemic state, a change in risk of hypo- or hyper-glycemia, etc., which can also be used to estimate the probabilities of the same. The multivariable modeling technique can include one or more of logistic regression with or without cubic splines, random forest, xgboost, support vector machines, nearest neighbor, artificial neural networks, and/or long short-term memory (LSTM), multivariate analysis of variance (MANOVA), multivariate analysis of covariance (MANCOVA), principal components analysis (PCA), canonical correlation analysis, redundancy analysis (RDA), correspondence analysis (CA), canonical correspondence analysis (CCA), multidimensional scaling, discriminant analysis, linear discriminant analysis (LDA), clustering systems, recursive adaptive partitioning, vector autoregression, principal response curves analysis (PRC), etc. As a non-limiting example, the means, standard deviations, and/or cross correlations one or more of the conditions or factors and the cluster centroids can be fit with a logistic ridge regression model using cubic splines, for example, to generate an output that is an estimation or probability that a glycemic state will occur.

As noted above, there can be one or more glucose profile for each individual. If there are plural glucose profiles for an individual, the instructions 108 can cause the processor 104 to compile plural glucose profiles into a single glucose measurement time series for an individual—e.g., a single time series of glucose measurements spanning the entire set of glucose profiles for that individual. The instructions 108 can cause the processor 104 to classify each glucose profile by one or more cluster of the cluster set to generate a sequence of indices representing a classification of each glucose profile in the single glucose measurement time series. The instructions 108 can cause the processor 104 to store the sequence of indices in the data store 102 to be part of the database. This can be done for one or more individual. Thus, the data store 102 can have a sequence of indices for each individual, each sequence being an approximation of the time in ranges of the glucose measurements in their respective time series. It should be noted that there can be one or more time series of data for an individual. Also, there can be one or more sequence of indices for any single time series of data.

The sequence of indices can allow the database to be used to determine glycemic trends, predict glycemic states, use multivariate analyses regarding conditions and factors (e.g., eating behavior, exercise behavior, medical condition, age, gender, race, heart rate, respiratory rate, blood oxygen saturation, etc.) that cause or relate to a glycemic state, etc. for an individual. In addition, or in the alternative, the instructions 108 can cause the processor 104 generate, using the sequence of indices, a trace representing glucose variability of the individual. This can be done for one or more individual. Also, there can be one or more trace for an individual. The instructions 108 can cause the processor 104 to store the trace in the data store 102 to be part of the database.

### Exemplary Systems and Methods for Classifying Glucose Data

Embodiments can relate to a system 100 for classifying glucose data. The system 100 can be configured to implement an embodiment of the methods disclosed herein using the database of clustered data to classify glucose data. The system 100 can include a processor 104. The system 100 can include computer memory 106 having instructions 108 stored thereon that when executed will cause the processor 104 to implement or apply an embodiment of the methods disclosed herein. The instructions 108 can cause the processor 104 to receive glucose profile data including plural glucose measurements. It is contemplated for the glucose data to be of a single individual so as to assess or evaluate a glycemic state of that individual by comparing the individual's glucose profile data to clusters in the database; however, the glucose profile data can be of one or more individual. It is contemplated for the glucose profile data to be recent data (e.g., data collected in real-time or within the past 24 hours) but the glucose profile data can be historical, current, and/or real-time data.

The instructions 108 can cause the processor 104 to classify the glucose profile data, or a portion thereof, by comparing glucose profile data to an embodiment of the database. The database can include a set of clusters configured to approximate one or more glycemic profile vectors for the individual and/or for a group of individuals the individual falls under (e.g., the individual may be grouped by age, gender, race, medical condition, etc.). The glycemic profile vector(s) are arrays of previously processed glucose profile data segmented by plural time in ranges. The previously processed glucose profile data includes historical glucose data, but can also include current or real-time glucose data. The previously processed glucose data can be data of the individual, data of individuals within the individual's groups (which may or may not include the individual's data), data of individuals that may or may not be within the individual's group, etc. One or more of the time in ranges can be a time duration in which a glucose measurement of previously processed glucose profile data had a value within a range of glucose measurements.

The instructions 108 can cause the processor 104 to store the classification of glucose profile data in a data store 102 that is in communication with an other device(s) 110 (e.g., one or more of a predictive modeling system, a decision support system, an insulin delivery system, an insulin monitoring system, an automated control system, etc.) configured to use the classification as input. In addition, or in the alternative, the instructions 108 can cause the processor 104 to transmit the classification of glucose profile data to an other device(s) 110 (e.g., one or more of a decision support system, an insulin delivery system, an insulin monitoring system, etc.) configured to use the classification as input. In addition, or in the alternative, the instructions 108 can cause the processor 104 to monitor, analyze, and/or influence a concentration of glucose levels in a fluid using the classification.

The instructions 108 can cause the processor 104 to classify glucose profile data by comparing glucose profile data to centroids of clusters of the set of clusters. For instance, glucose profile data that exactly, approximately, or similarly matches with a centroid can be classified as having an exact, approximate, or similar time in range pattern as the cluster to which the centroid belongs. The glucose profile data for any one individual can have one or more classifications. The glucose profile data for any one individual can comprise one or more glucose profile for the individual. There can be one or more classification for any one glucose profile. It is contemplated for the glucose profile that is being classified to have only one classification (i.e., it matches the centroid of one cluster the best). In an unlikely event of a tied similarity score, the first match can be selected.

The model can have a set of clusters. The number of clusters can be 35, for example. More or less clusters per set can be used. There can be one or more sets of clusters. The number of clusters in one set can be the same as or different from the number of clusters in another set. The number of clusters, the number of sets, etc. can be set by desired design criteria (e.g., optimization, computational resources, processing speed, accuracy, robustness, etc.). The comparison can be comparing the glucose profile data to one or more clusters (or centroids) within the same set, within different sets, clusters of a single set, clusters of multiple sets, etc.

The instructions 108 can cause the processor 104 to compare glucose profile data to one or more centroid using a similarity metric. The cluster(s) having the best similarity metric can be used to classify glucose profile data. The similarity metric can be a numerical value falling within a range of value (e.g., from 0-1). A similarity metric of 0 can indicate a match, whereas a similarity metric of 1 can indicate a mismatch with a gradation of degree of matching between 0 and 1. Alternatively, a similarity metric of 1 can indicate a match, whereas a similarity metric of 0 can indicate a mismatch with a gradation of degree of matching between 1 and 0. Other similarity metric schemes can be used. In an exemplary embodiment, the instructions 108 can cause the processor 104 to calculate a Euclidean distance between one or more glucose profile data points and one or more centroids as a similarity metric. The distance(s) can be normalized to fit within the 0 to 1 range, for example.

The glucose profile data can include one or more glucose profiles for an individual. Each glucose profile can include plural glucose measurements obtained for a predetermined time period (e.g., over a 24 hour time period). The instructions 108 can cause the processor 104 to compile the plural glucose profiles into a single glucose measurement time series for the individual. The instructions 108 can cause the processor 104 to classify each glucose profile by one or more cluster of the cluster set(s) to generate a sequence of indices representing the classification of each glucose profile in a single glucose measurement time series. The instructions 108 can cause the processor 104 to store the sequence of indices in a data store 102 that is in communication with an other device(s) 110 (e.g., one or more of a predictive modeling system, a decision support system, an insulin delivery system, an insulin monitoring system, an automated control system, etc.) configured to use the classification as input. In addition, or in the alternative, the instructions 108 can cause the processor 104 to transmit the sequence of indices to an other device(s) 110 (e.g., one or more of a predictive modeling system, a decision support system, an insulin delivery system, an insulin monitoring system, an automated control system, etc.) configured to use the classification as input. In addition, or in the alternative, the instructions 108 can cause the processor 104 to monitor, analyze, and/or influence a concentration of glucose levels in a fluid using the classification.

The instructions 108 can cause the processor 104 to generate an approximated Ambulatory Glucose Report (AGP) using the sequence of indices. In addition, or in the alternative, an other device(s) 110 (e.g., one or more of a predictive modeling system, a decision support system, an insulin delivery system, an insulin monitoring system, an automated control system, etc.) can generate an approximated Ambulatory Glucose Report (AGP) using the sequence of indices.

As noted herein, the glucose profile data can include plural glucose profiles, each glucose profile including plural glucose measurements obtained for a predetermined time period. For instance, each glucose profile can be a continuous monitoring glucose (CGM) profile including glucose measurements obtained over a 24-hour time period.

While other numbers of time in ranges and ranges for the time in ranges can be used, it is contemplated for the plural time in ranges to includes five time in ranges. These can be:
Level 2 hypoglycemia below glucose measurement-1;
Level 1 hypoglycemia within a range from glucose measurement-2 and glucose measurement-3;
Target Range (TIR) within a range from glucose measurement-4 and glucose measurement-5;
Level 1 hyperglycemia within a range from glucose measurement-6 and glucose measurement-7; and
Level 2 hyperglycemia above glucose measurement-8.

The time in ranges can be:
glucose measurement-1 is 54 mg/dl;
glucose measurement-2 is 54 mg/dl;
glucose measurement-3 is 70 mg/dL;
glucose measurement-4 is 70 mg/dL;
glucose measurement-5 is 180 mg/dL;
glucose measurement-6 is 180 mg/dL;
glucose measurement-7 is 250 mg/dL; and
glucose measurement-8 is 250 mg/dL.

Any of the other device(s) 110 can be configured to generate an output based on the classification input. In some embodiments, the other device(s) 110 can be part of the system 100—e.g., the system 100 can include the other device(s) 110. The classification can be used by the processor 104 or a processor of the other device(s) 110 to generate a signal:
a. Recommending or implementing a process to obtain additional data (e.g., a signal is generated requiring additional patient data, insulin delivery data, metabolic data, etc.);
b. Recommending or implementing a process to initiate preventative or mitigating measures (e.g., a signal is generated to modify insulin rate, modify behavior, etc.);
c. Recommending or implementing a process to initiate enhanced monitoring (e.g., a signal is generated to inform a user that the risk of hypoglycemia is heightened and additional monitoring should occur).
d. That is an alert signal or a command signal to an insulin delivery device to modify insulin rate or dosage, etc.

The system 100 or any of the other device(s) 110 can include a display configured to generate a user interface. A user can control aspects of the system 100 via the user interface.

In addition, the user interface can display aspects of the classification and other outputs, generate graphical displays, audible, graphical or textual alerts, etc.

The system 100 can include the processor 104 in combination with one or more data stores 102. The data store 102 can be configured to contain plural classification databases. For instance, the system 100 can be configured to generate plural classification databases. The processor 104 can be configured to use any one or combination of the plural classification databases. Each classification database can be generated based on the glucose and other patient data available, the anticipated availability of glucose or other patient data, the quality (how reliable the data is) of glucose or other patient data, the frequency (how often it is generated or available) of glucose or other patient data, dimensionality (how many attributes or variables the data has) of the glucose or other patient data, etc. For instance, a first classification database can be generated for a data set in which certain type of data is sparse but other type of data is abundant, a second classification database can be generated for a data set in which the reliability of certain data is low but is high for other type of data, etc. The type of patient data can include from which data source 112 the data is received or attempted (or desired) to be received, which attributes are included in the data, the number of attributes the data has, etc. A classification database can be generated for anticipated data flows, thereby generating plural classification databases. The plural classification databases can be stored in one or more data store 102. The processor 104 can be in communication with the data store(s) 102 to as to access any one or combination of the plural classification databases.

The processor 104 can be configured to switch from a first classification database to a second classification database for implementation based on at least one or more of: a type of data, the availability of data, reliability of data, etc. The processor 104 can detect the change (e.g., based on the metadata) and switch classification databases.

The processor 104 can be configured to update the classification database based on new data. As noted above, the glucose profile data can be historical, current, and/or real-time data, and can be received continuously, periodically, or at some other predetermined schedule and can include information about glycemic episodes, treatment, etc. The system 100 can update any one or combination of the classification databases based on updated data. The updated classification database can replace the already existing classification database in the data store 102. Alternatively, if the updated classification database is sufficiently different or is better suited for a patient data scenario than any other existing classification database, the updated classification database can be added amongst the plural classification databases.

As can be appreciated from the present disclosure, an aspect of an embodiment of the present invention provides, among other things, a system, method and computer readable medium for identifying clinically-similar clusters of daily continuous glucose monitoring (CGM) profiles.

An aspect of an embodiment of the present invention provides, among other things, a system, method and computer readable medium for performing the following: a) constructing and then fixing, a set of Clinically-Similar Clusters (CSCs), with the property that for any other daily continuous glucose monitoring (CGM) profile there is a Clinically-Similar Cluster (CSC) that approximates the time in ranges of said daily CGM profile, and b) determining an approximation of any daily CGM profile by a CSC.

An aspect of an embodiment of the present invention system, method, and computer readable medium comprises, for example but not limited thereto, two steps. For example, a first step may include: constructing and then fixing, a set of Clinically-Similar Clusters (CSCs), with the property that for any other daily CGM profile there is a Clinically-Similar Cluster (CSC) that approximates the time in ranges of said daily CGM profile, thereby preserving key clinically-relevant characteristics of the daily CGM profile.

The set may be defined using hierarchical clustering, where weighting of the input columns is varied until the set of CSCs have the desired performance when approximating the time in ranges of daily CGM profiles. A second step, for example, may include determining an approximation of any daily continuous glucose monitoring (CGM) profile by a CSC, which may involve computing a similarity metric (e.g., Euclidean distance) between the candidate daily CGM profile and the centroids of each CSC, and selecting the single CSC with the minimal similarity metric value.

In an embodiment, when these steps are accomplished, any daily CGM profile can be mapped to a CSC, and the sequence of CSCs for an individual can then be used as a surrogate for the Ambulatory Glucose Profile (AGP) of this individual, and the associated time in ranges of the original daily CGM profile. In addition to AGP and its associated metrics, the sequence of CSCs provides information about the timing and the inter-day variability of the clinically-relevant glycemic events of a patient. Potential applications of an aspect of an embodiment of the present invention system, method, and computer readable medium include, but not limited thereto, one or more of the following: (i) Data structuring and dimensionality reduction; (ii) Database indexing; (iii) Compression/encryption of daily CGM profiles; (iv) Distinguishing between health states and treatment modalities; (v) CGM replacement for common clinical tests; (vi) CGM pattern recognition and forecast; or (vii) Tracking disease progression.

An aspect of an embodiment of the present invention system, method, and computer readable medium may be configured to, among other things, work with daily CGM profiles generated by sensors with different sampling resolutions, and to daily CGM profiles which have missing data, up to a certain threshold. One of the significant advantages of an aspect of an embodiment of the present invention is, but not limited thereto, the ability to classify all CGM daily profiles into a relatively small, finite, and fixed across patient groups and health state, set of CSCs describing well the clinical status of these patients. An aspect of an embodiment of the present invention also adds, but not limited thereto, a time-variation component to commonly accepted CGM data representations, such as the AGP and its associated time in ranges.

An aspect of an embodiment of the present invention provides a system, method, and computer readable medium for, among other things, identifying clinically-similar clusters of daily CGM Profiles.

An aspect of an embodiment of the present invention provides a system, method, and computer readable medium for, among other things, providing the classification of daily CGM profiles and its clinical interpretation.

An aspect of an embodiment of the present invention provides a system, method, and computer readable medium for, among other things, defining a set of CSCs where any daily CGM profile can be classified as one of the CSCs and where the CSCs reliably approximate the clinical characteristics of the daily CGM profiles.

An aspect of an embodiment of the present invention provides a system, method, and computer readable medium for, among other things, providing CSCs that clearly distinguish between health states and treatment modalities, and can be used as a representation of glycemic volatility of a person over time.

An aspect relates to a method for identifying clinically-similar clusters of daily continuous glucose monitoring (CGM) profiles, as described herein. The method can involve:
obtaining an individual i wherein each individual i has a single CGM time series generated during the study they take part in;
classifying all of the daily CGM profiles in the individual's single time series that results in a sequence sᵢ of (possibly non-consecutive) indices indicating the CSC that each daily CGM profile is classified as;
wherein each entry of the sequence sᵢ corresponds to a single day of observation and the days are ordered by date of occurrence and wherein fᵢ(t) be the number of unique CSCs visited by individual i after t days of observation;
wherein fᵢ is the trace of the number of unique CSCs visited by individual i over time and provides an idea of the variability in the individual's blood glucose;
providing a mean trace that indicates the average behavior of individuals in a subgroup and can help highlight the differences in behavior between different subgroups; wherein for a given subgroup G, the mean value is

$$\overline{{f}_{G}} = \frac{1}{\vert I \vert} {\sum }_{i = 1}^{\vert I \vert} {f}_{i} ( t )$$

where |I| is the total number of individuals in the subgroup; wherein not all individuals in a subgroup have the same number of days observed in their sequences sᵢ of CSC indices; wherein as such, f̅_̅{̅G̅}̅ is defined only if there is a minimum number of sequences at time t; and wherein the minimum number of sequences is a function of the subgroup G; and
distinguishing between both health state and treatment modality because they track glycemic volatility; wherein on average healthy individuals have the least glycemic volatility, followed by individuals with T2D and then by individuals with T1D; and wherein for individuals with T1D, individuals using MDI as the treatment modality on average have the largest glycemic volatility followed by individuals on PMP and then those individuals on CLC.

The method can further involve:
data structuring and dimensionality reducing wherein the multitude of all possible said daily CGM profiles, as clinically represented by AGP and their time in ranges, is reduced to a finite and fixed set of CSCs;
database indexing, wherein a database is indexed by the structure defined by said CSCs that will ensure fast and efficient search for subgroups of similar daily CGM profiles;
compressing and/or encrypting of said daily CGM profiles;
distinguishing between health states and treatment modalities; and
wherein the ability of said CSCs to distinguish with high fidelity between health states serves as a replacement of clinical tests for a specified number of days of CGM wear in home environment, accompanied by a predefined schedule of meals and physical activity, that will achieve diagnostic results.

An aspect relates to a method for performing the following: a) constructing and then fixing, a set of Clinically-Similar Clusters (CSCs), with the property that for any other daily continuous glucose monitoring (CGM) profile there is a Clinically-Similar Cluster (CSC) that approximates the time in ranges of said daily CGM profile, and b) determining an approximation of any daily CGM profile by a CSC, as described herein.

An aspect relates to a system for performing the following: a) constructing and then fixing, a set of Clinically-Similar Clusters (CSCs), with the property that for any other daily continuous glucose monitoring (CGM) profile there is a Clinically-Similar Cluster (CSC) that approximates the time in ranges of said daily CGM profile, and b) determining an approximation of any daily CGM profile by a CSC, as described herein.

An aspect relates to a computer-readable storage medium having computer-executable instructions stored thereon which, when executed by one or more processors, cause one or more computers to perform functions for performing the following: a) constructing and then fixing, a set of Clinically-Similar Clusters (CSCs), with the property that for any other daily continuous glucose monitoring (CGM) profile there is a Clinically-Similar Cluster (CSC) that approximates the time in ranges of said daily CGM profile, and b) determining an approximation of any daily CGM profile by a CSC, as described herein.

An aspect relates to a method configured to present a two-step iterative process to identify a fixed set of clinically-similar clusters (CSCs) of daily CGM profiles. The two-step process uses hierarchical clustering on a Training data set configured to identify candidate sets of CSCs. The two-step process uses a Validation data set configured to evaluate the performance of the candidate set of CSCs. The ability of the CSCs to faithfully capture the five different times in ranges of the daily CGM profiles being classified is evaluated. The fixed set of 35 CSCs, Ψ, is then used to classify the daily CGM profiles in a separate Testing data set, and wherein the results indicated that the set is robust and generalizes well.

In some embodiments, the distribution of daily CGM profiles to the different CSCs is shown to be specific to health state and treatment modality.

An aspect relates to a method of visualizing individual glycemic control. The clinically-similar clusters (CSCs) can be used to visualize differences in glycemic control between individuals who have the same health state and treatment modality, for identifying individuals who may need more personalized attention. In some embodiments,

$$= \frac{{u}_{i}}{k}$$

where uᵢ is the number of unique CSCs that are needed to classify k daily CGM profiles of individual i.

In some embodiments:
the total number of unique CSCs that may be bounded (i.e., there are just 35 different CSCs), if k is large, then ûᵢ will tend to 0;
k is fixed at 28 daily profiles (i.e., 4 weeks of data); and
ūᵢ is defined as the average of the values ûᵢ generated by computing each ûᵢ using a sliding window of k=28 daily CGM profiles, where the sliding window advances by 7 days (1 week of data), and where each sliding window must have at least 14 daily CGM profiles (2 weeks of data) for ûᵢ to be computed.

In some embodiments, mi is the average CSC index of k daily CGM profiles of individual i, and let m̅ᵢ be the average of the values mᵢ generated by computing each mi using a sliding window of k=28 daily CGM profiles, where the sliding window advances by 7 days and where each sliding window must have at least 14 daily CGM profiles for m̅ᵢ to be computed.

In some embodiments, any daily CGM profile can be approximated by one of 35 prefixed clinically-similar clusters (or specified number of prefixed clinically-similar clusters. Said approximation means that when the daily CGM profile is classified into a CSC, the CSC preserves the information carried by the original daily CGM profile, in terms of the time-in-range system of metrics. The CSCs expand, and to some extent complete, the interpretation of CGM data provided by the AGP/TIR system—wherein the AGP/TIR is a static snapshot of 14 days (or specified number of days) of data, the sequence of CSCs derived from the same data tracks the progression of glycemic control over time. The time series of CSCs over 14 days (or specified number of days) illustrate how stable or volatile the glycemic control of the person is.

An aspect of an embodiment of the present invention system, method, and computer readable medium generally relates to, but not limited thereto, medicine and medical devices, as used for insulin treatment of diabetes mellitus and other metabolic disorders, including but not limited to type 1 and type 2 diabetes, type 2 (T1D, T2D), latent autoimmune diabetes in adults (LADA), postprandial or reactive hyperglycemia, or insulin resistance. In alternative embodiments, an aspect of an embodiment of the invention defines, and then fixes, a set of Clinically-Similar Clusters (CSCs), with the property that for any other daily CGM profile there is a Clinically-Similar Cluster (CSC) that approximates the time in ranges of said daily CGM profile, thereby preserving key clinically-relevant characteristics of the daily CGM profile. When the CSCs are defined and fixed, any daily CGM profile can be mapped to a CSC, and the sequence of CSCs for an individual can then be used as a surrogate for the Ambulatory Glucose Profile (AGP) of this individual, and the associated time in ranges of the original daily CGM profile. In addition to AGP and its associated metrics, the sequence of CSCs provides information about the timing and the inter-day variability of the clinically-relevant glycemic events of a patient. One of the significant advantages of an aspect of an embodiment of the present invention is, but not limited thereto, the ability to classify all CGM daily profiles into a relatively small, finite, and fixed across patient groups and health state, set of CSCs describing well the clinical status of these patients.

### EXAMPLES

The following are examples of developing, testing, and implementing embodiments the systems and methods disclosed therein. The following are exemplary only and are not to be taken in a limiting sense.

### Example 1

### Data

The data used in this work came from:
1. The University of Virginia's Center for Diabetes Technology (namely the DCLP1 [19], DCLP3 [20], DIAMOND1 [21], DIAMOND2 [22], DSS1 [23], Nsc IGHTLIGHT [24], and TRIALNET studies), and
2. The Jaeb Center for Health Research website (the CITY [25], DCLP5 [26], NDIAB [27], MDEX [28], REPLACE-BG [29], RT-CGM [30], SENCE [31], SEVHYPO [32], and WISDM [33] studies).

From each of these studies, the procedure outlined in Section III. A. of [34] was used to process the CGM time series and define the daily CGM profiles, where a daily CGM profile is a time series of 288 blood glucose data points collected every 5 minutes during the midnight-to-midnight (24-hour) period. The 204,710 daily CGM profiles from these 16 different studies were used to form three different data sets, each with a distinct purpose:
1. The Training Data Set: This data set was composed of 23,916 daily CGM profiles taken from the DCLP1, DCLP3, DIAMOND1, DIAMOND2, DSS1, and NIGHTLIGHT studies and was used to define the candidate sets of CSCs.
2. The Validation data set: This data set was composed of 37,758 daily CGM profiles again taken from the DCLP1, DCLP3, DIAMOND1, DIAMOND2, DSS1, and NIGHTLIGHT studies and was used to a) assess the performance of each candidate set of CSCs, and b) select the final and fixed set of CSCs.
3. The Testing Data Set: This data set was composed of 143,036 daily CGM profiles taken from the CITY, DCLP5, DIAMOND2, NDIAB, MDEX, REPLACE-BG, RT-CGM, SENCE, SEVHYPO, TRIALNET and WISDM studies and was used to evaluate the robustness and generalizability of the final selected set of CSCs.

The studies represent healthy individuals, individuals with type 1 diabetes (T1D), and individuals with type 2 diabetes (T2D). The studies also represent a variety of treatment modalities including multiple daily injections (MDI), insulin pump (PMP), and closed loop control (CLC).

From the 16 data sets, there were 2,462 subjects and a total of 204,710 daily CGM profiles. The characteristics of the participants in each study are detailed in Table 1.

TABLE 1
Characteristics of the 16 data sets used in this work.

| | | No. Study | | | | | Study | No. Daily |
|---|---|---|---|---|---|---|---|---|
| | Health | Subjects | Age, | BMI, | HbA1c, | Treatment | Duration, | CGM |
| Study Name | State | (% Female) | years | kg/m² | % | Modalities | weeks | Profiles |
| CITY | T1D | 153 (49.7) | 17.5 (2.9) | 25.4 (5.0) | 9.1 (1.0) | MDI, PMP | 26 | 20,226 |
| DCLP1 | T1D | 125 (47.2) | 32.5 (14.9) | 26.7 (5.2) | 7.5 (0.9) | CLC, PMP | 13 | 5,459 |
| DCLP3 | T1D | 168 (50.0) | 32.8 (16.0) | 25.8 (5.3) | 7.6 (1.0) | CLC, PMP | 26 | 24,450 |
| DCLP5 | T1D | 93 (50.5) | 10.7 (2.0) | 19.4 (4.0) | 7.7 (1.1) | CLC, PMP | 16 | 10,050 |
| DIAMOND1 | T1D | 158 (44.3) | ≥25* | — | — | MDI | 24 | 12,699 |
| DSS1 | T1D | 83 (55.4) | 35.1 (14.9) | 27.2 (6.0) | 7.5 (1.1) | MDI | 12 | 4,494 |
| MDEX | T1D | 15 (40.0) | 32.3 (9.3) | 24.7 (2.8) | 7.1 (0.7) | PMP | 6 | 145 |
| NIGHTLIGHT | T1D | 81 (66.3) | 42.3 (11.9) | 29.4 (5.7) | 7.4 (1.0) | CLC, PMP | 32 | 12,329 |
| REPLACE-BG | T1D | 226 (49.6) | 44.0 (13.8) | 27.3 (4.4) | 7.3 (0.7) | PMP | 26 | 36,918 |
| RT-CGM | T1D | 444 (55.0) | 25.1 (15.8) | 23.5 (4.4) | 7.4 (0.9) | MDI, PMP | 26 | 27,938 |
| SENCE | T1D | 144 (50.0) | 5.2 (1.7) | 17.2 (3.0) | 8.3 (0.7) | MDI, PMP | 26 | 14,431 |
| SEVHYPO | T1D | 187 (46.5) | ≥60* | 27.1 (4.7) | 7.7 (1.2) | MDI, PMP | 2 | 1,170 |
| WISDM | T1D | 206 (51.7) | 68.0 (5.7) | 27.1 (4.6) | 7.5 (0.9) | MDI, PMP | 26 | 26,152 |
| DIAMOND2 | T2D | 158 (56.3) | ≥25* | — | — | MDI | 24 | 7,280 |
| NDIAB | Healthy | 163 (68.1) | 31.4 (21.2) | 22.2 (4.2) | 5.2 (0.3) | — | 1.4 | 888 |
| TRIALNET | Healthy | 58 (56.9) | 24.2 (10.9) | 23.9 (5.7) | 5.3 (0.3) | — | 1 | 281 |
| Overall | | 2,462 (52.6) | | | | | | 204,710 |

Statistics are presented as mean (SD) unless otherwise indicated.
T1D denotes type 1 diabetes, T2D type 2 diabetes, BMI body mass index, CGM continuous glucose monitoring, MDI multiple daily injections, PMP insulin pump, CLC closed-loop control.
*Indicates that the data was not available at the subject level and so was taken from the study protocol.

Most (95.9%) of the daily CGM profiles were generated by subjects with T1D treated either by MDI, insulin pump (PMP) or closed-loop control (CLC). Two data sets focused on children with T1D (DCLP5 and SENCE). The DIAMOND2 data set contains data from participants with T2D on MDI treatment and represent 3.6% of the daily CGM profiles generated. The NDIAB and TRIALNET data sets contain data from people without diabetes (healthy). The glycemic control assessed by the mean HbA1c of each participant at baseline ranged between 5.2% for participants without diabetes (NDIAB study) to 9.1% (C_{ITY} study). The healthy people generally have less than 7 days of data, while the people in the vast majority of the other studies have 5 or more weeks of data on average.

### Identifying Clinically-Similar Clusters of Daily CGM Profiles

In one embodiment, for example, the 5 time in ranges are: Level 2 hypoglycemia [T54]: 54 mg/dl, Level 1 hypoglycemia [T70]: from 54 to 69 mg/dL, within Target Range [TIR]: 70 to 180 mg/dL, Level 1 hyperglycemia [T180]: from 180 to 250 mg/dL, and Level 2 hyperglycemia [T250]: above 250 mg/dL, during a 24-hour time period. In other embodiments, depending on the specific clinical metrics being targeted for different populations, other time in ranges can be used (e.g., pregnant women with diabetes where the recommended TIR is 63 to 140 mg/dl). In an aspect of an embodiment of this invention, these time in ranges are used as the input features to the proposed clustering algorithm.

The time in ranges for a single daily CGM profile were used as the input for a single daily CGM profile when performing hierarchical clustering. The input was generated using all daily CGM profiles in the Training data set. The scipy.cluster.hierarchy Python module [35] implementation of hierarchical clustering with the centroid algorithm was used to calculate Euclidean distances between two rows of input. Because we wanted to ensure that the time below range behavior was faithfully captured by the CSCs we weighted the T54 and T70 input columns greater than the TIR, T180 and T250 input columns. Each clinically-similar cluster (CSC) will be a collection of daily CGM profiles such that each daily CGM profile in the collection has essentially the same time in ranges.

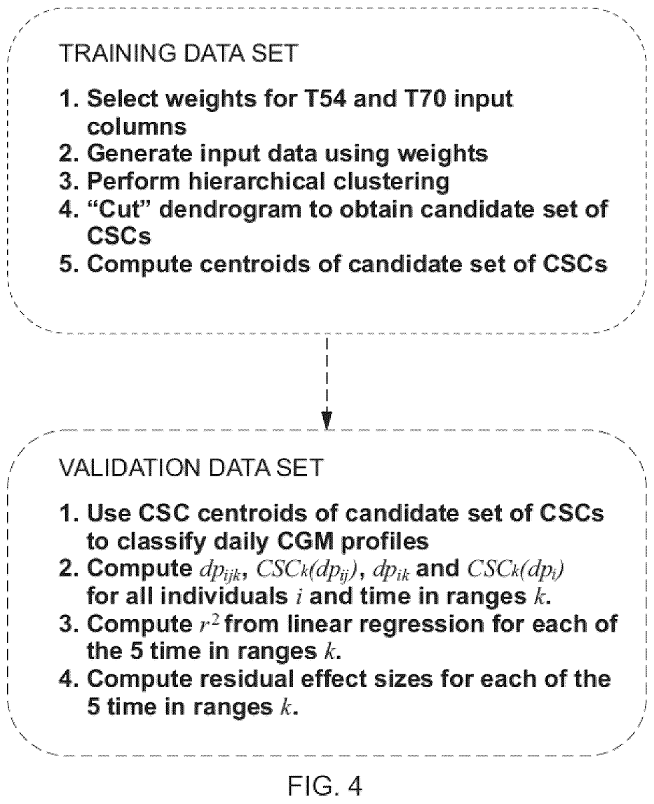

FIG. 4 shows an exemplary process used to identify and then evaluate a single candidate set of CSCs. For a given set of input (determined using the daily CGM profiles in the Training data set and the weights chosen for the T54 and T70 columns), the hierarchical clustering algorithm can produce a dendrogram indicating the hierarchical relationships between the daily CGM profiles in the Training data set. “Cutting” the dendrogram at a specific height can produce a clustering with a specific number of clusters. The evaluation of each candidate set of CSCs can be used the Validation Data Set. The centroids of the CSCs were used to classify each daily CGM profile in the Validation Data Set. Let dpᵢⱼₖ be the time in range k for the j-th daily CGM profile from individual i, and CSCₖ(dpᵢⱼ) be the time in range k for the CSC that the j-th daily CGM profile from individual i is classified as, and where kϵ{T54, T70, TIR, T180, T250}. Furthermore, let

$${dp}_{ik} = \frac{1}{\vert {J}_{i} \vert} {\sum }_{j ∈ {J}_{i}} {dp}_{ijk}$$

$$and let$$

$$C {SC}_{k} ( {dp}_{i} ) = \frac{1}{\vert {J}_{i} \vert} {\sum }_{j ∈ {J}_{i}} C {SC}_{k} ( {dp}_{ij} )$$

where Jᵢ is the set of all daily CGM profiles of individual i. We wanted a single “optimal” set of CSCs which:
1. Maximized the r-squared value of the linear regression through the set of points (dpᵢₖ, CSCₖ(dpᵢ)) for each k, and
2. Ensured that the absolute value of the relative effect size was less than 0.15 for each k, where the relative effect size is computed as the mean of the average differences between dpᵢⱼₖ and CSCₖ(dpᵢⱼₖ) divided by the standard deviation across all dpᵢⱼₖ.

We explored 9 different weightings of the T54 and T70 input columns (i.e., 9 different sets of input to the hierarchical clustering algorithm), which resulted in 166 candidate sets of CSCs being evaluated. The final fixed set of CSCs has 35 clusters. Each CSC is defined by a centroid. The centroid for a given CSC is calculated using the daily CGM profiles in the Training data set which were assigned to the CSC. The centroid of each CSC can be visualized as the CGM-based targets.

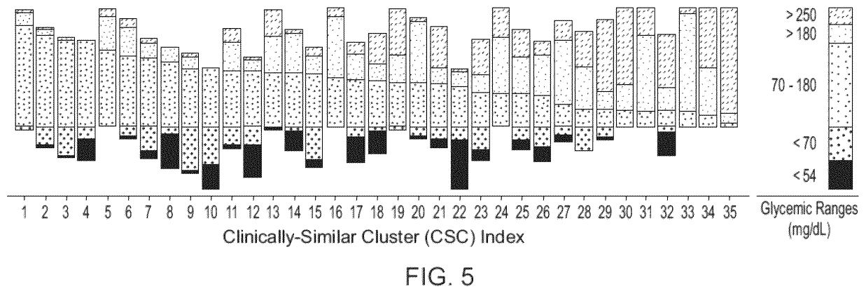

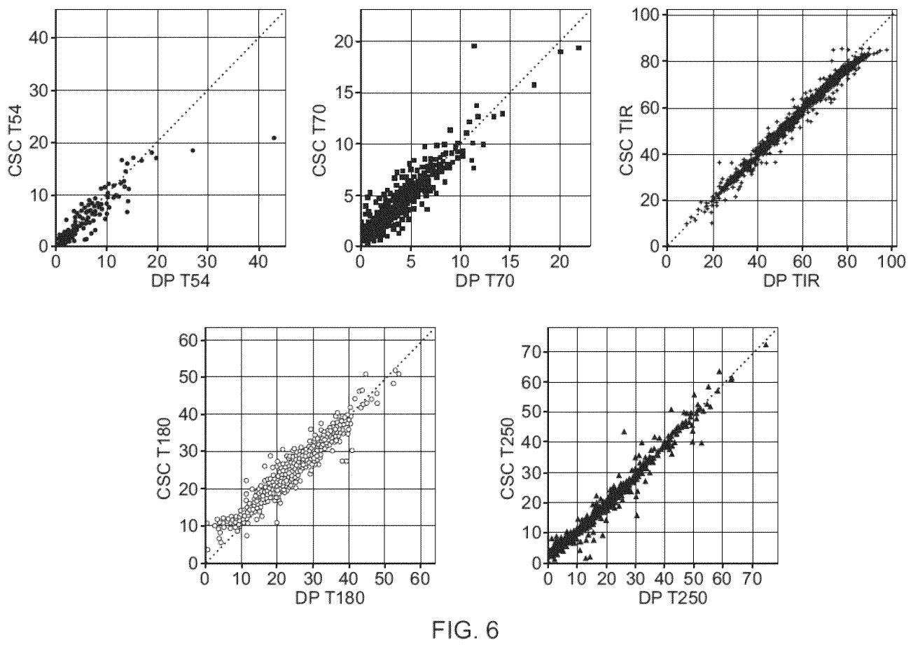

FIG. 5 shows the CGM-based targets visualization associated with each of the 35 CSC centroids. The CSC centroid visualizations in FIG. 5 are ordered by their TIR values (highest on the left to lowest on the right). Inspection of this figure reveals that, as desired, no two CSC visualizations are the same. FIG. 6 plots the scatterplots of the set of points (dpᵢₖ, CSCₖ(dpᵢ)) for each kϵ{T54, T70, TIR, T180, T250}, and Table 2 provides the relative effect sizes for each kϵ{T54, T70, TIR, T180, T250} when the 35 CSCs were used on the set of 141,869 daily CGM profiles from the Testing data set which excludes the 1,169 daily CGM profiles in the testing data set which belong to healthy individuals. These results indicate that the final chosen set of 35 CSCs faithfully represents the clinical characteristics of the daily CGM profiles they are approximating.

TABLE 2
Relative effect sizes and linear regression results for
k ϵ {T54, T70, TIR, T180, T250} when using the daily
CGM profiles from the Testing data set (but excluding
the daily CGM profiles from healthy individuals).

| | Relative Effect | Linear Regression Results | | |
|---|---|---|---|---|
| | Size | Intercept | Slope | r² |
| T54 | −0.073 | 0 | 1.041 | 0.891 |
| T70 | −0.115 | 0 | 0.910 | 0.943 |
| TIR | 0.058 | 0 | 1.026 | 0.999 |
| T180 | −0.047 | 0 | 0.997 | 0.994 |
| T250 | −0.003 | 0 | 1.020 | 0.991 |

Table 3 of the 2019 International Consensus on Time in Range [14] defines guidance for two diabetes groups:
1. Adults with T1D or T2D, and
2. Older/high risk individuals with T1D or T2D.

TABLE 3
Fitted values of the parameters γ, λ, and k in
the modified Weibull equation for the six
different subgroups.

| | | γ | λ | k |
|---|---|---|---|---|
| Health | T1D | 13.400 | 27.251 | 0.632 |
| State | T2D | 9.215 | 15.114 | 0.708 |
| | Healthy | 3.060 | 3.196 | 0.675 |
| T1D | MDI | 14.378 | 23.603 | 0.693 |
| Treatment | Insulin Pump | 13.673 | 31.678 | 0.609 |
| Modality | CLC | 8.281 | 13.509 | 0.686 |

CSC 1 meets the guidance for the adults with T1D or T2D and CSC5 meets the guidance for older/high risk individuals with T1D or T2D. Therefore, physicians have a target CSC for these two situations.

### Tracking Glycemic Volatility:

### Unique CSCs Visited Over Time

Each individual i has a single CGM time series generated during the study they take part in. Note that the CGM time series can have periods during which CGM data is not collected (e.g., during washout periods of the study). Classifying all of the daily CGM profiles in the individual's single time series results in a sequence sᵢ of (possibly non-consecutive) indices indicating the CSC that each daily CGM profile is classified as. Each entry of the sequence sᵢ corresponds to a single day of observation and the days are ordered by date of occurrence. Let fᵢ(t) be the number of unique CSCs visited by individual i after t days of observation. Then fᵢ is the trace of the number of unique CSCs visited by individual i over time and provides an idea of the variability in the individual's blood glucose. In general, a larger number of unique CSCs visited by an individual indicates an increase in blood glucose variability (and thus worse control of blood glucose). Note that in extreme cases it is possible that an individual with large blood glucose variability would only visit a small number of CSCs, and those CSCs visited represent large blood glucose variability.

### The Mean Trace

The mean trace indicates the average behavior of individuals in a subgroup and can help highlight the differences in behavior between different subgroups. For a given subgroup G, the mean value is

$$\overline{{f}_{G}} = \frac{1}{\vert I \vert} {\sum }_{i = 1}^{\vert I \vert} {f}_{i} ( t )$$

where |I| is the total number of individuals in the subgroup. Not all individuals in a subgroup have the same number of days observed in their sequences sᵢ of CSC indices. As such, f̅_̅{̅G̅}̅ is defined only if there is a minimum number of sequences at time t. The minimum number of sequences is a function of the subgroup G.

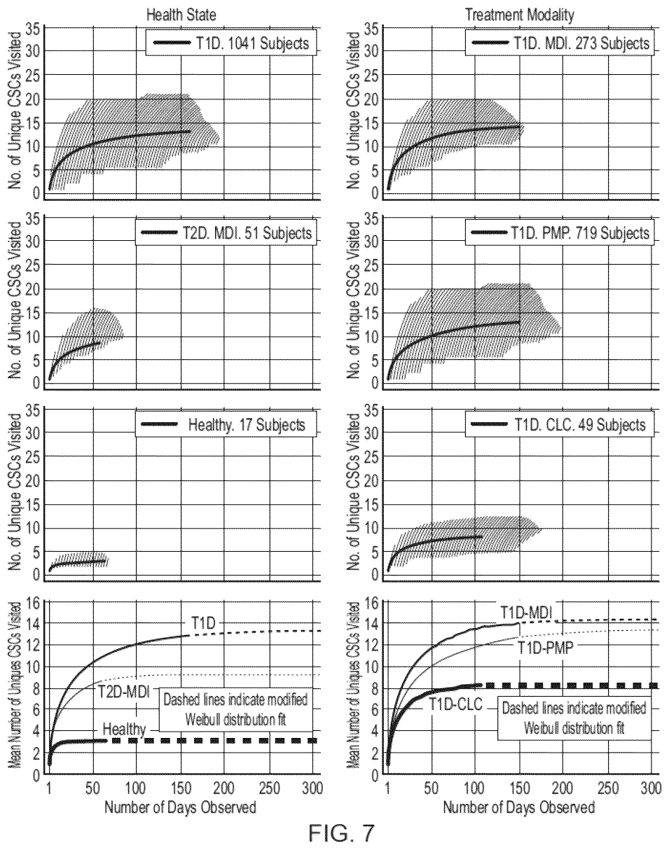

In general we would like to estimate the long-term (more than 2-3 months) behavior of a subgroup, especially for those subgroups lacking longer-term data. To accomplish this, we fit a curve to the mean trace of a subgroup. As seen in the top three rows of plots in FIG. 7, the curves have the general shape of a cumulative density function and we suspected that a Weibull cumulative distribution function (CDF) [36] modified to account for the non-unity upper limit would provide a good fit. The following modified Weibull CDF was used as input to the curve_fit function of the scipy.optimize SciPy module:

$$g ( t ) = γ * ( 1 - {e}^{- {( t / k )}^{λ}} ) .$$

Note that γ modifies the upper limit so that it is no longer 1 as in the Weibull CDF.

FIG. 7 shows the individual, mean, and fitted curves by health state and treatment modality. The gray dashed curves in the plots in the first three rows of FIG. 7 are the individual traces fᵢ while the solid thick lines in the plots in the first three rows of FIG. 7 are the mean traces f̅_̅{̅G̅}̅. The solid thick lines in the plots in the bottom row of FIG. 7 are the mean traces f̅_̅{̅G̅}̅ while the dashed thick lines are the modified Weibull curves fit to each mean trace (the parameters for these fitted curves can be found in Table 3). These curves once again distinguish between both health state and treatment modality because they track glycemic volatility. As expected, on average healthy individuals have the least glycemic volatility, followed by individuals with T2D and then by individuals with T1D. For individuals with T1D, individuals using MDI as the treatment modality on average have the largest glycemic volatility followed by individuals on PMP and then those individuals on CLC.

The fitted modified Weibull curves reach a threshold after approximatively 200 days of observation: the maximum number of unique CSC visited is higher in subjects with T1D than T2D (13.3 CSCs versus 9.2 CSCs) and in subjects treated by MDI versus PMP or CLC (14.4 CSCs versus 13.5 CSCs versus 8.3 CSCs respectively).

### Applications

### Data Structuring and Dimensionality Reduction

The multitude of all possible daily CGM profiles, as clinically represented by AGP and their time in ranges, is reduced to a finite and fixed set of CSCs which can be used as input to decision support, clinical, and automated treatment algorithms.

### Database Indexing

A database indexed by the structure defined by the CSCs can ensure fast and efficient search for subgroups of similar daily CGM profiles. This can enable new features in decision support or automated insulin delivery systems, such as algorithms learning from a person's CGM patterns, and from the patterns of others patients stored in population databases.

### Compression/Encryption of Daily CGM Profiles

Instead of transmitting a daily CGM profile (typically 288 data points), a single number can be transmitted, which identifies the CSC index of the original daily CGM profile. At the receiving end, a decoder equipped with the set of CSCs can reconstruct the AGP clinical characteristics, e.g. the time in ranges and other clinical metrics, of the daily CGM profile with a fidelity that preserves these metrics of the original daily CGM profile.

### Distinguishing Between Health States and Treatment Modalities

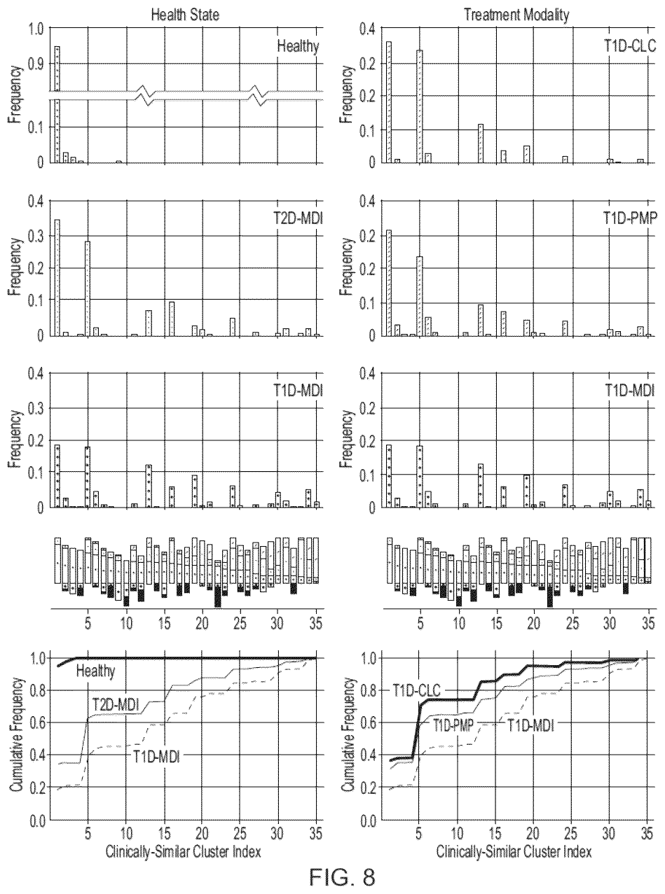

One application of the CSCs is the ability to distinguish between both health states and treatment modalities. FIG. 8 shows the frequency distribution of the 143,036 daily CGM profiles in the testing data set to the 35 different CSCs. The results in these plots are stratified by health state and by treatment modality. It is clear that the frequency distribution is a function of the health state of the individuals being considered (i.e., healthy individuals, or individuals with T1D or T2D). In addition, for individuals with T1D, the frequency distribution is a function of the treatment modality (MDI, PMP, or CLC). As expected, the vast majority (94.6%) of the daily CGM profiles generated by healthy individuals are classified as CSC 1, and over 99% of the daily CGM profiles are classified as one of CSCs 1, 2, or 3. The health state comparison is made between T1D-MDI and T2D-MDI so that the comparison is fair—because the treatment modality is the same, differences in the frequency distribution should be attributable to the difference in diabetes type (T1D versus T2D).

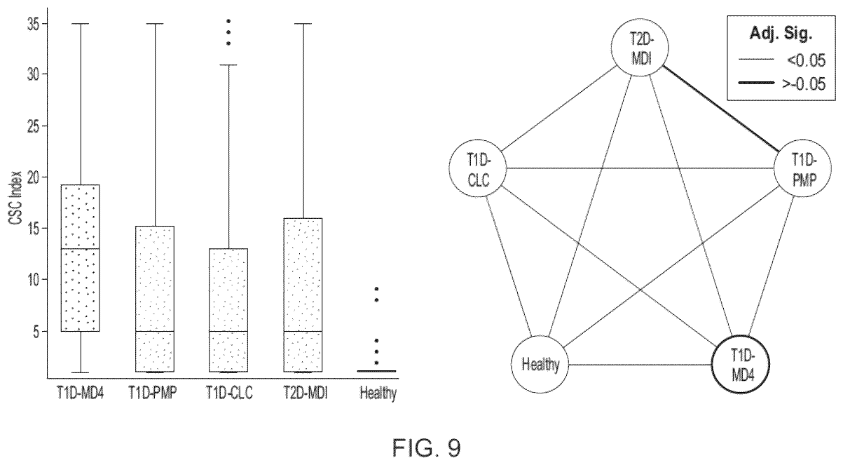

In order to formally test the visual observations, an independent-samples Kruskall-Wallis test was performed between the frequency distributions of the T1D-MDI, T1D-PMP, T1D-CLC, T2D-MDI, and Healthy subgroups, resulting in P<0.001. Pairwise comparisons with Bonferroni correction adjustment for multiple tests revealed adjusted significance of P<0.05 for all pairwise comparison except for that between T1D-PMP and T2D-MDI (see FIG. 9), indicating that pump therapy brings the clinical treatment outcomes of T1D-MDI patients close to the clinical outcomes in T2D, while the clinical outcomes in T1D-CLC are superior to the clinical outcomes in T2D.

### CGM Replacement for Common Clinical Tests

Measuring fasting glucose level, homeostatic (HOMA) assessment of insulin sensitivity and beta cell function, or Oral Glucose tolerance test (OGTT) are common clinical methods for the evaluation of the glycemic health state of a person. These, and other, common glycemic function tests typically require a physician visit, blood draws, and laboratory analysis. In the case of OGTT, several hours of testing are needed in a clinical setting. While cumbersome, these tests are required routinely in many situations, e.g. frequent OGTT in gestational diabetes. The ability of CSCs to distinguish with high fidelity between health states could serve as a replacement of these clinical tests—a 10-day CGM wear in home environment, accompanied by a predefined schedule of meals and physical activity, will achieve diagnostic results similar to those accepted in the clinical practice, while greatly simplifying the data collection.

### CGM Pattern Recognition and Forecast

The transition probability matrix describing the evolution of a patient across the predefined CSCs, is a natural tool for observation of disease or treatment progression. Pattern recognition, or recurrent behaviors, are reflected by patterns, or cycles, detected in the transition probabilities from one state to the next. Short- or long-term forecasts of glycemic control are based on probability patterns or recurrent visits to a certain subspace of the Markov chain state space. The latter is a subject of the theory of semi-Markov chains, which result from aggregation (lumping) of the state space into relevant subsets, characterized by random duration of time spent in each subset.

### Tracking Disease Progression Over Time

Disease deterioration is signified by transitions into undesired states and, conversely, a successful treatment optimization of medication titration is reflected by transition into clinically desirable states. In a practical application, the state space of the Markov chain is defined/aggregated to correspond to the CSCs defined by an aspect of an embodiment of the present invention system, method and computer readable medium.

### Example 2

The ability to track blood glucose has evolved from episodic self-monitoring to contemporary continuous glucose monitoring (CGM). Clinical decisions based on CGM, however, are difficult because CGM yields voluminous and complex data sets which require advanced analyses to provide insight. This work presents a two-step iterative process which reduces the “clinical dimensionality” of the CGM data space by identifying a fixed set of clinically-similar clusters (CSCs), such that the daily CGM profiles within each cluster convey a similar clinical message. The two-step process uses hierarchical clustering on a Training data set to identify candidate sets of CSCs, and linear regression and relative effect sizes to evaluate the ability of the candidate set of CSCs to capture five different times in ranges of daily CGM profiles from a Validation data set. The optimal set of 35 CSCs identified using the Validation data set was then used to classify the daily CGM profiles in a separate Testing data set. The results indicate that the set of CSCs is robust, generalizes well, but most importantly captures the clinical characteristics of a daily CGM profile with high fidelity. This fixed set of CSCs enable an individual's daily glycemic control over time to be tracked, facilitate the design of personalized treatments, and potentially enable automated treatment optimization by predefined rules mapping an optimal treatment response to each CSC. The CSCs can also be used to visualize differences in glycemic control between individuals and differences between treatment modalities, identifying individuals who might benefit from treatment adjustment.

Glucose variability (GV) in diabetes reflects an underlying bio-behavioral process of blood glucose (BG) fluctuation that has two principal dimensions: amplitude reflecting the extent of BG excursion, and time reflecting the frequency of BG variation and the rate of event progression. Observation of this process has evolved from episodic self-monitoring which generates a few BG readings each day to contemporary continuous glucose monitoring (CGM), which generates large data sets, time series of glucose readings, that are equally spaced in time (e.g., every 5 minutes). The increasing proliferation of CGM technologies inevitably creates vast amounts of data. The CGM time series data are used to derive insights, which allow for better treatment of diabetes, including risk stratification, prediction of events of interest (e.g., impending hypoglycemia or hyperglycemia), or automated closed-loop control commonly referred to as the “artificial pancreas”. To improve the clinical utility of CGM data and simplify their interpretation, the 2019 International Consensus on Time in Range (TIR) proposed TIR as a primary CGM-based metric of glycemic control and set clinical targets for its use, and this “TIR system of metrics” has been widely adopted over the past 3 years. The TIR system is based on the Ambulatory Glucose Profile (AGP), introduced as a template for data presentation and visualization. Originally proposed by Mazze et al. for episodic self-monitoring data, the standardized CGM report incorporates core CGM metrics and targets along with a 14-day composite glucose profile as an integral component of clinical decision making.

One area of the existing literature focuses on using CGM data to cluster subjects (e.g., cluster a subject into the group with higher risk for gestational diabetes mellitus), or to build classification models (e.g., classify a subject as healthy, pre-diabetic, or diabetic based on their CGM data). Acciaroli et al. used 25 CGM-based glycemic variability indices as inputs to a 2-step binary logistic regression model. The model first classifies subjects as healthy or not healthy, and then classifies those subjects who were not classified as healthy in the first step as either affected by impaired glucose tolerance (IGT) or type 2 diabetes (T2D). The model was able to distinguish between healthy and those with IGT or T2D, and also between IGT and T2D. Bartolome et al. developed an algorithm they named GlucoMine with the aim of uncovering individualized patterns in longer-term CGM data (3-6 months of data) which are not apparent in shorter-term data. Gecili et al. used functional data analysis to identify phenotypes of glycemic variation in type 1 diabetes (T1D) using CGM data. They conclude that these phenotypes can be used to optimize T1D management for subgroups of subjects who are at highest risk for adverse outcomes. Inayama et al. derived summary statistics from CGM data and then used hierarchical clustering of the summary statistics to cluster 29 women into three groups (low glucose levels with less glucose variability, L; moderate glucose levels with moderate-to-high glucose variability, M; high glucose levels with high glucose variability, H). They found that women with gestational diabetes mellitus (GDM) tend to be in the H group, and thus their clustering can help identify subgroups of women with characteristics of GDM. Li et al. took CGM time series data and decomposed it into trend, seasonal (daily), and random components. The trend components were then clustered using k-means clustering which resulted in 5 clusters, 2 of which had increasing trends, 2 of which had decreasing trends, and 1 of which had no trend (unchanged). They then used these five clusters to group subjects into 3 groups: an increasing group, a decreasing group, and an unchanged group. The fasting plasma glucose values of subjects assigned to the increasing and decreasing groups increased and decreased respectively following a 6-month long glucose-lowering treatment. Tao et al. clustered 24-hour CGM time series generated by T2D subjects with the goal of identifying subjects with different degrees of dysglycemia and clinical phenotypes. Mao et al. developed a pipeline for analysis of CGM data with the goal of identifying glucotypes: groups of subjects where the subjects differ in their degree of control, amount of time spent in range, and presence and timing of hyper- and hypoglycemia. They state that, in addition to other biometric data, their method “can be utilized to guide targeted interventions among patients with diabetes”.

Other literature uses CGM data to build a framework for analysis which can then be used in a number of different applications (e.g., identifying different sub-types of patients). Hall et al. developed an “analytical framework that can group individuals according to specific patterns of glycemic responses called ‘glucotypes’ that reveal heterogeneity, or subphenotypes, within traditional diagnostic categories of glucose regulation”. Matabeuna et al. used CGM data to derive glucodensities, where a glucodensity is the distribution of blood glucose values from the CGM time series of an individual subject with the claim that the glucodensity is an extension of the time-in-range metric. They suggest that glucodensities could be used in clinical practice to provide a “more accurate representation of the glycemic profile of an individual”, “identify different subtypes of patients based on their glycemic condition and other variables”, and even “establish if there are statistically significant differences between patients subjected to different interventions”.

All papers referenced above have the end objective of classifying individuals with diabetes into distinct subgroups, and none of the classifications capture the daily variation of glycemic control within an individual. To more comprehensively utilize the timing structure of CGM data, we defined daily CGM profiles, 24-hour CGM time series, and used them to establish a finite set of 483 representative daily profiles or motifs, which they claim can be matched to almost any daily CGM profile. The set of motifs was externally validated and can be considered fixed. The motifs reflect not only differences between individuals, but also differences in day-to-day variation in glycemic control of an individual.

The work proposed in this paper builds an analysis framework based on the “TIR system of metrics” that enables classification of the daily glycemic behavior of an individual. This classification of a single day provides the base for a large number of different analyses: it can be used to define a fixed number of groups if desired, but can also be used to track individuals across time for modeling purposes, for clinical subgroup stratification and transitions from one subgroup to another, or for informing automated control strategies, to name a few. In addition, while the existing literature assumes that the subject has a specific type of diabetes, the analysis framework presented in this paper is agnostic to the type of diabetes, and works equally well with CGM data generated by individuals in health—the observed patterns identify the glycemic states of an individual without the need for prior classification or diagnosis.

The data and methods used to identify the clinically-similar clusters (CSCs) is outlined herein, which includes use of the methods to obtain a fixed set of CSCs using training and validation data sets, and then evaluating the performance of the fixed set of clusters on a separate testing set.

### Methods

Methods involve: training, validation, and testing data sets; defining the input generation for the hierarchical clustering methods used to identify a candidate set of clinically-similar clusters (CSCs); a two-step, iterative process used to identify the “optimal” set of CSCs.

### Data Set Sources

The data used in this work came from:
1. The University of Virginia's Center for Diabetes Technology (namely the DCLP1 [4], DCLP3 [5], DIA1 [21], DIA2 [22], DSS1 [23], NTLT [24], and TRLNT studies), and
2. The Jaeb Center for Health Research website¹ (namely the CITY [25], DCLP5 [6], NDIAB [26], MDEX [27], REPBG [28], RTCGM [29, 30], SENCE [31], SEVHYPO [32], and WISDM [33, 34] studies). For these studies the analyses, content, and conclusions presented in this work are solely the responsibility of the authors and have not been reviewed by any of the study groups. 1https://public.jaeb.org/datasets/diabetes

From each of these studies, the procedure outlined in our previous paper [19] was used to process the CGM time series and define the daily CGM profiles, where a daily CGM profile is a time series of 288 blood glucose data points collected every 5 minutes during the midnight-to-midnight (24-hour) period. From the 16 data sets, there were 2,462 subjects and a total of 204,710 daily CGM profiles. The studies represent healthy individuals, individuals with T1D, and individuals with T2D. The studies also represent a variety of treatment modalities including multiple daily injections (MDI), insulin pump (CSII), and closed loop control (CLC). The characteristics of the participants in each study are detailed in Table 4. Most (95.9%) of the daily CGM profiles were generated by subjects with T1D treated either by MDI, CSII or CLC.

Two data sets (DCLP5 and SENCE) focused on children with T1D. The Dia2 data set contains data from participants with T2D on MDI treatment and represent 3.6% of the daily CGM profiles generated. The NDIAB and TRLNT data sets contain data from people without diabetes (healthy). The glycemic control assessed by the mean HbA1c of each participant at baseline ranged between 5.2% for participants without diabetes (NDIAB study) to 9.1% (City study). The healthy people generally have less than 7 days of data, while the people in the vast majority of the other studies have 5 or more weeks of data on average.

The 204,710 daily CGM profiles were used to form three different data sets, each with a distinct purpose: The Training Data Set: This data set was composed of 23,916 daily CGM profiles taken from the DCLP1, DCLP3, DIA1, DIA2, DSS1, and NTT studies and was used to define the candidate sets of CSCs.

TABLE 4
Characteristics of the 16 data sets used in this work.

| | | No. Study | | | | | Study | No. Daily |
|---|---|---|---|---|---|---|---|---|
| Study | Health | Subjects | Age, | BMI, | HbA1c, | Treatment | Dur., | CGM |
| Name | State | (% Female) | years | kg/m² | % | Modalities | weeks | Profiles |
| CITY | T1D | 153 (49.7) | 17.5 (2.9) | 25.4 (5.0) | 9.1 (1.0) | MDI, CSII | 26 | 20,226 |
| D_{CLP}1 | T1D | 125 (47.2) | 32.5 (14.9) | 26.7 (5.2) | 7.5 (0.9) | CLC, CSII | 13 | 5,459 |
| D_{CLP}3 | T1D | 168 (50.0) | 32.8 (16.0) | 25.8 (5.3) | 7.6 (1.0) | CLC, CSII | 26 | 24,450 |
| D_{CLP}5 | T1D | 93 (50.5) | 10.7 (2.0) | 19.4 (4.0) | 7.7 (1.1) | CLC, CSII | 16 | 10,050 |
| DIA1 | T1D | 158 (44.3) | ≥25* | — | — | MDI | 24 | 12,699 |
| DSS1 | T1D | 83 (55.4) | 35.1 (14.9) | 27.2 (6.0) | 7.5 (1.1) | MDI | 12 | 4,294 |
| MDEX | T1D | 15 (40.0) | 32.3 (9.3) | 24.7 (2.8) | 7.1 (0.7) | CSII | 6 | 145 |
| NTLT | T1D | 81 (66.2) | 42.2 (11.9) | 29.4 (5.7) | 7.4 (1.0) | CLC, CSII | 32 | 12,329 |
| REPBG | T1D | 226 (49.6) | 44.0 (13.8) | 27.3 (4.4) | 7.3 (0.7) | CSII | 26 | 36,918 |
| RTCGM | T1D | 444 (55.0) | 25.1 (15.8) | 23.5 (4.4) | 7.4 (0.9) | MDI, CSII | 26 | 27,938 |
| SENCE | T1D | 144 (50.0) | 5.2 (1.7) | 17.2 (3.0) | 8.3 (0.7) | MDI, CSII | 26 | 14,431 |
| SEVHYPO | T1D | 187 (46.5) | ≥60* | 27.1 (4.7) | 7.7 (1.2) | MDI, CSII | 2 | 1,170 |
| WISDM | T1D | 206 (51.7) | 68.0 (5.7) | 27.1 (4.6) | 7.5 (0.9) | MDI, CSII | 26 | 26,152 |
| DIA2 | T2D | 158 (56.3) | ≥25* | — | — | MDI | 24 | 7,280 |
| NDIAB | Healthy | 163 (68.1) | 31.4 (21.2) | 22.2 (4.2) | 5.2 (0.3) | — | 1.4 | 888 |
| TRLNT | Healthy | 58 (56.9) | 24.2 (10.9) | 23.9 (5.7) | 5.3 (0.3) | — | 1 | 281 |
| Overall | | 2,462 (52.6) | | | | | | 204,710 |

Statistics are presented as mean (SD) unless otherwise indicated.
T1D denotes type 1 diabetes, T2D type 2 diabetes, BMI body mass index, CGM continuous glucose monitoring, MDI multiple daily injections, CSII insulin pump, CLC closed-loop control.
*Indicates that the data was not available at the subject level and so was taken from the study protocol.

1. The Validation data set: This data set was composed of 37,758 daily CGM profiles again taken from the DCLP1, DCLP3, DIA1, DSS1, and NTLT studies and was used to a) assess the performance of each candidate set of CSCs, and b) select the final and fixed set of CSCs.
2. The Testing Data Set: This data set was composed of 143,036 daily CGM profiles taken from the City, DCLP5, DIA2, M_{DEX}, NDIAB, REPBG, RTCGM, SENCE, SEVHYPO, TRLNT and WISDM studies and was used to evaluate the robustness and generalizability of the final selected set of CSCs.

Hierarchical clustering to identify clinically-similar cluster of daily CGM profiles.

The TIR system of metrics defines 5 times in ranges for blood glucose values, namely
1. Level 2 hypoglycemia (T54): blood glucose strictly less than 54 mg/dl,
2. Level 1 hypoglycemia (T70): blood glucose greater than or equal to 54 mg/dL and strictly less than 70 mg/dL,
3. Target range (TIR): blood glucose greater than or equal to 70 mg/dL and less than or equal to 180 mg/dL,
4. Level 1 hyperglycemia (T180): blood glucose strictly greater than 180 mg/dL and less than or equal to 250 mg/dL, and
5. Level 2 hyperglycemia (T250): blood glucose strictly greater than 250 mg/dL.

The 5 times in ranges for each daily CGM profile were used as input for the hierarchical clustering, which was computed with the scipy.cluster.hierarchy Python module [35], where the centroid method was used to calculate Euclidean distances between two rows of input. The T54 and T70 input columns were multiplied by weights ω_{T54}≥1 and ψ_{T70}≥1 to emphasize the importance of these two input columns during the clustering process, and to ensure that the time below range behavior (i.e., T70 and T54) is captured in great fidelity. Each clinically-similar cluster is a collection of daily CGM profiles such that each daily CGM profile in the collection has essentially the same times in ranges. The centroid of each cluster of daily CGM profiles identified by the hierarchical clustering can define the CSC. In contrast to our previously published work with daily CGM profiles (see [19, 20]), the CSCs ignore the within-day timing of glycemic variation. Thus, the reduction of a daily CGM profile composed of 288 data points to a single CSC centroid composed of just 5 data points, one for each of the 5 times in ranges, involves abstracting away the timing information contained within a daily CGM profile.

### An Iterative Procedure to Identify the “Optimal” Set of CSCs

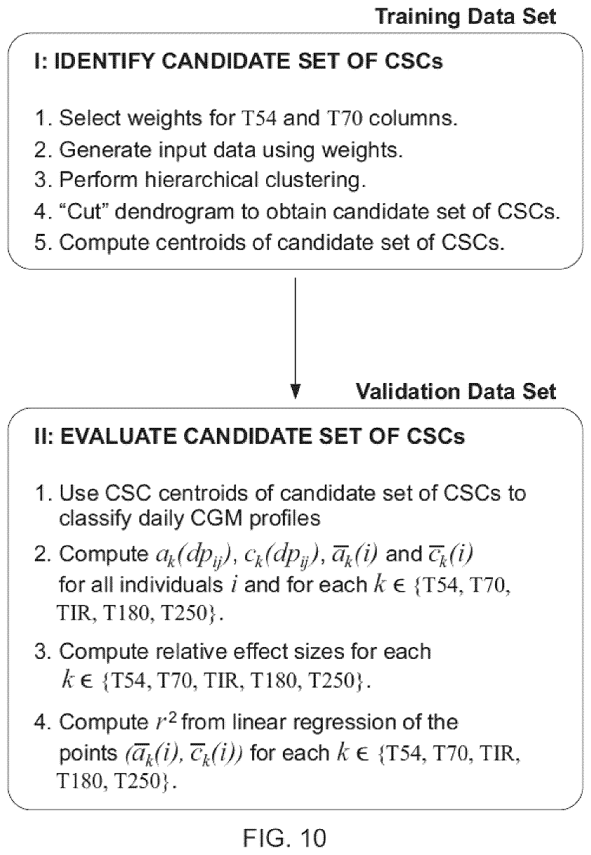

FIG. 10 shows an exemplary two-step, iterative process used to identify the “optimal” set of CSCs. The input used to define a candidate set of CSCs was generated using the 23,916 daily CGM profiles in the Training data set. For a given set of input (determined using the daily CGM profiles in the Training data set and the weights chosen for the T70 and T54 columns), the hierarchical clustering algorithm can produce a dendrogram indicating the hierarchical relationships between the times in ranges of the daily CGM profiles in the Training data set. “Cutting” the dendrogram at a specific height can define a specific set of N clusters, and the centroid of each cluster is calculated using the daily CGM profiles assigned to that cluster. This set of N clusters is a candidate set of CSCs which must then be evaluated.

The evaluation of each candidate set of CSCs begins by classifying the 37,758 daily CGM profiles in the Validation Data Set using the CSC centroids. Let aₖ(dpᵢⱼ) be the time in range k of the j-th daily CGM profile from individual i, and cₖ(dpᵢⱼ) be the time in range k of the centroid of the CSC that the j-th daily CGM profile from individual i is classified as, where kϵ{T54, T70, TIR, T180, T250}. Furthermore, let

$${\overline{a}}_{k} ( i ) = \frac{1}{\vert {J}_{i} \vert} {\sum }_{j ∈ {J}_{i}} {a}_{k} ( {dp}_{ij} ) \qquad ( 1 )$$

$$and let$$

$${\overline{c}}_{k} ( i ) = \frac{1}{\vert {J}_{i} \vert} {\sum }_{j ∈ {J}_{i}} {c}_{k} ( {dp}_{ij} ) \qquad ( 2 )$$

where Jᵢ is the set of all daily CGM profiles of individual i. Given the above definitions, the “optimal” set of CSCs will
1. Have a relative effect size that is less than or equal to δ for each kϵ{T54,T70,TIR,T180,T250}, and
2. Maximize the r² value of the linear regression with intercept through the set of points (āₖ(i), c̅ₖ(i)) for each kϵ{T54,T70,TIR,T180,T250}.

The relative effect size is computed as

$$\frac{\frac{1}{\vert I \vert} {\sum }_{i ∈ I} [ \frac{1}{\vert {J}_{i} \vert} {\sum }_{j ∈ {J}_{i}} ( {a}_{k} ( {dp}_{ij} ) - {c}_{k} ( {dp}_{ij} ) ) ]}{S D ( {a}_{k} ( {dp}_{ij} ) ) ∀ i ∈ I , j ∈ J} \qquad ( 3 )$$

where the denominator is the standard deviation of all 37,758 aₖ(dpᵢⱼ) values. Linear regression with an intercept (specifically the scipy.stats.linregress function from the SciPy [35] Python package) was used during this part of the process so that r² values could be compared.

TABLE 5
Relative effect size and linear regression results for the
five different times in ranges {T54, T70, TIR, T180, T250}
when the 35 CSCs in Ψ are used to classify the 37,758 daily
CGM profiles in the Validation data set (top) and the
143,036 daily CGM profiles in the Testing data set
(bottom).

| | | Relative | Linear Regression | |
|---|---|---|---|---|
| Data Set | Range | Effect Size | Slope | r² |
| Validation | T54 | −0.124 | 0.918 | 0.870 |
| | T70 | −0.143 | 0.885 | 0.876 |
| | TIR | 0.064 | 1.025 | 0.992 |
| | T180 | −0.037 | 0.983 | 0.955 |
| | T250 | −0.012 | 1.017 | 0.987 |
| Testing | T54 | −0.080 | 1.037 | 0.860 |
| | T70 | −0.129 | 0.908 | 0.840 |
| | TIR | 0.116 | 1.054 | 0.965 |
| | T180 | −0.124 | 0.957 | 0.862 |
| | T250 | −0.011 | 1.019 | 0.981 |

Note
that the linear regression results presented are for linear regression with a fixed intercept of 0.

In this application the relative effect size 8=0.15 is an attempt to ensure that a) hypoglycemia events which are relatively uncommon are captured with reasonably high fidelity, i.e., the swap of a daily CGM profile with CSC does not result in more than 15% deviation, and b) the set of CSCs captures the glucose dynamics regardless of the individual generating the daily CGM profiles.

### Results

The “optimal” set of clinically-similar clusters is identified and the performance of this set on the Testing data set of daily CGM profiles is also presented.

### Identification of the Set of Clinically-Similar Clusters

Nine different combinations of weights (ω_{T54}, ω_{T170}) were explored. An upper bound of 60 and a lower bound of 15 limited the number of candidate sets of CSCs considered: any “cut” of the hierarchical clustering dendrogram that resulted in a candidate set of CSCs with more than 60 or less than 15 CSCs was not evaluated using the second step of the iterative procedure. There were 166 candidate sets of CSCs evaluated and the final chosen (fixed) set of CSCs, Ψ, has 35 clusters. The centroid of a given CSC is calculated using the daily CGM profiles in the Training data set which were assigned to that CSC. The relative effect size and linear regression results from evaluating Ψ using the 37,758 daily CGM profiles of the Validation data set are presented in the top half of Table 5. Note that the linear regression results are for linear regression with a fixed intercept of 0, accomplished using the sklearn.linear model.LinearRegression function of the scikit-learn Python package. Scatter plots of the set of points (āₖ(i), c̅ₖ(i)) for each kϵ{T54,T70,TIR,T180,T250} when Ψ were used to classify the 37,758 daily CGM profiles in the Validation data set. These results demonstrate that Ψ, the final chosen set of 35 CSCs, faithfully represents the clinical characteristics of the daily CGM profiles being approximated. In particular, the CSCs capture the relatively uncommon hypoglycemia components of daily CGM profiles with high fidelity.

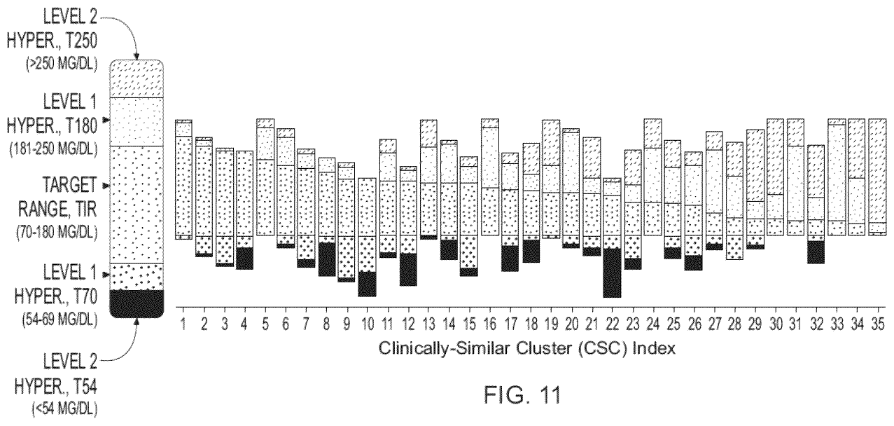

The centroid of a CSC can be visualized as the CGM-based targets, and are reproduced in the legend in FIG. 11. The height of each color in the visualization corresponds to the percentage of time that the centroid spends in each range. FIG. 11 shows the CGM-based targets visualization associated with each one of the 35 CSC centroids in Ψ ordered by their TIR values (highest on the left to lowest on the right). Inspection of this figure reveals that, as desired, no two CSCs are the same because no two CSC centroid visualizations are the same. In addition, further inspection reveals that the majority of the centroids have significant hypoglycemia (T54 and T70) components to them. This is a consequence of the weighting of the T54 and T70 input columns when generating candidate sets of CSCs in the first step of the iterative procedure.

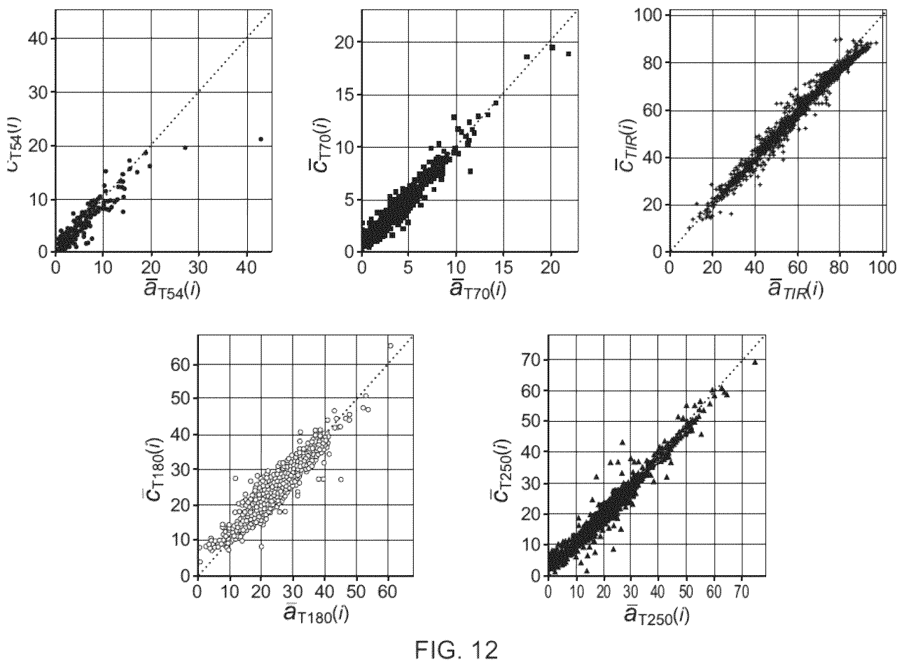

The centroids of the 35 CSCs in Ψ were then used to classify the 143,036 daily CGM profiles in the Testing data set. FIG. 12 plots the set of points (āₖ(i), c̅ₖ(i)) for each kϵ{T54,T70,TIR,T180,T250}, while the bottom half of Table 5 provides the associated linear regression results and relative effect sizes. These results demonstrate that Ψ is robust and generalizes to data that it has not seen previously and, in the case of the healthy individuals, was not trained on.

### Visualizing Individual Glycemic Control

The CSCs can be used to visualize differences in glycemic control between individuals who have the same health state and treatment modality, and thus identify individuals who may need more personalized attention. Let

$$= \frac{{u}_{i}}{k} \qquad ( 4 )$$

where uᵢ is the number of unique CSCs that are needed to classify k daily CGM profiles of individual i. Because the total number of unique CSCs is bounded (i.e., there are just 35 different CSCs), if k is large, then ûᵢ will tend to 0. To overcome this issue k was fixed at 28 daily profiles (i.e., 4 weeks of data). Then ūᵢ is defined as the average of the values ûᵢ generated by computing each ūᵢ using a sliding window of k=28 daily CGM profiles, where the sliding window advances by 7 days (1 week of data), and where each sliding window must have at least 14 daily CGM profiles (2 weeks of data) for ûᵢ to be computed. For example, if an individual i has 40 daily CGM profiles, ûᵢ will be the mean of ûᵢ₁, ûᵢ₂, ûᵢ₃, and ûᵢ₄ where ûᵢ₁ is computed using daily CGM profiles 1 through 28, ûᵢ₂ is computed using daily CGM profiles 8 through 35, ûᵢ₃ is computed using daily CGM profiles 15 through 40, and ûᵢ₄ is computed using daily CGM profiles 22 through 40. Note that if the number of daily CGM profiles in the sliding window is less than k (as in the sliding window for ûᵢ₄ which only has 18 daily CGM profiles), then the denominator in Equation (4) is set to the number of daily CGM profiles in the sliding window. Because the different CSCs are distinct (i.e., by construction no two CSC centroids have the same times in ranges), ûᵢ is a measure of how volatile the day-to-day glycemic control of the individual is. Values of ûᵢ close to 1/k indicate that the individual does not visit a large number of different CSCs, and consequently the individual does not have high volatility in their day-to-day glycemic control, while values of ûᵢ much greater than 1/k indicate that the individual has higher volatility in their day-to-day glycemic control.

Let mᵢ be the average CSC index of k daily CGM profiles of individual i, and let m̅ᵢ be the average of the values mᵢ generated by computing each mᵢ using a sliding window of k=28 daily CGM profiles, where the sliding window advances by 7 days and where each sliding window must have at least 14 daily CGM profiles for m̅ᵢ to be computed. Because the CSCs are indexed such that the centroid of CSC1 has the highest TIR value while the centroid of CSC35 has the lowest TIR value, m̅ᵢ is a measure of the daily glycemic control of individual i. Values of m̅ᵢ close to 1 indicate that the individual spends more time in target range, while values close to 35 indicate that the individual spend less time in target range.

FIG. 12 shows exemplary scatterplots of the points (a̅ₖ̅(i), c̅ₖ̅(i)) for kϵ{T54, T70, TIR, T180, T250}which result from using Ψ to classify the 141,867 daily CGM profiles of the Testing data set.

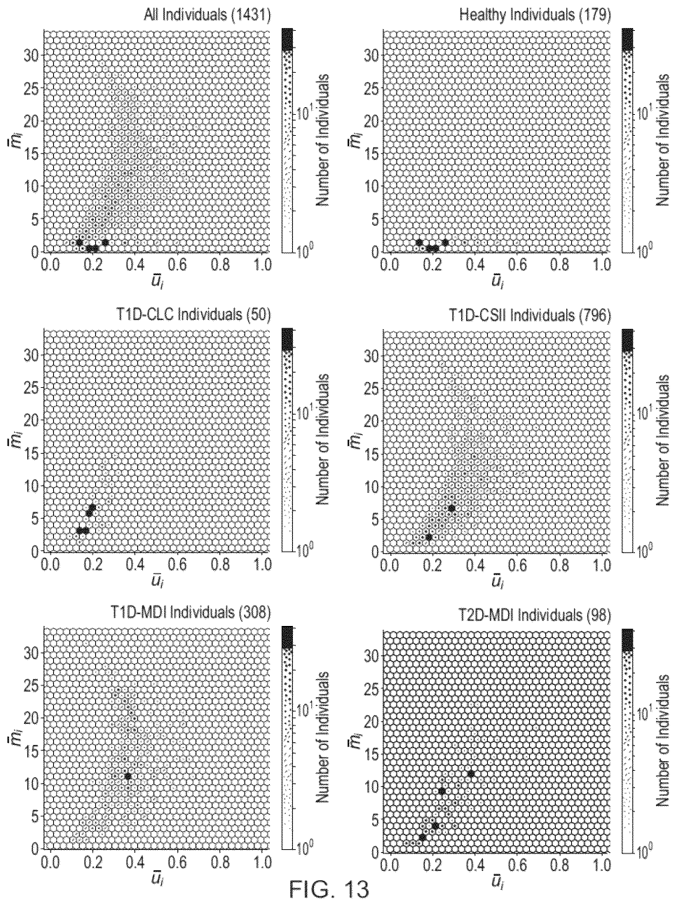

FIG. 13 plots hexbins, 2D histogram plots “in which the bins are hexagons and the color represents the number of data points within each bin”, for the points (ūᵢ, m̅ᵢ). In the case of individuals with T1D or T2D, the point (ûᵢ, m̅ᵢ) was only generated if |J_{i|≥}14, i.e., if individual i had at least 14 daily CGM profiles. This constraint was relaxed to |Jᵢ|≥4 for healthy individuals because in general these individuals did not have more than 7 daily CGM profiles (see the NDIAB and TRLNT rows)—in this case ūᵢ and m̅ᵢ were computed using just the single sliding window.

The top left plot in FIG. 13 plots the points (ūᵢ, m̅ᵢ) for all individuals and min value again strates the wide range of glycemic control that is exhibited by individuals. The other plots which plot the points (ūᵢ, m̅ᵢ) for individuals with different health state and treatment modality combinations further illustrate this point: even when a single health state and treatment modality combination are being considered, there is still a wide variety of glycemic control. As expected the points (ūᵢ, m̅ᵢ) for healthy individuals are located toward the bottom left hand side of the plot. The larger ūᵢ values are due to the small number of days of CGM data which are available for the healthy individuals; in general, we would expect the points (ūᵢ, m̅ᵢ) of healthy individuals to be located toward the bottom left hand corner of the plot. The difference in plots suggests that, as expected, in general individuals with T1D on CLC therapy have better glycemic control than individuals with T1D on CSII therapy, who in turn have better glycemic control than individuals with T1D on MDI therapy.

Note that if ūᵢ is small but m̅ᵢ is large, then the individual does not visit a large number of different CSCs and does not spend a lot of time in target range. This is a worst case scenario, and is realized by the individual in the upper left hand corner of the ‘T1D-Individuals’ plot. Further analysis indicates that this individual spends 89% of their 74 daily CGM profiles in a CSC with index greater than or equal to 30, and 53% of the 74 daily CGM profiles are classified as CSC 35. This individual is a prime example of someone who might benefit from more personalized clinical attention.

In this work we have presented a two-step iterative process to identify a fixed set of clinically-similar clusters (CSCs) of daily CGM profiles. The two-step process uses hierarchical clustering on a Training data set to identify candidate sets of CSCs, and a Validation data set to evaluate the performance of the candidate set of CSCs. In particular, the ability of the CSCs to faithfully capture the five different times in ranges of the daily CGM profiles being classified is evaluated. The fixed set of 35 CSCs, Ψ, was then used to classify the daily CGM profiles in a separate Testing data set, and the results indicated that the set is robust and generalizes well. In addition, the distribution of daily CGM profiles to the different CSCs is shown to be specific to health state and treatment modality.

There are a variety of possible applications of the CSCs. The multitude of all daily CGM profiles, clinically represented by their times in ranges, is reduced to a finite and fixed set of CSCs which can be used as input to decision support, clinical, and automated treatment algorithms. Furthermore, a database which is indexed by the structure defined by the CSCs will ensure fast and efficient search for subgroups of clinically similar daily CGM profiles. The CSCs also allow changes in an individual's daily glycemic control to be tracked over time which has the potential to enable new features in decision support, or inform automated insulin delivery systems, such as algorithms learning from a person's CGM patterns, and from the patterns of others patients stored in population databases. Finally, the abstraction of the typical 288 data points of a daily CGM profile to a single CSC index is both a compression and encryption of the data. These applications are the subject of further research.

The following 8 data sets were downloaded from https://public.jaeb.org/datasets/diabetes: CITY, DCLP5, MDEX, REPBG, RTCGM, SENCE, SEVHYPO, WISDM. The analyses, content, and conclusions presented in this work are solely the responsibility of the authors and have not been reviewed by any of the study groups (CITY: CGM Intervention in teens and Young Adults with T1D Study Group; DCLP5: iDCL Trial Research Group; MDEX: T1D Exchange Mini-Dose Glucagon Exercise Study Group; REPBG: REPLACEBG Study Group; RTCGM: JDRF CGM Study Group; Sence: Strategies to Enhance New CGM Use in Early Childhood Study Group; SEVHYPO: T1D Exchange Severe Hypoglycemia in Older Adults with Type 1 Diabetes Study Group; WISDM: WISDM Study Group).

TABLE 6
T54 and T70 weight combinations—The
nine different combinations of
weights ω_{T54} and ω_{T70} used to emphasize
the T54 and T70 input columns when
performing hierarchical clustering

| Combination | | |
|---|---|---|
| No. | ω_{T54} | ω_{T70} |
| 1 | 1 | 1 |
| 2 | 1 | 3 |
| 3 | 1 | 4 |
| 4 | 1 | 5 |
| 5 | 2 | 2 |
| 6 | 2 | 3 |
| 7 | 2 | 4 |
| 8 | 2 | 5 |
| 9 | 3 | 4 |

### Example 3

Background: The adoption of CGM results in vast amounts of data, but their interpretation is still more art than exact science. The International Consensus on Time in Range (TIR) proposed the widely accepted TIR system of metrics, which we now take forward by introducing a finite and fixed set of clinically-similar clusters (CSCs), such that within a cluster the TIR metrics of the daily CGM profiles are homogeneous.

Methods: CSC definition and validation used 204,710 daily CGM profiles in health, type 1 and type 2 diabetes (T1D, T2D), on different treatments. The CSCs were defined using 23,916 daily CGM profiles (Training data), and the final fixed set of CSCs was obtained using another 37,758 profiles (Validation data). Testing data (143,036 profiles) was used to establish the robustness and generalizability of the CSCs.

Results: The final set of CSCs contains 35 clusters. Any daily CGM profile was classifiable to a single CSC which faithfully approximated common glycemic metrics of the daily CGM profile, as evidenced by regression analyses with 0 intercept (R-squares >0.81, e.g., correlation >0.9, for all TIR and most other metrics. The CSCs distinguished CGM profiles in health, T2D, and T1D on different treatments, and allowed tracking of the daily changes in a person's glycemic control over time.

Conclusion: Any daily CGM profile can be classified into one of [only]35 prefixed CSCs, which enables a host of applications, e.g., tabulated data interpretation and algorithmic approaches to treatment, CGM replacement for clinical tests, database indexing, pattern recognition, and tracking disease progression.

### Introduction

The widespread adoption of continuous glucose monitoring (CGM) technologies inevitably creates vast amounts of data; for example, two recent reports of real-life use of an artificial pancreas system were based on over 1.5 billion data points. Over the years, a number of glycemic control metrics were introduced, with the general objective of aggregating CGM data to convey a meaningful clinical message. Some existing measures based on self-monitoring data, such as MAGE (Mean amplitude of glucose excursions) and LBGI/HBGI (Low and High BG Indices) have been adapted for CGM use as well: the adaptation of MAGE for CGM data followed the classic time-independent structure of this measure, and therefore in this case CGM was only used as a source for amplitude assessment; the adaptation of the LBGI and the HBGI accounted for differences between SMBG and CGM data. The Mean of Daily Differences (MODD) was introduced as a measure of intra-day variability, and the Continuous Overlapping Net Glycemic Action (CONGA) was presented as a composite index of the magnitude and the timing of blood glucose (BG) fluctuations captured over various time periods. The standard deviation of the BG rate of change was used as a marker of the stability of the metabolic system over time, based on the premise that more erratic BG changes are signs of system instability. An array of standard deviations was introduced to reflect glucose variability contained within different clinically-relevant periods of CGM data, and the clinical interpretation of various CGM-based metrics of glucose variability was discussed 8 An early review of the statistical methods available for the analysis of CGM data included several graphs, such as Poincard plot of system stability, and the Variability-Grid Analysis (VGA) used to visualize glycemic fluctuations captured by CGM and the efficacy of Automated Insulin Delivery (AID). Perspectives published in Reviews on Biomedical Engineering11 and Diabetes Care12 evaluated the methods for computing and visualization of glucose variability in the context of its relationship to risk for hypoglycemia. The Glucose Management Indicator (GMI) was introduced as a CGM-based approximation of HbA1c assay. The latest addition to the family of glucose control metrics was the Glycemia Risk Index (GRI), which was based on the collective opinions of a number of physicians, and attempted to balance the risks for hypo and hyperglycemia in a single metric.

Consequently, the CGM field became overloaded not only by voluminous data sets, but also by a multitude of metrics used to assess various aspects of glycemic control. For reference, many, but not all, of the existing metrics were discussed in detail in a 2017 paper published in Nature Reviews Endocrinology. The general thesis was that CGM-based metrics should typically include some notion of the timing of CGM readings, not only of their amplitude. This is because CGM data represent time series of equally spaced in time glucose observations—a property that enables analytics way beyond the reach of the traditional MAGE, LBGI/HBGI, MODD, CONGA, GRI, or any other amplitude-based metric entertained over the years. For example, contemporary algorithms enabling AID are possible only because of the temporal information carried by the CGM data stream.

Most recently, tidying up the clutter of multiple glycemic markers, it was shown that virtually all glycemic control metrics introduced over the years, are described by [only] two “essential metrics”—exposure to hyperglycemia and risk for hypoglycemia, meaning that the quantitative representation of glycemic control is a rather simple 2-dimensional structure. In 2019, the International Consensus on Time in Range (TIR, typically 70-180 mg/dL), proposed TIR as a primary CGM-based metric of glycemic control and set clinical targets for its use. Given that time below range (TBR), TIR, and time above range (TAR) always add up to 100 percent, the TIR system of metrics is a good representation of the 2-dimensional structure of glycemic control—TBR as a metric of risk for hypoglycemia and TAR (or, equivalently, TIR) measuring exposure to hyperglycemia. This system is based on the Ambulatory Glucose Profile (AGP), introduced as a template for data presentation and visualization originally developed by Mazze et. al for self-monitoring data. Based on the AGP, the standardized CGM report now incorporates core CGM metrics and targets along with a 14-day composite glucose profile as an integral component of clinical decision making. This format was endorsed by the international consensus on TIR17 and was also referenced by the American Diabetes Association 2019 Standards of Care20 and the AACE consensus on use of CGM.21 The AGP report is now adopted by many CGM device manufacturers in their CGM companion software, and is proposed by international consensuses as a standardized output in the evaluation of AID technologies, and the presentation of results from clinical trials.

The widely adopted TIR system of metrics defines 5 times in ranges for CGM glucose values. These times in ranges are used to provide a numerical interpretation of the AGP: Level 2 hypoglycemia—below 54 mg/dl, Level 1 hypoglycemia—from 54 to 69 mg/dL, within Target Range (TIR)—70 to 180 mg/dL, Level 1 hyperglycemia—from 180 to 250 mg/dL, and Level 2 hyperglycemia—above 250 mg/dL.

These boundaries may vary according to the Consensus recommendations for different types of diabetes, but the concept remains the same. A static visual and quantitative depiction—a snapshot of (typically 14 days) of CGM data—is therefore well established by the AGP/TIR representation. However, the TIR system of metrics does not reflect the inter-day variability of the CGM data (except in terms of the AGP cloud), or the progression (improvement/deterioration), of glycemic control overtime. Given that the main advantage of CGM is measuring time series of glucose values and capturing the process of glycemic control as it evolves, equipping the TIR system with a temporal component becomes essential. Thus, this manuscript takes the next step of advancing the AGP/TIR concept, by establishing a fixed and finite set of clinically similar clusters (CSCs), which faithfully represent the multitude of all daily CGM profiles with relatively few (N=35) fixed CSCs, and allow tracking of daily glycemic changes over time in a table-lookup format.

### Materials and Methods

### Data:

Sixteen deidentified archival data sets were used in this work, as detailed in Table 7, which includes demographic information and summary statistics for the study participants, type of diabetes (T1D, T2D), or health, and treatment modality, e.g., multiple daily insulin injections (MDI), continuous subcutaneous insulin delivery via insulin pump (CSII), or automated insulin delivery (AID).

TABLE 7
Characteristics of the participants in the 16 studies used in this work.

| | | # | | | | | Study | No. Daily |
|---|---|---|---|---|---|---|---|---|
| | Health | Participants | Age, | BMI, | HbA1c, | Treatment | Duration, | CGM |
| Study Name | State | (% Female) | years | kg/m² | % | Modality | weeks | Profiles |
| CITY ERROR! | T1D | 153 (49.7) | 17.5 (2.9) | 25.4 (5.0) | 9.1 (1.0) | MDI, CSII | 26 | 20,226 |
| REFERENCE SOURCE | | | | | | | | |
| NOT FOUND. | | | | | | | | |
| D_{CLP}1 ERROR! | T1D | 125 (47.2) | 32.5 (14.9) | 26.7 (5.2) | 7.5 (0.9) | AID, CSII | 13 | 5,459 |
| REFERENCE SOURCE | | | | | | | | |
| NOT FOUND. | | | | | | | | |
| D_{CLP}3 ERROR! | T1D | 168 (50.0) | 32.8 (16.0) | 25.8 (5.3) | 7.6 (1.0) | AID, CSII | 26 | 24,450 |
| REFERENCE SOURCE | | | | | | | | |
| NOT FOUND. | | | | | | | | |
| D_{CLP}5 ERROR! | T1D | 93 (50.5) | 10.7 (2.0) | 19.4 (4.0) | 7.7 (1.1) | AID, CSII | 16 | 10,050 |
| REFERENCE SOURCE | | | | | | | | |
| NOT FOUND. | | | | | | | | |
| DIAMOND1 ERROR! | T1D | 158 (44.3) | ≥25 | — | — | MDI | 24 | 12,699 |
| REFERENCE | | | | | | | | |
| SOURCE NOT FOUND. | | | | | | | | |
| DIAMOND2 ERROR! | T2D | 158 (56.3) | ≥25 | — | — | MDI | 24 | 7,280 |
| REFERENCE | | | | | | | | |
| SOURCE NOT FOUND. | | | | | | | | |
| DSS1 ERROR! | T1D | 83 (55.4) | 35.1 (14.9) | 27.2 (6.0) | 7.5 (1.1) | MDI | 12 | 4,494 |
| REFERENCE SOURCE | | | | | | | | |
| NOT FOUND. | | | | | | | | |
| MDEX ERROR! | T1D | 15 (40.0) | 32.3 (9.3) | 24.7 (2.8) | 7.1 (0.7) | CSII | 6 | 145 |
| REFERENCE SOURCE | | | | | | | | |
| NOT FOUND. | | | | | | | | |
| NDIAB ERROR! | Healthy | 163 (68.1) | 31.4 (21.2) | 22.2 (4.2) | 5.2 (0.3) | — | 1.4 | 888 |
| REFERENCE SOURCE | | | | | | | | |
| NOT FOUND. | | | | | | | | |
| NIGHTLIGHT | T1D | 81 (66.3) | 42.3 (11.9) | 29.4 (5.7) | 7.4 (1.0) | AID, CSII | 32 | 12,329 |
| ERROR! REFERENCE | | | | | | | | |
| SOURCE NOT FOUND. | | | | | | | | |
| REPLACE-BG | T1D | 226 (49.6) | 44.0 (13.8) | 27.3 (4.4) | 7.3 (0.7) | CSII | 26 | 36,918 |
| ERROR! REFERENCE | | | | | | | | |
| SOURCE NOT FOUND. | | | | | | | | |
| RT-CGM ERROR! | T1D | 444 (55.0) | 25.1 (15.8) | 23.5 (4.4) | 7.4 (0.9) | MDI, CSII | 26 | 27,938 |
| REFERENCE SOURCE | | | | | | | | |
| NOT FOUND. | | | | | | | | |
| SENCE ERROR! | T1D | 144 (50.0) | 5.2 (1.7) | 17.2 (3.0) | 8.3 (0.7) | MDI, CSII | 26 | 14,431 |
| REFERENCE SOURCE | | | | | | | | |
| NOT FOUND. | | | | | | | | |
| SEVHYPO ERROR! | T1D | 187 (46.5) | ≥60 | 27.1 (4.7) | 7.7 (1.2) | MDI, CSII | 2 | 1,170 |
| REFERENCE SOURCE | | | | | | | | |
| NOT FOUND. | | | | | | | | |
| UVA-TRIALNET | Healthy | 58 (56.9) | 24.2 (10.9) | 23.9 (5.7) | 5.3 (0.3) | — | 1 | 281 |
| ERROR! REFERENCE | | | | | | | | |
| SOURCE NOT FOUND. | | | | | | | | |
| WISDM ERROR! | T1D | 206 (51.7) | 68.0 (5.7) | 27.1 (4.6) | 7.5 (0.9) | MDI, CSII | 26 | 26,152 |
| REFERENCE SOURCE | | | | | | | | |
| NOT FOUND. | | | | | | | | |
| Total | | 2,462 (52.6) | | | | | | 204,710 |

Statistics are presented as mean (SD) unless otherwise indicated. T1D denotes type 1 diabetes, T2D type 2 diabetes, BMI body mass index, CGM continuous glucose monitoring, MDI multiple daily injections, CSII—continuous subcutaneous insulin delivery via insulin pump, AID—automated insulin delivery.

These data were collected in clinical trials done at the University of Virginia's Center for Diabetes Technology, or available at the public data repository of the Jaeb Center for Health Research, Tampa, Florida. The references to these studies are as follows: CITY,24 DCLP1,25 DCLP3,26 DCLP5,27 DIAMOND1,28 DIAMOND2,29 DSS1,30 MDEX,31 NDIAB,32 NIGHTLIGHT,33 REPLACE-BG,34 RTCGM, 35 SENCE,36 ASEVHYPO,37 UVA-TRIALNET,38 and WISDM.39 In total, these data sets contained CGM traces for 2,462 individuals (52.6 percent female) and 204,710 daily CGM profiles, or approximately 560 years of data in health, T1D, and T2D. Most (95.9%) of the daily CGM profiles were generated by people with T1D treated by MDI, CSII, or AID. Two studies focused on children with T1D (DCLP5 and SENCE). The DIAMOND2 study had participants with T2D on MDI treatment and represented 3.6% of the daily CGM profiles. The NDIAB and UVA-TRIALNET studies contained data from people without diabetes. Glycemic control, assessed by mean HbA1c at baseline, ranged between 5.2% for those without diabetes (NDIAB study) to 9.1% (CITY study). In health, people generally had less than 7 days of data, while the participants in the vast majority of diabetes studies had 5 or more weeks of data on average.

Data Preprocessing and Separation into Training, Validation, and Testing Data

A previously published procedure was used to process the CGM time series and define the daily CGM profiles, where a daily CGM profile is a time series of 288 blood glucose data points collected every 5 minutes during the midnight-to-midnight (24-hour) period—see Section III, Lobo et al. The daily CGM profiles from these 16 different studies formed 3 different data sets, each with a distinct purpose:
1. The Training data consisted of 23,916 daily CGM profiles sampled from the DCLP1, DCLP3, DIAMOND1, DIAMOND2, DSS1, and NIGHTLIGHT studies and was used to define the candidate sets of CSCs.
2. The Validation data consisted of 37,758 daily CGM profiles sampled from the same 6 studies as the Training data and was used to assess the performance of candidate sets of CSCs, and then select and fix the final set of CSCs.
3. The Testing data consisted of 143,036 daily CGM profiles taken from the CITY, DCLP5, DIAMOND2, NDIAB, MDEX, REPLACE-BG, RT-CGM, SENCE, SEVHYPO, UVA-TRIALNET and WISDM studies, and was used to evaluate the robustness and generalizability of the final selected set of CSCs.

These data sets were constructed such that there is no intersection between the Training, Validation, and Testing data. Moreover, the Testing data was derived from studies which used different methodologies and different generations of CGM technology to those studies used in the Training and Validation data.

### Analytics: Establishing Afinal Set of CSCs and Daily Profile Classification

Step 1: A set of CSCs is defined and then fixed, with the property that for any daily CGM profile there is a CSC that approximates the 5 standard times in ranges of said daily CGM profile, abbreviated here as follows: T54 (percent of CGM time below 54 mg/dl); T70 (percent of CGM time below 70 mg/dl), TIR (percent of CGM time within 70-180 mg/dl), T180 (percent of CGM time above 180 mg/dl), and T250 (percent of CGM time above 250 mg/dl). Such an approximation guarantees that key clinically-relevant characteristics of the daily CGM profile are preserved. The CSC set is defined using hierarchical clustering, where weighting of the inputs is varied until the set of CSCs have the desired performance approximating the vector {T54, T70, TIR, T180, T250}.

Step 2: A procedure is developed for mapping a daily CGM profile to its closest CSC, which involves computing a similarity metric (e.g., Euclidean distance) between the candidate daily CGM profile and the centroids of each CSC, and then selecting the CSC with the best similarity metric value. The similarity metric is computed in the “space” defined by all possible vectors {T54, T70, TIR, T180, T250}. When these two steps are accomplished, any daily CGM profile can be mapped to a CSC that approximates the five times in ranges of the original daily CGM profile, and the sequence of CSCs for an individual can be used as a surrogate for the progression of glycemic control of this individual.

Remembering that the glycemic control space is essentially two-dimensional,16 approximating a daily CGM profile with a CSC, i.e., minimizing the distance between the two in terms of {T54, T70, TIR, T180, T250}, guarantees that any other metric of glycemic control derived from said daily CGM profile will be approximated by the CSC as well. In addition to metric approximation, the sequence of CSCs provides information about the timing and the inter-day variability of the clinically-relevant glycemic events of a patient.

An expanded mathematical description of the procedure described in this section is provided in the Supplementary Material.

### Result

### Final Set of CSCs: Review and Interpretation

The procedure described in the previous section resulted in a final set of 35 clinically-similar clusters. The results in this section use the Testing data and serve as an external validation of the CSC method, and as an illustration of its potential for clinical applications. The CSC Index indicates the degree of glycemic control, as represented by TIR alone. TIR is highest in the CSCs with lower indices, e.g., 1,2,3 and lowest in the CSCs with highest indices, e.g., 33, 34, 35. For example, CSC 1 has TIR=85.4% while CSC 35 has TIR=2.4%. However, CSCs with adjacent indices, while having similar TIRs, can be very different in terms of exposure to hypo or hyperglycemia. For example, in CSC 12, TIR=46.4% and the rest is distributed between Levels 1 and 2 hypoglycemia, with significant presence of readings below 54 mg/dl (T54=27.3%). In contrast, for CSC 13, TIR=44.9% but the rest is accounted for by hyperglycemia (T180=30.4% and T250=21.9%).

Table 8 lists all CSCs with their respective values of {T54, T70, TIR, T180, T250}, i.e., the centroids defining each CSC, and the number/percentage of daily CGM profiles associated with each CSC in the Testing data.

TABLE 8
A list of all CSCs with their respective values
of {T54, T70, TIR, T180, T250},

| CSC | T54 | T70 | TIR | T180 | T250 | Occurrence in Test Data Set | |
|---|---|---|---|---|---|---|---|
| Index | % | % | % | % | % | Count | % |
| 1 | 0.4 | 2.1 | 85.4 | 10.8 | 1.2 | 41,353 | 28.9 |
| 2 | 2.6 | 15.3 | 76.5 | 4.8 | 0.9 | 4,199 | 2.9 |
| 3 | 2.6 | 22.8 | 72.7 | 1.8 | 0.1 | 476 | 0.3 |
| 4 | 18.5 | 9.6 | 71.4 | 0.4 | 0.0 | 431 | 0.3 |
| 5 | 0.2 | 0.8 | 63.9 | 27.4 | 7.7 | 32,328 | 22.6 |
| 6 | 2.7 | 7.5 | 59.7 | 23.9 | 6.2 | 6,770 | 4.7 |
| 7 | 6.7 | 19.5 | 57.8 | 11.9 | 4.0 | 1,115 | 0.8 |
| 8 | 27.6 | 5.9 | 54.4 | 12.1 | 0.0 | 187 | 0.1 |
| 9 | 2.1 | 35.8 | 48.6 | 9.0 | 4.5 | 59 | 0.0 |
| 10 | 20.8 | 31.2 | 47.9 | 0.0 | 0.0 | 54 | 0.0 |
| 11 | 4.0 | 14.3 | 46.5 | 23.6 | 11.6 | 1,294 | 0.9 |
| 12 | 27.3 | 15.1 | 46.4 | 10.2 | 1.0 | 104 | 0.1 |
| 13 | 0.9 | 1.9 | 44.9 | 30.4 | 21.9 | 14,140 | 9.9 |
| 14 | 17.0 | 2.4 | 44.8 | 34.0 | 1.7 | 283 | 0.2 |
| 15 | 6.3 | 27.3 | 44.7 | 14.9 | 6.9 | 127 | 0.1 |
| 16 | 0.1 | 0.4 | 41.5 | 50.7 | 7.3 | 9,787 | 6.8 |
| 17 | 21.3 | 8.6 | 39.4 | 21.5 | 9.2 | 195 | 0.1 |
| 18 | 19.4 | 3.3 | 38.9 | 13.9 | 24.5 | 181 | 0.1 |
| 19 | 0.5 | 1.1 | 36.7 | 22.0 | 39.6 | 7,946 | 5.6 |
| 20 | 2.2 | 7.2 | 36.4 | 52.5 | 1.7 | 1,172 | 0.8 |
| 21 | 6.6 | 9.8 | 36.3 | 12.3 | 35.0 | 1,246 | 0.9 |
| 22 | 41.0 | 11.5 | 34.2 | 11.5 | 1.9 | 46 | 0.0 |
| 23 | 9.5 | 18.9 | 28.9 | 13.8 | 28.9 | 93 | 0.1 |
| 24 | 0.1 | 0.5 | 28.2 | 46.2 | 25.0 | 6,466 | 4.5 |
| 25 | 9.8 | 9.9 | 27.7 | 30.1 | 22.6 | 354 | 0.2 |
| 26 | 11.5 | 17.0 | 26.4 | 33.7 | 11.5 | 84 | 0.1 |
| 27 | 5.8 | 6.7 | 19.3 | 53.4 | 14.8 | 767 | 0.5 |
| 28 | 0.0 | 20.1 | 14.9 | 35.4 | 29.5 | 37 | 0.0 |
| 29 | 1.9 | 8.7 | 14.9 | 14.1 | 60.4 | 733 | 0.5 |
| 30 | 0.1 | 0.3 | 14.4 | 19.8 | 65.3 | 3,231 | 2.3 |
| 31 | 0.1 | 0.1 | 13.2 | 63.2 | 23.4 | 1,857 | 1.3 |
| 32 | 20.8 | 2.7 | 13.1 | 20.0 | 43.4 | 83 | 0.1 |
| 33 | 0.0 | 0.0 | 12.5 | 82.7 | 4.7 | 466 | 0.3 |
| 34 | 0.1 | 0.2 | 10.5 | 38.4 | 50.8 | 4,226 | 3.0 |
| 35 | 0.0 | 0.0 | 2.4 | 8.9 | 88.6 | 1,146 | 0.8 |

### Establishing a Final Set of CSCs and Daily Profile Classification

The times in ranges {T54,1T70, TIR, T180, T250} were used as the input for a single daily CGM profile when performing hierarchical clustering. Inputs were generated using all daily CGM profiles in the Training data. The scipy.cluster.hierarchy Python module implementation of hierarchical clustering with the centroid algorithm17 was used to calculate Euclidean distances between two rows of input. Because we wanted to ensure that the time below range behavior was faithfully captured by the CSCs we weighted the T54 and 170 input columns greater than the TIR, T180 and T250 input columns. Each CSC is a collection of daily CGM profiles such that each daily CGM profile in the collection has essentially the same time in ranges. For a given set of inputs (determined using the daily CGM profiles in the Training data and the weights chosen for the Very Low and Low columns), the hierarchical clustering algorithm produced a dendrogram indicating the hierarchical relationships between the daily CGM profiles in the Training data.

“Cutting” the dendrogram at a specific height produced a clustering with a specific number of clusters. The evaluation of each candidate set of CSCs used the Validation data. The centroids of the CSCs were used to classify each daily CGM profile in the Validation data. Let dpᵢⱼₖ be the time in range k for the i-th daily CGM profile from individual i, CSCₖ(dpᵢⱼ) be the time in range k for the CSC that the j-th daily CGM profile from individual i is classified as, and where kϵ{T54, T70, TIR, T180, T250}.

Furthermore, let

$${dp}_{ik} = \frac{1}{\vert {J}_{i} \vert} {\sum }_{j ∈ {J}_{i}} {dp}_{ijk}$$

$$and let$$

$$C {SC}_{k} ( {dp}_{i} ) = \frac{1}{\vert {J}_{i} \vert} {\sum }_{j ∈ {J}_{i}} C {SC}_{k} ( {dp}_{ij} )$$

where j is the set of all daily CGM profiles of individual i. A single “optimal” set of CSCs:

Maximized the r-squared value of the linear regression through the set of points (dpⱼₖ, CSCₖ(dpᵢ) for each k, and ensured that the absolute value of the relative effect size was less than 0.15 for each k, where the relative effect size is computed as the mean of the average differences between (dpᵢⱼₖ, and CSCₖ(dpᵢⱼₖ) divided by the standard deviation across all dpᵢⱼₖ.

We explored 9 different weightings of the Very Low and Low input columns (i.e., 9 different sets of input to the hierarchical clustering algorithm), which resulted in 166 candidate sets of CSCs being evaluated. The final fixed set of CSCs had 35 clusters. Each CSC is defined by a centroid. The centroid for a given CSC is calculated using the daily CGM profiles in the Training data which were assigned to the CSC.

It is evident that “extreme hypoglycemia” CSCs, such as #10 and #22 are seen infrequently (54 and 46 times out of 143,036 daily CGM profiles respectively), while “moderate” clusters, such as #6 and #11 (6,770 and 1,294 times, respectively)), or clusters primarily associated with hyperglycemia, e.g., #34 and #35 (4,226 and 1,146 times respectively), are more frequent, which is to be expected, given that the majority of daily CGM profiles in this analysis come from individuals with T1D on various treatments.

The most frequent CSCs are #1 and #5 by a large margin (Table 8), which is a function of including people in health and on advanced treatments, such as T1D on AID, or T2D on CGM.

### Tracking the Progression of Glycemic Control Over Time

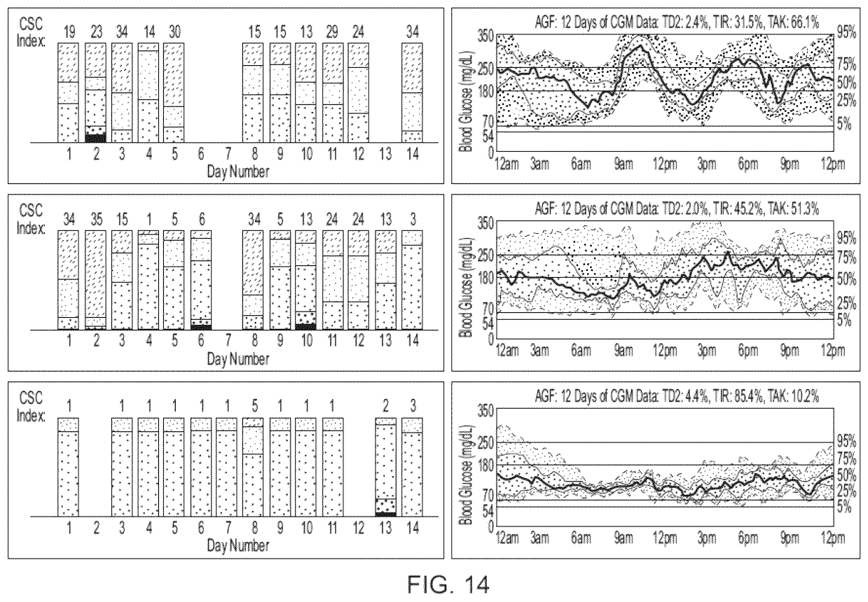

Over time, the sequence of daily CGM profiles generated by each person can be presented as a sequence of CSCs which represents the progression of glycemic control of this individual over time. For example, a person in good glycemic control would visit fewer unique CSCs, generally with lower indices (indicating more time spent in range), while a person with volatile glucose variations would visit many more unique CSCs, where those CSCs visited would often have higher indices (indicating less time spent in target range and more time spent above or below range). FIG. 14 is a 3-panel plot which illustrates the progression of three individuals with T1D over 14 days. Panel A (the top row) presents data from a 6 year old boy with baseline HbA1c of 7.8% treated with MDI from the SENCE study, Panel B (the middle row) presents data from a 7 year old boy with baseline HbA1c of 7.9% on CGM+CSII from the DCLP5 study,27 while Panel C (the bottom row) presents data from a 9 year old boy with baseline HbA1c of 7.8% on AID from the DCLP5 study, to represent these three treatment modalities. These individuals had the same gender, similar ages, and essentially the same baseline HbA1c, but thereafter their trajectories digress and the daily transitions between CSCs and the number/index of CSCs visited differ substantially.

Further, FIG. 14 includes the AGP for each of the presented 14-day CSC traces. It is evident that the sequences of CSCs faithfully represent the information carried by the AGP, and adds information about the individual's daily changes in glycemic control, their worst or best days, or any trends in treatment progress that may be occurring during the 14 days of observation. Moreover, it becomes evident that even a person on AID with a very stable glucose control who had TIR=85.4% during the observation period (Panel C), had one day with substantial hyperglycemia (day 8 classified in CSC #5), one hypoglycemic day (day 13, classified in CSC #2), and two days of incomplete data (days 2 and 12) when the AID system was not worn or malfunctioned—information that is not conveyed by the AGP, or the TIR system of metrics.

### CSC Visits as Representation of State of Health and Treatment Efficacy

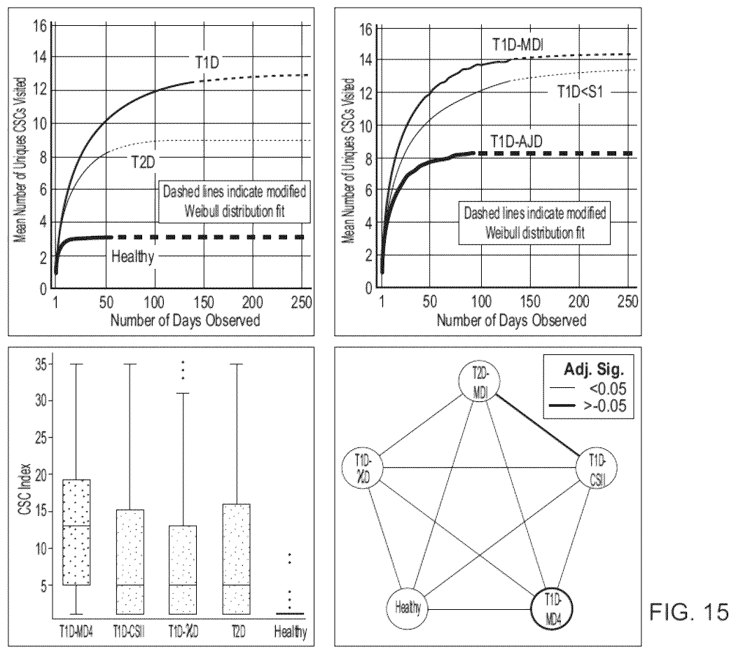

FIG. 15 is a 4-panel plot that illustrates the ability of the set of CSCs to distinguish between states of health and treatment modalities. One panel presents the average number of unique CSCs visited by people with T1D, T2D, and in health (solid lines). It is evident that in T1D the number of unique CSCs visited over time (e.g., 6 months) is greatest, while in health only 3 unique CSCs are visited on average. Another panel presents the same trajectories as in the first panel, but in this case differentiates between the treatments of individuals with T1D, namely, MDI, CSII, and AID. The dotted lines in these panels are Weibull distribution functions which have been fit to the data, and these fitted curves approximate the real trajectories very well. The Weibull fits have a certain probabilistic meaning which is beyond the scope of this manuscript. Another panel shows a Box plot of the CSC Index by state of health with T1D broken out by treatment modality. The plot confirms that on average the highest CSC indices are reached by people with T1D on MDI treatment, among those with diabetes AID the CSC Index lowest, and people in health visit only a few CSCs with low indices (all below 10). Finally, another panel presents a statistical pentagram with Bonferroni corrected pairwise comparisons between all 5 conditions under consideration. As seen, all pairwise differences are statistically significant, except for the difference between T1D on CSII and T2D.

### Relationship Between CSC, AGP, and Established Metrics of Glycemic Control

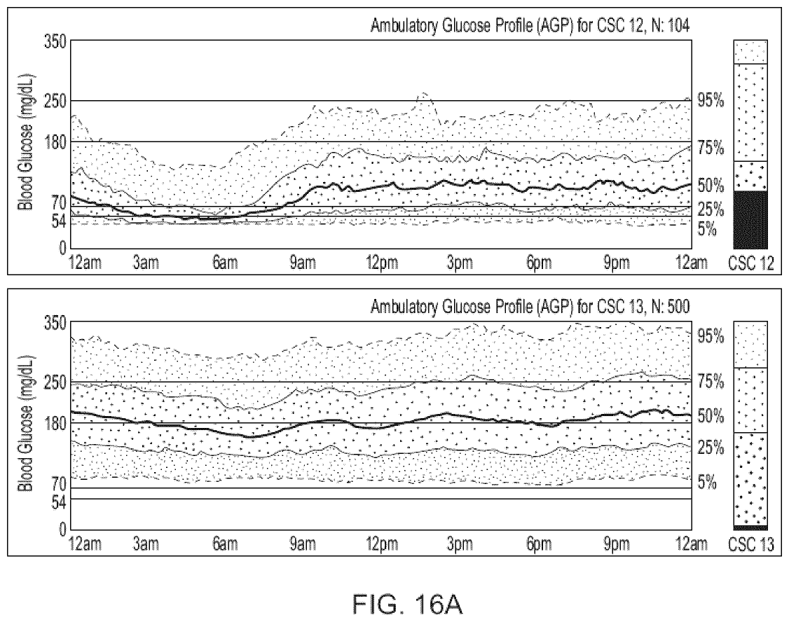

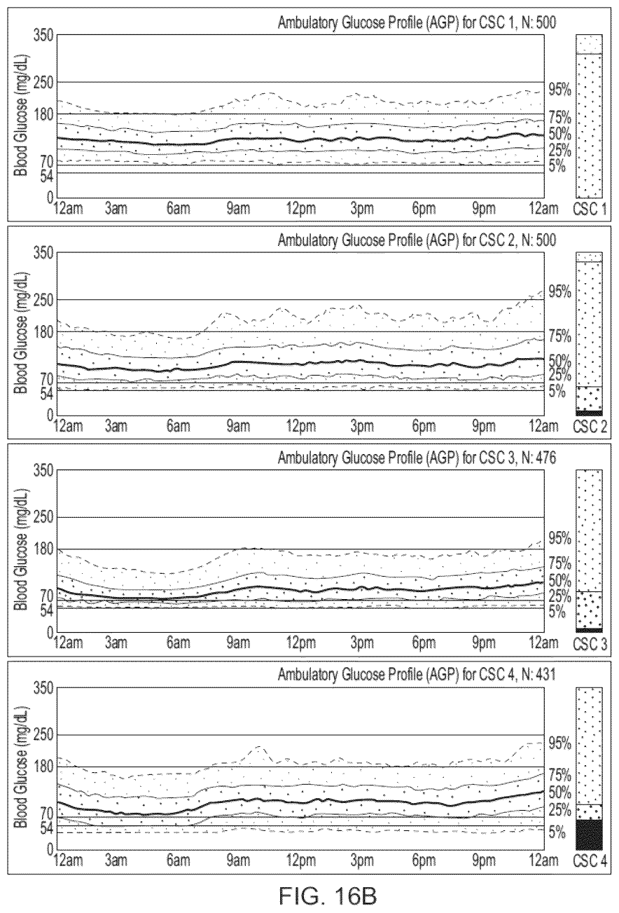

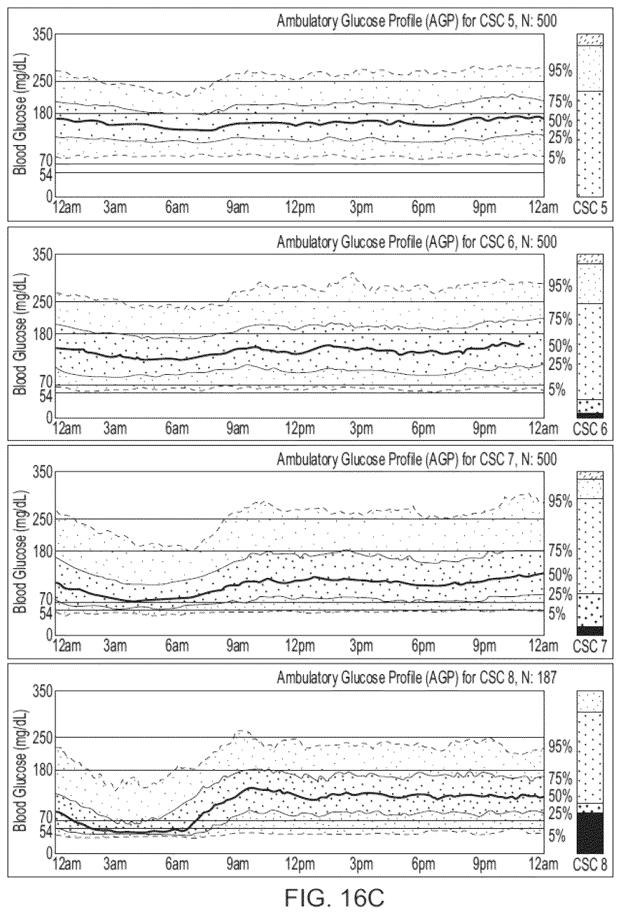

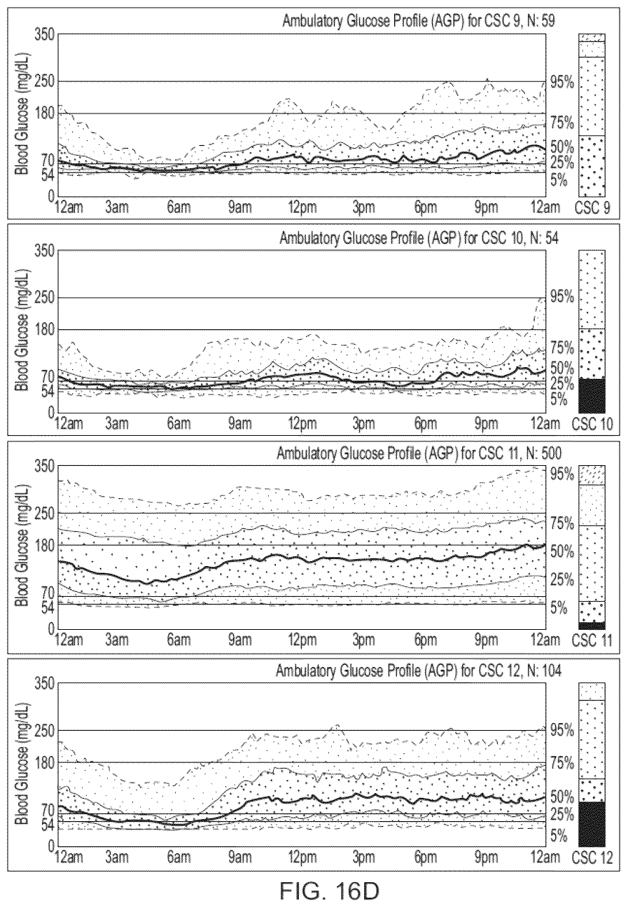

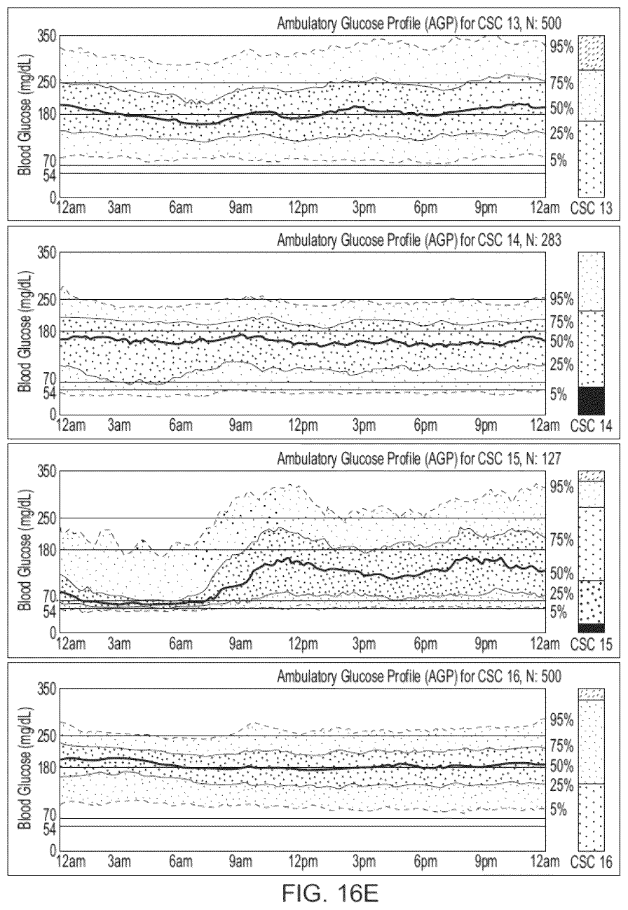

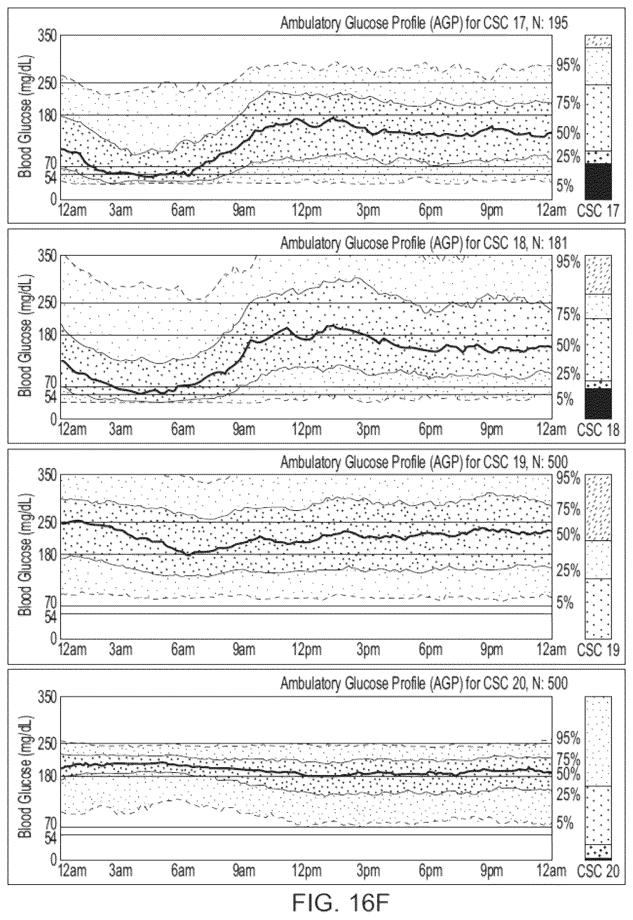

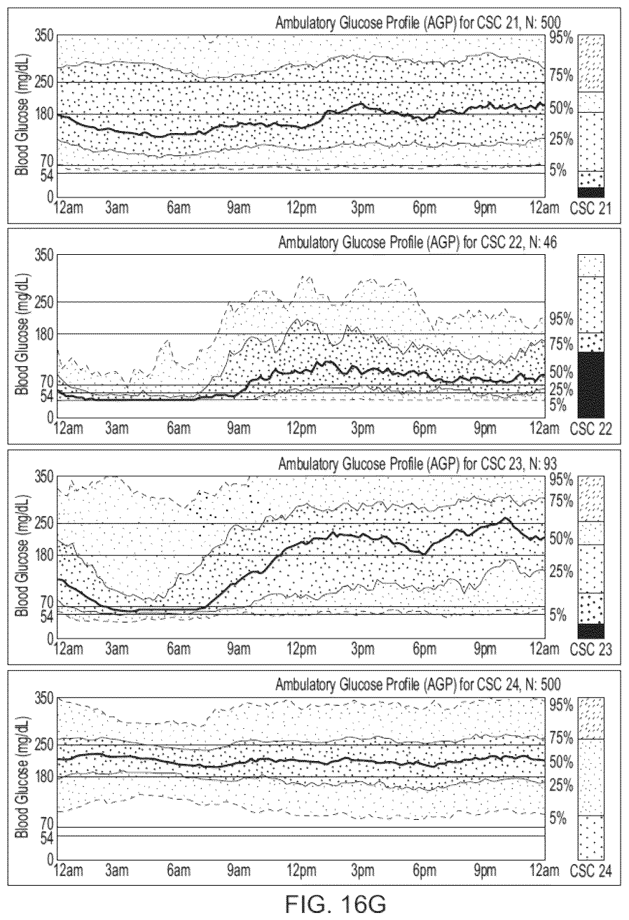

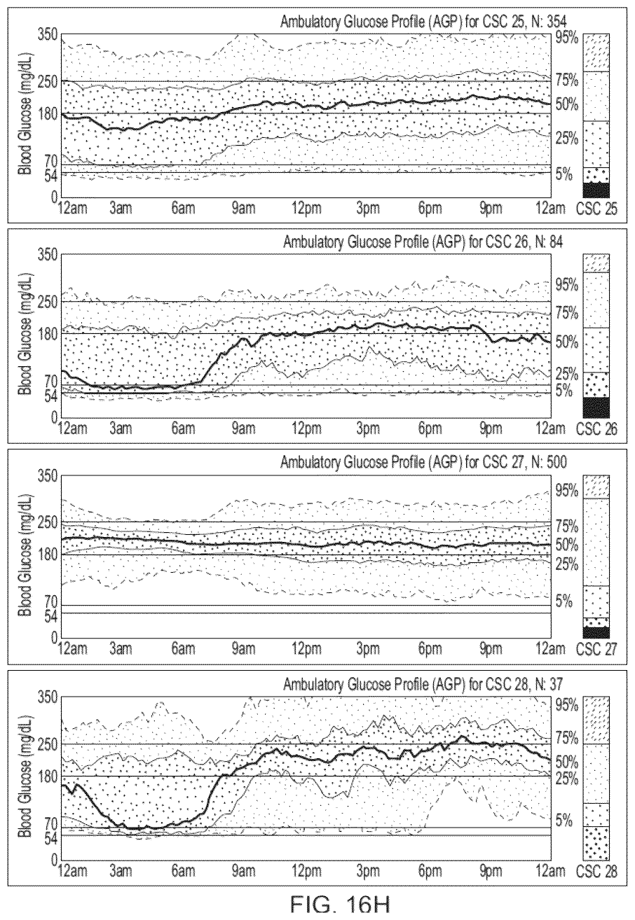

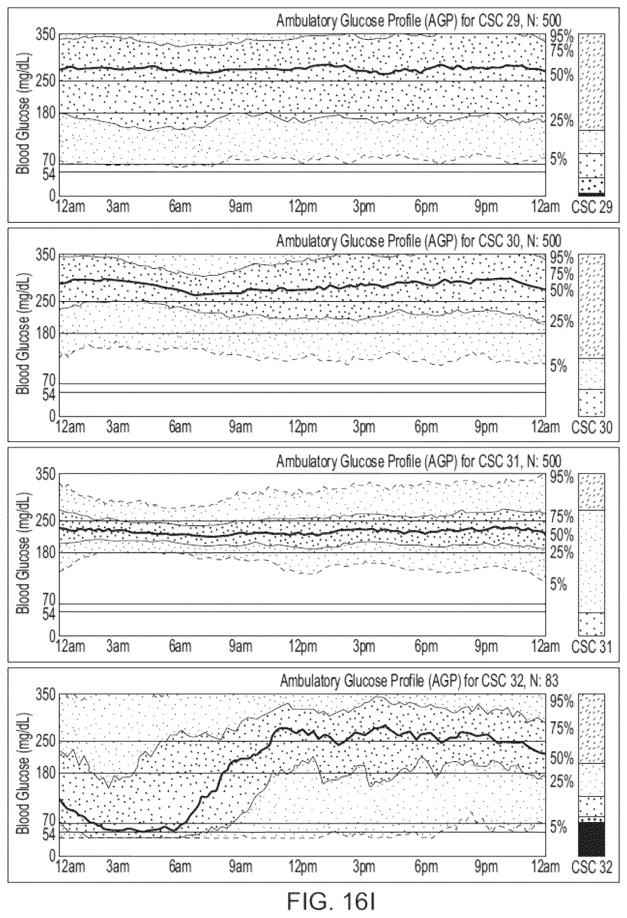

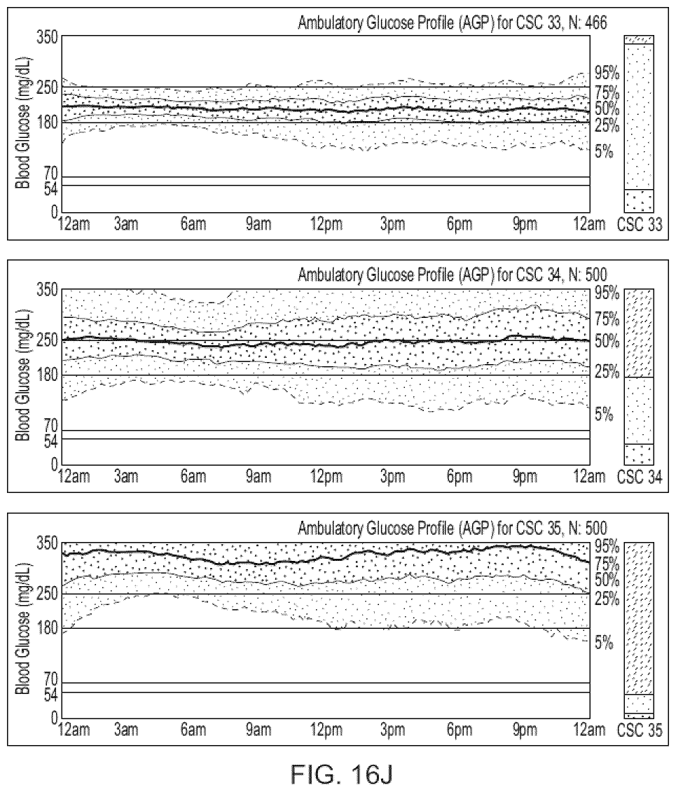

Each CSC represents a number of daily CGM profiles from different individuals. The relationship between the information carried by CSC and AGP is illustrated in FIG. 16A, which show the two aforementioned adjacent clusters, #12 and #13. For each of these CSCs we plot a version of the AGP which, instead of aggregating consecutive daily CGM profiles for an individual, in this case aggregates all daily CGM profiles associated with each CSC. As expected, the AGP associated with CSC #13 is shifted up, if compared to the AGP associated with CSC #12, while the AGP “clouds” are visually similar. This is to be expected, given that CSC #12 and #13 have similar TIRs (46.4% and 44.9% respectively), but differ substantially in terms of hypoglycemia and hyperglycemia. FIGS. 166-16J are plots similar to those shown in FIG. 16A for all 35 CSCs reported in this manuscript. Contrasting the lowest vs highest CSC indices (e.g. #1, #2 vs #34, #35) clearly shows the effect of good glycemic control vs profiles associated primarily with hyperglycemia. Particularly instructive are CSCs such as #28 or #32, which indicate high volatility of glycemic control, with both substantial hypo- and hyperglycemia.

To confirm the proposition that CSCs represent faithfully the values of commonly accepted metrics of glycemic control of their member daily CGM profiles, Table 9 presents univariate regression analyses with zero intercept. In each regression, the dependent variable is a metric computed from the daily CGM profiles and the independent variable is the same metric computed from the CSC centroids associated with the daily CGM profile. As evidenced from Table 9, the slope of all regressions is close to 1, indicating that the values of the metrics computed from CSCs and from their associated daily CGM profiles lie close to the identity line. Further, the R-square values are high (generally above 0.81, which corresponds to correlations above 0.9), indicating that the metrics computed from CSCs account for a good portion of the variance carried by the original daily CGM profiles (with the exception of the Coefficient of Variation, for reasons addressed in the Discussion).

TABLE 9
Metrics of glycemic control computed from daily
CGM profiles and their respective CSCs

| | Linear | | |
|---|---|---|---|
| | Regression Results | | |
| | Inter- | | R- |
| | cept | Slope | Square |
| T54 | 0 | 1.068 | 0.876 |
| T70 | 0 | 0.940 | 0.915 |
| TIR | 0 | 1.038 | 0.974 |
| T180 | 0 | 0.984 | 0.898 |
| T250 | 0 | 1.004 | 0.974 |
| Mean Glucose | 0 | 0.986 | 0.903 |
| Coefficient of Variation | 0 | 0.983 | 0.579 |
| Glucose Management Indicator | 0 | 0.990 | 0.905 |
| (GMI)^{Error! Reference source not found.} | | | |
| Glycemic Risk Index (GRI) | 0 | 0.971 | 0.960 |
| Low BG Index (LBGI) | 0 | 1.074 | 0.831 |
| High BG Index (HBGI) | 0 | 1.008 | 0.958 |

### DISCUSSION

After years of fixation on a single metric of glycemic control, e.g. HbA1c, the medical community increasingly appreciates that contemporary technologies, such as CGM, provide the means for in-depth look at a person's state and dynamics of glucose fluctuations. The system of TIR metrics accepted by the International Consensus on TIR17 and its extensions to AID and clinical trial reporting, is an excellent step in this direction, as long as TIR alone does not become the next sole marker of treatment efficacy and attention is paid to other quantities, such as risk for hypoglycemia. Streamlining further the multitude of glycemic metrics, it was shown that two essential dimensions are sufficient to capture the information carried by virtually all metrics introduces to date: exposure to hyperglycemia, and risk for hypoglycemia.

The one exception is the Coefficient of Variation, which has been shown incompatible with the TIR system of metrics and is generally controversial, due to the fact that it is a ratio of two quantities (SD and mean), both of which are typically reduced by a successful treatment. The TIR system of metrics covers the two dimensions well, with certain mathematical redundancy because the TIR components add up to 100%, which is clinically OK, but may pose challenges to some statistical methods. This, we can consider that there is now an established approach to reviewing CGM data, which includes the AGP and its adjacent TIR metrics, and is sufficiently solid to allow a framework for classification and tracking of daily CGM profiles over time to be built upon it.

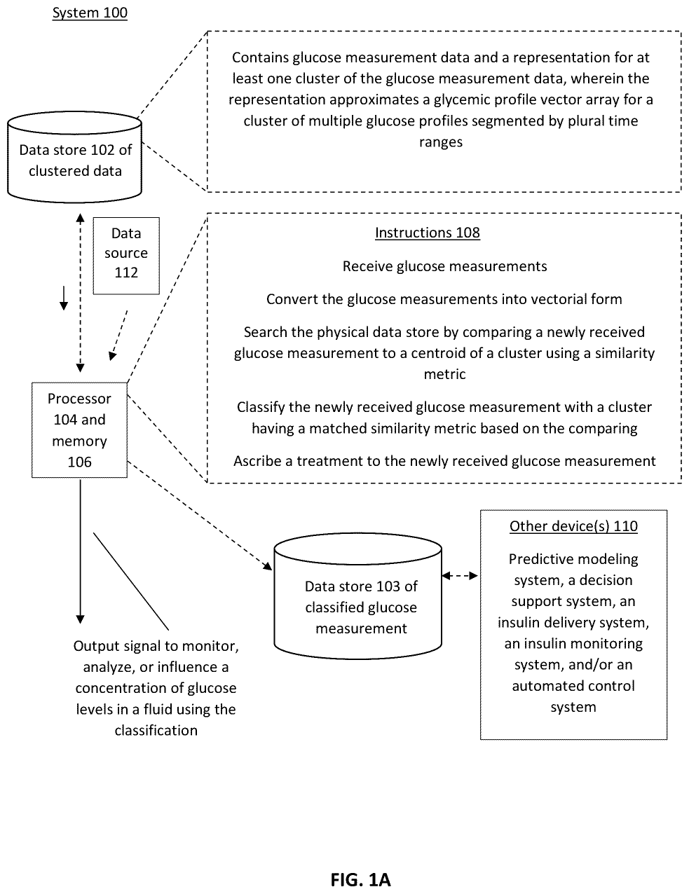

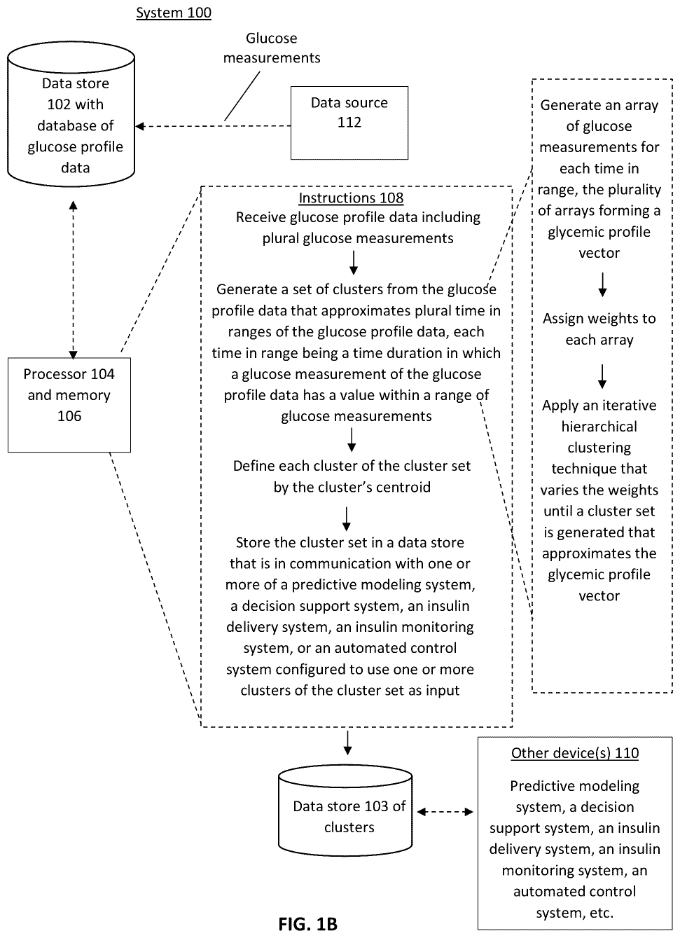

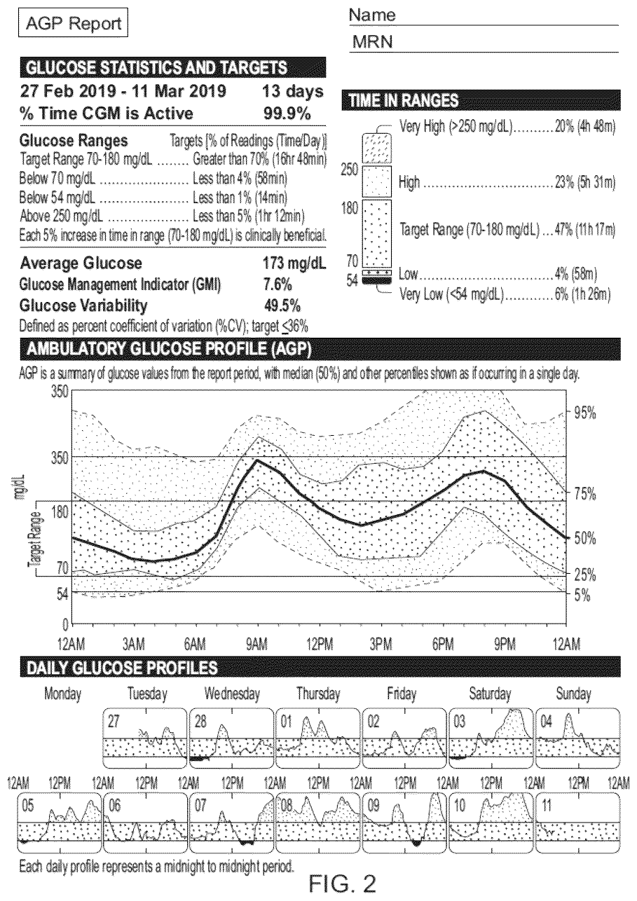

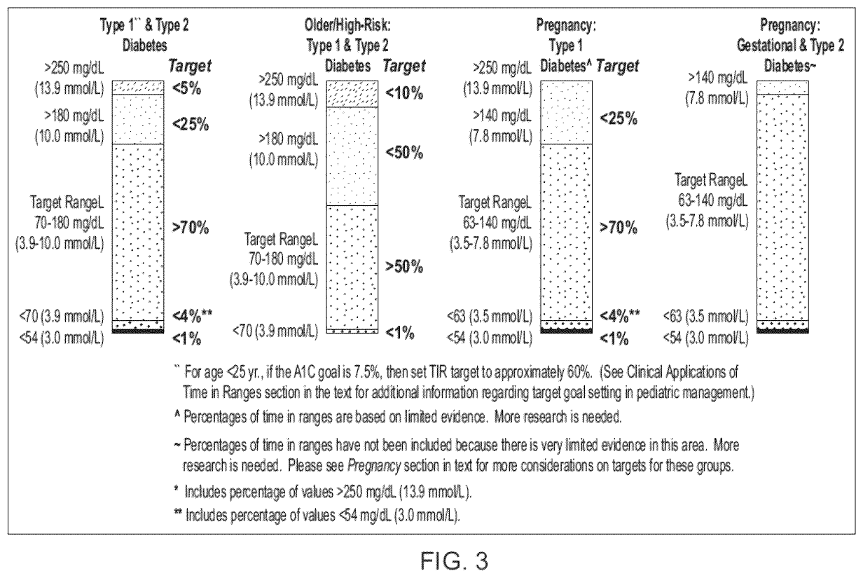

In this manuscript we introduce such a framework—a set of 35 clinically-similar clusters, such that any daily CGM profile can be assigned to a single CSC, which will approximate the clinical impression conveyed by the original CGM data. Two attributes of the set of CSCs are important: (1) It is finite and not too large—only 35 CSCs classify reasonably well the seemingly infinite multitude of daily CGM profiles, at least in terms of TIR metrics, and (2) It is fixed and does not need to be recalculated with new data. These attributes, finite and fixed, allow the set of CSCs to become the base for a number of clinical, computational, and algorithmic applications, essentially permitting table-lookup solutions to apparently complex treatment optimization problems. A non-exhaustive list of potential applications includes:
1. Data structuring, dimensionality reduction, and database indexing: the continuum of all possible daily CGM profiles, as clinically represented by AGP and TIR metrics, is reduced to a finite and fixed set of CSCs which can be used as input to decision support, clinical, and automated treatment algorithms. A database indexed by the structure defined by the CSCs will ensure fast and efficient search for subgroups of similar daily CGM profiles. This can facilitate features in decision support or AID systems, such as algorithms learning from a person's CGM patterns, and from the patterns of others stored in databases.
2. Distinguishing between health states and treatment modalities: in another application of the CSCs, we can envision that a 10 to 14-day CGM wear in home environment, perhaps accompanied by a predefined schedule of meals and physical activity, could achieve diagnostic results similar to those accepted in the clinical practice, based on the observed CSC pattern. Such an approach would greatly simplify the data collection, replace some common laboratory tests, and enable telemedicine approaches.
3. CGM pattern recognition and forecast: the transition probability matrix describing the evolution of a patient across the predefined set of CSCs, is a [mathematically] natural tool for observing disease or treatment progression. Pattern recognition, or recurrent behaviors, are reflected by patterns, or cycles, detected in the transition probabilities from one CSC to the next. Short- or long-term forecast of glycemic control is based on probability patterns or recurrent visits to a certain subset of CSCs. The latter is a subject of the theory of semi-Markov chains, which result from aggregation (lumping) of the state space into relevant subsets, characterized by random duration of time spent in each subset.
4. Tracking disease progression over time: glycemic control deterioration is signified by transitions into undesired CSCs and, conversely, a successful treatment optimization, or medication titration, is reflected by transition into clinically desirable CSCs. In a practical application, the set of CSCs would be labeled or ranked by clinical desirability, e.g. by TIR as in FIG. 1, and the label/rank of each CSC would be then fixed and used to guide treatment away from risks and towards optimal control. Automation of the treatment process is facilitated by the tabulation of thousands of daily CGM profiles into just a few CSCs.

The major finding of this manuscript is that any daily CGM profile can be approximated by one of 35 prefixed clinically-similar clusters. Approximation means that when a daily CGM profile is classified into a CSC, the CSC preserves the information carried by the original daily CGM profile, in terms of the time-in-range system of metrics. Thus, the CSCs expand, and to some extent complete, the interpretation of CGM data provided by the AGP/TIR system—while the AGP/TIR is a static snapshot of 14 days of data, the sequence of CSCs derived from the same data tracks the progression of glycemic control over time.

The time series of CSCs over 14 days illustrate how stable or volatile the glycemic control of the person is. In particular, this visualization allows us to see individual days where the glycemic control is quite different from what the person normally experiences. Because the set of CSCs condenses the clinical impressions conveyed by all possible daily CGM profiles into a finite actionable table, a host of clinical applications are enabled, including: database indexing, pattern recognition and tracking of treatment progression across a finite set of possibilities, table-lookup data interpretation for decision support and AID algorithms, or CGM replacement of common clinical tests.

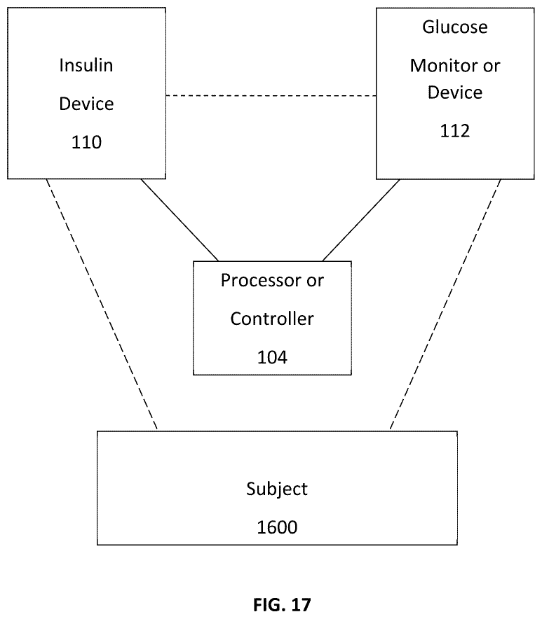

FIG. 17 is an exemplary high-level functional block diagram for an embodiment of the present invention, or an aspect of an embodiment of the present invention. As shown in FIG. 17, a processor 104 or controller communicates with the glucose monitor or data source 112, and optionally an insulin delivery device (e.g., other device 110). The glucose monitor or device communicates with the subject 1600 to monitor glucose levels of the subject 1600. The processor 104 or controller is configured to perform the required calculations. Optionally, the insulin delivery device communicates with the subject 1600 to deliver insulin to the subject 1600. The processor 104 or controller is configured to perform the required calculations. The glucose monitor and the insulin delivery device may be implemented as a separate device or as a single device. The processor 104 can be implemented locally in the glucose monitor, the insulin delivery device, or a standalone device (or in any combination of two or more of the glucose monitor, insulin device, or a stand along device). The processor 104 or a portion of the system can be located remotely such that the device is operated as a telemedicine device.

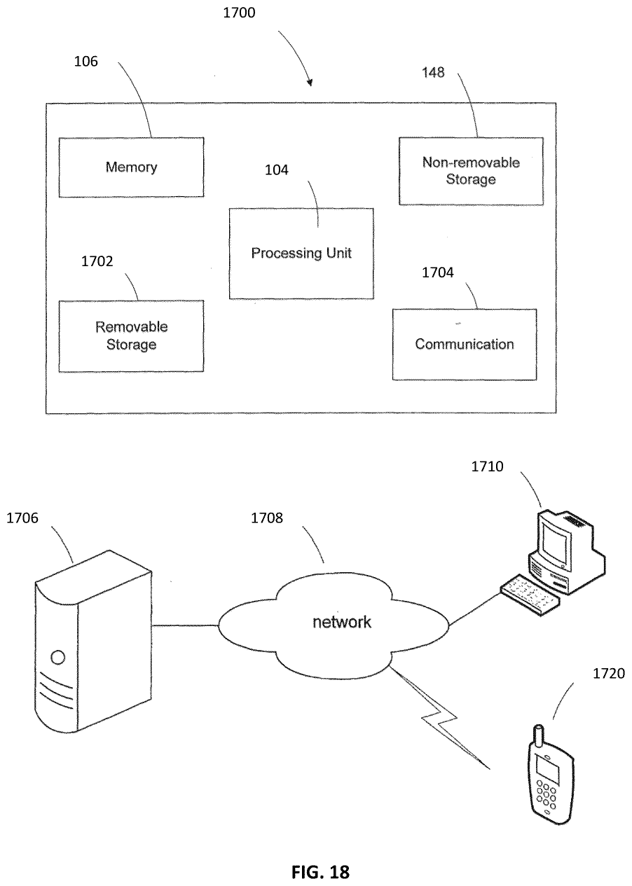

Referring to FIG. 18, in its most basic configuration, computing device 1700 typically includes at least one processor 104 and memory 106. Depending on the exact configuration and type of computing device, memory 106 can be volatile (such as RAM), non-volatile (such as ROM, flash memory, etc.) or some combination of the two.

Additionally, the computing device 1700 may also have other features and/or functionality. For example, the computing device 1700 could also include additional removable and/or non-removable storage including, but not limited to, magnetic or optical disks or tape, as well as writable electrical storage media. Such additional storage is the figure by removable storage 1702 and non-removable storage 1704. Computer storage media includes volatile and nonvolatile, removable and non-removable media implemented in any method or technology for storage of information such as computer readable instructions, data structures, program modules or other data. The memory, the removable storage and the non-removable storage are all examples of computer storage media. Computer storage media includes, but is not limited to, RAM, ROM, EEPROM, flash memory or other memory technology CDROM, digital versatile disks (DVD) or other optical storage, magnetic cassettes, magnetic tape, magnetic disk storage or other magnetic storage devices, or any other medium which can be used to store the desired information and which can accessed by the device. Any such computer storage media may be part of, or used in conjunction with, the device.

The computing device 1700 may also contain one or more communications connections 1708 that allow the device to communicate with other devices (e.g. other computing devices). The communications connections carry information in a communication media. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode, execute, or process information in the signal. By way of example, and not limitation, communication medium includes wired media such as a wired network or direct-wired connection, and wireless media such as radio, RF, infrared and other wireless media. As discussed above, the term computer readable media as used herein includes both storage media and communication media.

In addition to a stand-alone computing machine, embodiments of the invention can also be implemented on a network system comprising a plurality of computing devices that are in communication with a networking means, such as a network with an infrastructure or an ad hoc network. The network connection can be wired connections or wireless connections. As a way of example, FIG. 18 illustrates a network system in which embodiments of the invention can be implemented. In this example, the network system comprises computer 1706 (e.g. a network server), network connection means 1708 (e.g. wired and/or wireless connections), computer terminal 1710, and PDA (e.g. a smart-phone) 1720 (or other handheld or portable device, such as a cell phone, laptop computer, tablet computer, GPS receiver, mp3 player, handheld video player, pocket projector, etc. or handheld devices (or non portable devices) with combinations of such features). In an embodiment, it should be appreciated that the module 1706 may be glucose monitor device. In an embodiment, it should be appreciated that the module listed as 1706 may be a glucose monitor device, artificial pancreas, and/or an insulin device (or other interventional or diagnostic device). Any of the components may be multiple in number. The embodiments of the invention can be implemented in anyone of the devices of the system. For example, execution of the instructions or other desired processing can be performed on the same computing device 1700. Alternatively, an embodiment of the invention can be performed on different computing devices of the network system. For example, certain desired or required processing or execution can be performed on one of the computing devices of the network (e.g., server 1706 and/or glucose monitor device), whereas other processing and execution of the instruction can be performed at another computing device (e.g., terminal 1710) of the network system, or vice versa. In fact, certain processing or execution can be performed at one computing device (e.g. server 1706 and/or insulin device, artificial pancreas, or glucose monitor device (or other interventional or diagnostic device)); and the other processing or execution of the instructions can be performed at different computing devices that may or may not be networked. For example, the certain processing can be performed at terminal 1706, while the other processing or instructions are passed to a computing device 1700 where the instructions are executed. This scenario may be of particular value especially when the PDA device, for example, accesses to the network through computer terminal 1710 (or an access point in an ad hoc network). For another example, software to be protected can be executed, encoded or processed with one or more embodiments of the invention. The processed, encoded or executed software can then be distributed to customers. The distribution can be in a form of storage media (e.g., disk) or electronic copy.

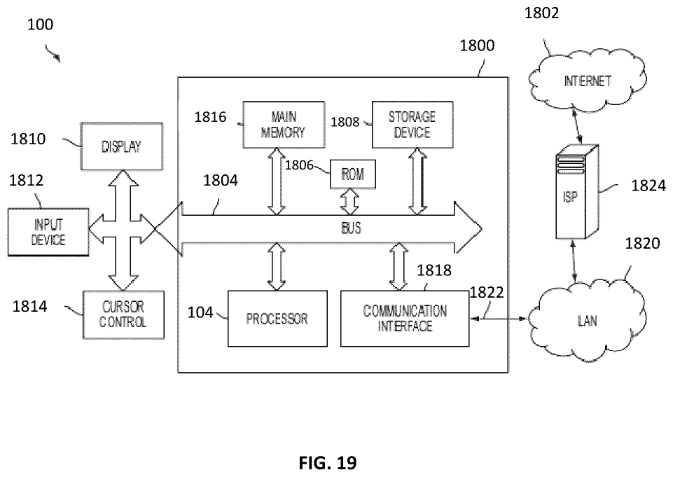

FIG. 19 is a block diagram that illustrates a system 100 including a computer system 1800 and the associated Internet 1802 connection upon which an embodiment may be implemented. Such configuration is typically used for computers (hosts) connected to the Internet 1802 and executing a server or a client (or a combination) software. A source computer such as laptop, an ultimate destination computer and relay servers, for example, as well as any computer or processor described herein, may use the computer system configuration and the Internet connection shown in FIG. 19. The system 1800 may be used as a portable electronic device such as a notebook/laptop computer, a media player (e.g., MP3 based or video player), a cellular phone, a Personal Digital Assistant (PDA), a glucose monitor device, an artificial pancreas, an insulin delivery device (or other interventional or diagnostic device), an image processing device (e.g., a digital camera or video recorder), and/or any other handheld computing devices, or a combination of any of these devices. Note that while FIG. 19 illustrates various components of a computer system, it is not intended to represent any particular architecture or manner of interconnecting the components; as such details are not germane to the present invention. It will also be appreciated that network computers, handheld computers, cell phones and other data processing systems which have fewer components or perhaps more components may also be used. The computer system of FIG. 19 may, for example, be an Apple Macintosh computer or Power Book, or an IBM compatible PC. Computer system 100 includes a bus 1804, an interconnect, or other communication mechanism for communicating information, and a processor 104, commonly in the form of an integrated circuit, coupled with bus 1804 for processing information and for executing the computer executable instructions. Computer system 100 also includes a main memory 106, such as a Random Access Memory (RAM) or other dynamic storage device, coupled to bus 1804 for storing information and instructions to be executed by processor 104.

Main memory 106 also may be used for storing temporary variables or other intermediate information during execution of instructions to be executed by processor 104. Computer system 100 further includes a Read Only Memory (ROM) 136 (or other non-volatile memory) or other static storage device coupled to bus 1804 for storing static information and instructions for processor 104. A storage device 1808, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 1804 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 100 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 100 may be coupled via bus 1804 to a display 1810, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 1812, including alphanumeric and other keys, is coupled to bus 1804 for communicating information and command selections to processor 104. Another type of user input device is cursor control 1814, such as a mouse, a trackball, or cursor direction keys for communicating direction information and command selections to processor 104 and for controlling cursor movement on display 1810. This input device typically has two degrees of freedom in two axes, a first axis (e.g., x) and a second axis (e.g., y), that allows the device to specify positions in a plane.

The computer system 1800 may be used for implementing the methods and techniques described herein. According to one embodiment, those methods and techniques are performed by computer system 1800 in response to processor 104 executing one or more sequences of one or more instructions contained in main memory 1816. Such instructions may be read into main memory 106 from another computer-readable medium, such as storage device 1808. Execution of the sequences of instructions contained in main memory 106 causes processor 104 to perform the process steps described herein. In alternative embodiments, hard-wired circuitry may be used in place of or in combination with software instructions to implement the arrangement. Thus, embodiments of the invention are not limited to any specific combination of hardware circuitry and software.

The term “computer-readable medium” (or “machine-readable medium”) as used herein is an extensible term that refers to any medium or any memory, that participates in providing instructions to a processor, (such as processor 104) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 1804. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 104 for execution. For example, the instructions may initially be carried on a magnetic disk of a remote computer.

The remote computer can load the instructions into its dynamic memory and send the instructions over a telephone line using a modem. A modem local to computer system 100 can receive the data on the telephone line and use an infra-red transmitter to convert the data to an infra-red signal. An infra-red detector can receive the data carried in the infra-red signal and appropriate circuitry can place the data on bus 1804. Bus 1804 carries the data to main memory 1816, from which processor 104 retrieves and executes the instructions. The instructions received by main memory 1816 may optionally be stored on storage device 1808 either before or after execution by processor 104.

Computer system 100 also includes a communication interface 1818 coupled to bus 1804. Communication interface 1818 provides a two-way data communication coupling to a network link 1822 that is connected to a local network 1820. For example, communication interface 1818 may be an Integrated Services Digital Network (ISDN) card or a modem to provide a data communication connection to a corresponding type of telephone line. As another non-limiting example, communication interface 1818 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (March 1999), “InternetworkingTechnologies Handbook”, Chapter 7: “Ethernet Technologies”, pages 7-1 to 7-38, which is incorporated in its entirety for all purposes as if fully set forth herein. In such a case, the communication interface 1818 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Feb. 20, 2004), which is incorporated in its entirety for all purposes as if fully set forth herein.

Wireless links may also be implemented. In any such implementation, communication interface 1818 sends and receives electrical, electromagnetic or optical signals that carry digital data streams representing various types of information.

Network link 1822 typically provides data communication through one or more networks to other data devices. For example, network link 1822 may provide a connection through local network 1820 to a host computer or to data equipment operated by an Internet Service Provider (ISP) 1824. ISP 1824 in turn provides data communication services through the world wide packet data communication network Internet 1802. Local network 1820 and Internet 1802 both use electrical, electromagnetic or optical signals that carry digital data streams. The signals through the various networks and the signals on the network link 1822 and through the communication interface 1818, which carry the digital data to and from computer system 100, are exemplary forms of carrier waves transporting the information.

A received code may be executed by processor 104 as it is received, and/or stored in storage device 1808, or other non-volatile storage for later execution. In this manner, computer system 100 may obtain application code in the form of a carrier wave.

The concept of identifying clinically-similar clusters of daily continuous glucose monitoring (CGM) profiles has been developed by the present inventor. The concept of performing the following: a) constructing and then fixing, a set of Clinically-Similar Clusters (CSCs), with the property that for any other daily continuous glucose monitoring (CGM) profile there is a Clinically-Similar Cluster (CSC) that approximates the time in ranges of said daily CGM profile, and b) determining an approximation of any daily CGM profile by a CSC has been developed by the present inventor.

As seen from the algorithm and methodology requirements discussed herein, the procedure is readily applicable into devices for identifying clinically-similar clusters of daily continuous glucose monitoring (CGM) profiles, and may be implemented and utilized with the related processors, networks, computer systems, internet, and components and functions according to the schemes disclosed herein. As seen from the algorithm and methodology requirements discussed herein, the procedure is readily applicable into devices for performing the following: a) constructing and then fixing, a set of Clinically-Similar Clusters (CSCs), with the property that for any other daily continuous glucose monitoring (CGM) profile there is a Clinically-Similar Cluster (CSC) that approximates the time in ranges of said daily CGM profile, and b) determining an approximation of any daily CGM profile by a CSC, and may be implemented and utilized with the related processors, networks, computer systems, internet, and components and functions according to the schemes disclosed herein.

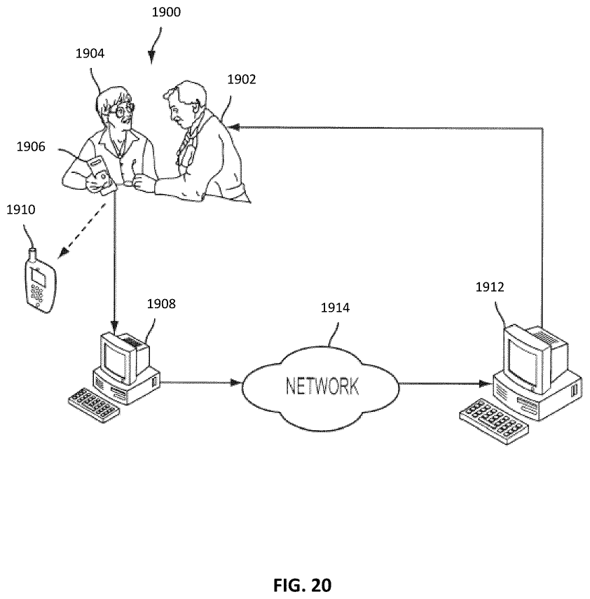

FIG. 20 illustrates a system in which one or more embodiments of the invention can be implemented using a network, or portions of a network or computers.

Although the present invention glucose monitor, artificial pancreas or insulin device (or other interventional or diagnostic device) may be practiced without a network. FIG. 20 diagrammatically illustrates an exemplary system in which examples of the invention can be implemented. In an embodiment the glucose monitor, artificial pancreas or insulin device (or other interventional or diagnostic device) may be implemented by the subject (or patient) locally at home or other desired location. However, in an alternative embodiment it may be implemented in a clinic setting or assistance setting. For instance, a clinic setup 1900 provides a place for doctors (e.g. 1902) or clinician/assistant to diagnose patients (e.g. 1904) with diseases related with glucose and related diseases and conditions. A glucose monitoring device 1906 can be used to monitor and/or test the glucose levels of the patient—as a standalone device. It should be appreciated that while only glucose monitor device 1906 is shown in the figure, the system of the invention and any component thereof may be used in the manner depicted by FIG. 20. The system or component may be affixed to the patient or in communication with the patient as desired or required. For example the system or combination of components thereof—including a glucose monitor device 1906 (or other related devices or systems such as a controller, and/or an artificial pancreas, an insulin pump (or other interventional or diagnostic device), or any other desired or required devices or components)—may be in contact, communication or affixed to the patient through tape or tubing (or other medical instruments or components) or may be in communication through wired or wireless connections. Such monitor and/or test can be short term (e.g. clinical visit) or long term (e.g. clinical stay or family). The glucose monitoring device outputs can be used by the doctor (clinician or assistant) for appropriate actions, such as insulin injection or food feeding for the patient, or other appropriate actions or modeling. Alternatively, the glucose monitoring device output can be delivered to computer terminal 1908 for instant or future analyses. The delivery can be through cable or wireless or any other suitable medium. The glucose monitoring device output from the patient can also be delivered to a portable device, such as PDA 1910. The glucose monitoring device outputs with improved accuracy can be delivered to a glucose monitoring center 1912 for processing and/or analyzing. Such delivery can be accomplished in many ways, such as network connection 1914, which can be wired or wireless.

In addition to the glucose monitoring device outputs, errors, parameters for accuracy improvements, and any accuracy related information can be delivered, such as to computer and/or glucose monitoring center 1912 for performing error analyses. This can provide a centralized accuracy monitoring, modeling and/or accuracy enhancement for glucose centers (or other interventional or diagnostic centers), due to the importance of the glucose sensors (or other interventional or diagnostic sensors or devices).

Examples of the invention can also be implemented in a standalone computing device associated with the target glucose monitoring device, artificial pancreas, and/or insulin device (or other interventional or diagnostic device.

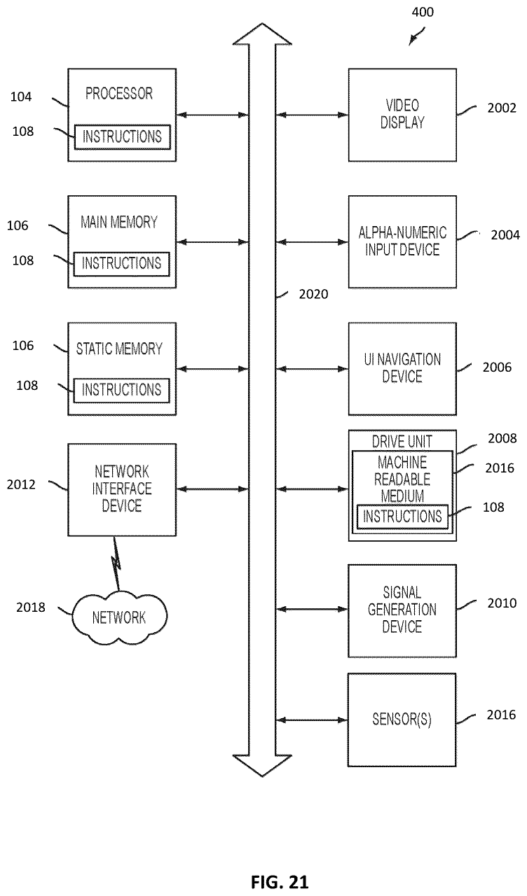

FIG. 21 is a block diagram illustrating an example of a machine upon which one or more aspects of embodiments of the present invention can be implemented.

Referring to FIG. 21, an aspect of an embodiment of the present invention includes, but not limited thereto, a system, method, and computer readable medium that provides the following: identifying clinically-similar clusters of daily continuous glucose monitoring (CGM) profiles, which illustrates a block diagram of an example machine 2000 upon which one or more embodiments (e.g., discussed methodologies) can be implemented (e.g., run).

Referring to FIG. 21, an aspect of an embodiment of the present invention includes, but not limited thereto, a system, method, and computer readable medium that provides the following: a) constructing and then fixing, a set of Clinically-Similar Clusters (CSCs), with the property that for any other daily continuous glucose monitoring (CGM) profile there is a Clinically-Similar Cluster (CSC) that approximates the time in ranges of said daily CGM profile, and b) determining an approximation of any daily CGM profile by a CSC, which illustrates a block diagram of an example machine 2000 upon which one or more embodiments (e.g., discussed methodologies) can be implemented (e.g., run).

Examples of machine 2000 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).)

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 2000) and software architectures that can be deployed in example embodiments.

In an example, the machine 2000 can operate as a standalone device or the machine 2000 can be connected (e.g., networked) to other machines.

In a networked deployment, the machine 2000 can operate in the capacity of either a server or a client machine in server-client network environments. In an example, machine 2000 can act as a peer machine in peer-to-peer (or other distributed) network environments. The machine 2000 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 2000. Further, while only a single machine 2000 is illustrated, the term “machine” shall also be taken to include any collection of machines that individually or jointly execute a set (or multiple sets) of instructions to perform any one or more of the methodologies discussed herein.

Example machine (e.g., computer system) 2000 can include a processor 104 (e.g., a central processing unit (CPU), a graphics processing unit (GPU) or both), a main memory 106 and a static memory 106, some or all of which can communicate with each other via a bus 2020. The machine 2000 can further include a display unit 2002, an alphanumeric input device 2004 (e.g., a keyboard), and a user interface (UI) navigation device 2006 (e.g., a mouse). In an example, the display unit 2002, input device 2004 and UI navigation device 2006 can be a touch screen display. The machine 2000 can additionally include a storage device (e.g., drive unit) 2008, a signal generation device 2010 (e.g., a speaker), a network interface device 2012, and one or more sensors 2014, such as a global positioning system (GPS) sensor, compass, accelerometer, or other sensor.

The storage device 2008 can include a machine readable medium 2016 on which is stored one or more sets of data structures or instructions 108 (e.g., software) embodying or utilized by any one or more of the methodologies or functions described herein. The instructions 108 can also reside, completely or at least partially, within the main memory 106, within static memory 106, or within the processor 104 during execution thereof by the machine 2000. In an example, one or any combination of the processor 104, the main memory 106, the static memory 106, or the storage device 2008 can constitute machine readable media.

While the machine readable medium 2016 is illustrated as a single medium, the term “machine readable medium” can include a single medium or multiple media (e.g., a centralized or distributed database, and/or associated caches and servers) that configured to store the one or more instructions 108. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, byway of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 108 can further be transmitted or received over a communications network 2018 using a transmission medium via the network interface device utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the invention described herein.

It should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

Additional descriptions of aspects of the present disclosure will now be provided with reference to the accompanying drawings. The drawings form a part hereof and show, by way of illustration, specific embodiments or examples.

The following references are incorporated herein by reference in their entireties.
1. M. D. Breton and B. P. Kovatchev, “One year real-world use of the control-IQ advanced hybrid closed-loop technology,” Diabetes technology & therapeutics, vol. 23, p. 601-608, 2021.
2. B. P. Kovatchev, “Metrics for glycaemic control—from HbA1c to continuous glucose monitoring,” Nature Reviews Endocrinology, vol. 13, p. 425-436, 2017.
3. B. Kovatchev and C. Cobelli, “Glucose variability: timing, risk analysis, and relationship to hypoglycemia in diabetes,” Diabetes Care, vol. 39, p. 502-510, 2016.
4. C. Cobelli, C. Dalla Man, G. Sparacino, L. Magni, G. De Nicolao and B. P. Kovatchev, “Diabetes: models, signals, and control,” IEEE reviews in biomedical engineering, vol. 2, p. 54-96, 2009.
5. B. P. Kovatchev, W. L. Clarke, M. Breton, K. Brayman and A. McCall, “Quantifying temporal glucose variability in diabetes via continuous glucose monitoring: mathematical methods and clinical application,” Diabetes technology & therapeutics, vol. 7, p. 849-862, 2005.
6. C. M. McDonnell, S. M. Donath, S. I. Vidmar, G. A. Werther and F. J. Cameron, “A novel approach to continuous glucose analysis utilizing glycemic variation,” Diabetes technology & therapeutics, vol. 7, p. 253-263, 2005.
7. P. A. Baghurst, “Calculating the mean amplitude of glycemic excursion from continuous glucose monitoring data: an automated algorithm,” Diabetes technology & therapeutics, vol. 13, p. 296-302, 2011.
8. C. Fabris, S. D. Patek and M. D. Breton, “Are risk indices derived from CGM interchangeable with SMBG-based indices?,” Journal of diabetes science and technology, vol. 10, p. 50-59, 2016.
9. A. L. McCall, D. J. Cox, J. Crean, M. Gloster and B. P. Kovatchev, “A novel analytical method for assessing glucose variability: using CGMS in type 1 diabetes mellitus,” Diabetes technology & therapeutics, vol. 8, p. 644-653, 2006.
10. D. Rodbard, “New and improved methods to characterize glycemic variability using continuous glucose monitoring,” Diabetes technology & therapeutics, vol. 11, p. 551-565, 2009.
11. D. Rodbard, “Interpretation of continuous glucose monitoring data: glycemic variability and quality of glycemic control,” Diabetes technology & therapeutics, vol. 11, p. S-55, 2009.
12. W. Clarke and B. Kovatchev, “Statistical tools to analyze continuous glucose monitor data,” Diabetes technology & therapeutics, vol. 11, p. S-45, 2009.
13. L. Magni, D. M. Raimondo, C. D. Man, M. Breton, S. Patek, G. De Nicolao, C. Cobelli and B. P. Kovatchev, “Evaluating the efficacy of closed-loop glucose regulation via control-variability grid analysis,” Journal of diabetes science and technology, vol. 2, p. 630-635, 2008.
14. T. Battelino, T. Danne, R. M. Bergenstal, S. A. Amiel, R. Beck, T. Biester, E. Bosi, B. A. Buckingham, W. T. Cefalu, K. L. Close and others, “Clinical targets for continuous glucose monitoring data interpretation: recommendations from the international consensus on time in range,” Diabetes care, vol. 42, p. 1593-1603, 2019.
15. R. S. Mazze, D. Lucido, O. Langer, K. Hartmann and D. Rodbard, “Ambulatory glucose profile: representation of verified self-monitored blood glucose data,” Diabetes Care, vol. 10, p. 111-117, 1987.
16. R. M. Bergenstal, A. J. Ahmann, T. Bailey, R. W. Beck, J. Bissen, B. Buckingham, L. Deeb, R. H. Dolin, S. K. Garg, R. Goland and others, Recommendations for standardizing glucose reporting and analysis to optimize clinical decision making in diabetes: the Ambulatory Glucose Profile (AGP), Mary Ann Liebert, Inc. 140 Huguenot Street, 3rd Floor New Rochelle, NY 10801 USA, 2013.
17. A. D. Association, “7. Diabetes Technology: Standards of Medical Care in Diabetes—2019,” Diabetes Care, vol. 42, pp. S71-S80, December 2018.
18. V. Fonseca and G. Grunberger, “Letter to the Editor,” Endocrine Practice, vol. 23, pp. 629-632, 2017.
19. B. Kovatchev, S. Anderson, D. Raghinaru, Y. Kudva, L. a. L. C. Laffel, J. Pinsker and others, “Randomized Controlled Trial of Mobile Closed-Loop Control,” Diabetes Care, vol. 43, no. 3, pp. 607-615, 2020.
20. S. Brown, B. Kovatchev, D. Raghinaru, J. Lum, B. Buckingham, Y. Kudva, L. Laffel and others, “Six-Month Randomized, Multicenter Trial of Closed-Loop Control in Type 1 Diabetes,” New England Journal of Medicine, vol. 381, no. 18, pp. 1707-1717, 2019.
21. R. Beck, T. Riddlesworth, K. Ruedy, A. Ahmann, R. Bergenstal, S. Haller, C. Kollman and others, “Effect of Continuous Glucose Monitoring on Glycemic Control in Adults With Type 1 Diabetes Using Insulin Injections: The DIAMOND Randomized Clinical Trial,” Journal of the American Medical Association, vol. 317, no. 4, pp. 371-378, 2017.
22. R. Beck, T. Riddlesworth, K. Ruedy, A. Ahmann, S. Haller, D. Kruger, J. McGill and others, “Continuous Glucose Monitoring Versus Usual Care in Patients With Type 2 Diabetes Receiving Multiple Daily Insulin Injections,” Annals of Internal Medicine, vol. 167, no. 6, pp. 365-374, 2017.
23. A. Bisio, S. Anderson, L. Norlander, G. O'Malley, J. Robic, S. Ogyaadu, L. Hsu and others, “Impact of a Novel Diabetes Support System on a Cohort of Individuals With Type 1 Diabetes Treated With Multiple Daily Injections: A Multicenter Randomized Study,” Diabetes Care, vol. 45, no. 1, pp. 186-193, 2022.
24. B. Kovatchev, L. Kollar, S. Anderson, C. Barnett, M. Breton, K. Carr, R. Gildersleeve and others, “Evening and overnight closed-loop control versus 24/7 continuous closed-loop control for type 1 diabetes: a randomised crossover trial,” The Lancet Digital Health, vol. 2, no. 2, pp. e64-e73, 2020.
25. L. Laffel, L. Kanapka, R. Beck, K. Bergamo, M. Clements, A. Criego, D. DeSalvo and others, “Effect of Continuous Glucose Monitoring on Glycemic Control in Adolescents and Young Adults With Type 1 Diabetes: A Randomized Clinical Trial,” Journal of the American Medical Association, vol. 323, no. 23, pp. 2388-2396, 2020.
26. M. Breton, L. Kanapka, R. Beck, L. Ekhlaspour, G. Forlenza, E. Cengiz, M. Schoelwer and others, “A randomized trial of closed-loop control in children with type 1 diabetes,” New England Journal of Medicine, vol. 383, no. 9, pp. 836-845, 2020.
27. V. Shah, S. DuBose, Z. Li, R. Beck, A. Peters, R. Weinstock, D. Kruger and others, “Continuous Glucose Monitoring Profiles in Healthy Nondiabetic Participants: A Multicenter Prospective Study,” The Journal of Clinical Endocrinology & Metabolism, vol. 104, no. 10, pp. 4356-4364, 2019.
28. M. Rickels, S. DuBose, E. Toschi, R. Beck, A. Verdejo, H. Wolpert, M. Cummins and others, “Mini-Dose Glucagon as a Novel Approach to Prevent Exercise-Induced Hypoglycemia in Type 1 Diabetes,” Diabetes Care, vol. 41, no. 9, pp. 1909-1916, 2018.
29. G. Aleppo, K. Ruedy, T. Riddlesworth, D. Kruger, A. Peters, I. Hirsch, R. Bergenstal and others, “REPLACE-BG: A Randomized Trial Comparing Continuous Glucose Monitoring With and Without Routine Blood Glucose Monitoring in Adults With Well-Controlled Type 1 Diabetes,” Diabetes Care, vol. 40, no. 4, pp. 538-545, 2017.
30. JDRF CGM Study Group, “Continuous glucose monitoring and intensive treatment of type 1 diabetes,” New England Journal of Medicine, vol. 359, no. 14, pp. 1464-1476, 2008.
31. L. Laffel, K. Harrington, A. Hanono, N. Naik, L. Ambler-Osborn, A. Schultz, L. DiMeglio and others, “A Randomized Clinical Trial Assessing Continuous Glucose Monitoring (CGM) Use With Standardized Education With or Without a Family Behavioral Intervention Compared With Fingerstick Blood Glucose Monitoring in Very Young Children With Type 1 Diabetes,” Diabetes Care, vol. 44, no. 2, pp. 464-472, 2021.
32. R. Weinstock, S. DuBose, R. Bergenstal, N. Chaytor, C. Peterson, B. Olson, M. Munshi and others, “Risk Factors Associated With Severe Hypoglycemia in Older Adults With Type 1 Diabetes,” Diabetes Care, vol. 39, no. 4, pp. 603-610, 2016.
33. A. Carlson, L. Kanapka, K. Miller, A. Ahmann, N. Chaytor, S. Fox, L. Kiblinger and others, “Hypoglycemia and Glycemic Control in Older Adults With Type 1 Diabetes: Baseline Results From the WISDM Study,” Journal of Diabetes Science and Technology, vol. 15, no. 3, pp. 582-592, 2021.
34. B. Lobo, L. Farhy, M. Shafiei and B. Kovatchev, “A data-driven approach to classifying daily continuous glucose monitoring (CGM) time series,” IEEE Transactions on Biomedical Engineering, vol. 62, no. 2, pp. 654-665, 2022.
35. P. Virtanen, R. Gommers, T. Oliphant, M. Haberland, T. Reddy, D. Cournapeau and others, “SciPy 1.0: Fundamental Algorithms for Scientific Computing in Python,” Nature Methods, vol. 17, pp. 261-272, 2020.
36. National Institute of Standards and Technology, “Weibull Distribution,” [Online]. Available: https://www.itl.nist.gov/div898/handbook/eda/section3/eda3668.htm. [Accessed 13 12 2021].
37. International Patent Application Serial No. PCT/US2022/032121, entitled “METHOD FOR STRUCTURING AND CLASSIFICATION OF CONTINUOUS GLUCOSE MONITORING (CGM) PROFILES”, filed Jun. 3, 2022.
38. U.S. Utility patent application Ser. No. 17/829,754, entitled “METHOD FOR STRUCTURING AND CLASSIFICATION OF CONTINUOUS GLUCOSE MONITORING (CGM) PROFILES”, filed Jun. 1, 2022.
39. U.S. Utility patent application Ser. No. 17/776,717, entitled “METHOD AND SYSTEM FOR MODEL-BASED TRACKING OF HEMOGLOBIN A1c FROM DAILY CONTINUOUS GLUCOSE MONITORING PROFILES”, filed May 13, 2022.
40. International Patent Application Serial No. PCT/US2020/060658, entitled “METHOD AND SYSTEM FOR MODEL-BASED TRACKING OF HEMOGLOBIN A1c FROM DAILY CONTINUOUS GLUCOSE MONITORING PROFILES”, filed Nov. 16, 2020; Publication No. WO 2021/097396, May 20, 2021.
41. U.S. Utility patent application Ser. No. 17/776,056, entitled “SYSTEM, METHOD AND COMPUTER READABLE MEDIUM FOR COMPRESSING CONTINUOUS GLUCOSE MONITOR DATA”, filed May 11, 2022.
42. International Patent Application Serial No. PCT/US2020/060366, entitled “SYSTEM, METHOD AND COMPUTER READABLE MEDIUM FOR COMPRESSING CONTINUOUS GLUCOSE MONITOR DATA”, filed Nov. 13, 2020; Publication No. WO 2021/097178, May 20, 2021.
43. U.S. Utility patent application Ser. No. 17/694,870, entitled “INSULIN MONITORING AND DELIVERY SYSTEM AND METHOD FOR CGM BASED FAULT DETECTION AND MITIGATION VIA METABOLIC STATE TRACKING”, filed Mar. 15, 2022; Publication No. US 2022-0203020 A1, Jun. 30, 2022.
44. U.S. Utility patent application Ser. No. 15/580,935, entitled “INSULIN MONITORING AND DELIVERY SYSTEM AND METHOD FOR CGM BASED FAULT DETECTION AND MITIGATION VIA METABOLIC STATE TRACKING”, filed Dec. 8, 2017; U.S. Pat. No. 11,311,665, issued Apr. 26, 2022.
45. International Patent Application Serial No. PCT/US2016/036729, entitled “INSULIN MONITORING AND DELIVERY SYSTEM AND METHOD FOR CGM BASED FAULT DETECTION AND MITIGATION VIA METABOLIC STATE TRACKING”, filed Jun. 9, 2016; Publication No. WO 2016/201120, Dec. 15, 2016.
46. U.S. Utility patent application Ser. No. 17/683,676, entitled “System, Method and Computer Readable Medium for Dynamical Tracking of the Risk for Hypoglycemia in Type 1 and Type 2 Diabetes”, filed Mar. 1, 2022; Publication No. US 2022-0262519 A1, Aug. 18, 2022.
47. U.S. Utility patent application Ser. No. 15/958,257, entitled “System, Method and Computer Readable Medium for Dynamical Tracking of the Risk for Hypoglycemia in Type 1 and Type 2 Diabetes”, filed Apr. 20, 2018; U.S. Pat. No. 11,289,201, issued Mar. 29, 2022.
48. International Patent Application Serial No. PCT/US2016/058234, entitled “System, Method and Computer Readable Medium for Dynamical Tracking of the Risk for Hypoglycemia in Type 1 and Type 2 Diabetes”, filed Oct. 21, 2016; Publication No. WO 2017/070553, Apr. 27, 2017.
49. International Patent Application Serial No. PCT/US2022/017489, entitled “METHOD AND SYSTEM FOR QUANTITATIVE PHYSIOLOGICAL ASSESSMENT AND PREDICTION OF CLINICAL SUBTYPES OF GLUCOSE METABOLISM DISORDERS”, filed Feb. 23, 2022; Publication No. WO 2022/182736, Sep. 1, 2022.
50. International Patent Application Serial No. PCT/US2022/017449, entitled “METHOD AND SYSTEM FOR MAPPING INDIVIDUALIZED METABOLIC PHENOTYPE TO A DATABASE IMAGE FOR OPTIMIZING CONTROL OF CHRONIC METABOLIC CONDITIONS”, filed Feb. 23, 2022; Publication No. WO2022182709, Sep. 1, 2022.
51. U.S. Utility patent application Ser. No. 17/590,659, entitled “System, Method, and Computer Simulation Environment for In Silico Trials in Pre-Diabetes and Type 2 Diabetes”, filed Feb. 1, 2022; Publication No. US 2022-0230762 A1, Jul. 21, 2022.
52. U.S. Utility patent application Ser. No. 13/380,839, entitled “System, Method, and Computer Simulation Environment for In Silico Trials in Pre-Diabetes and Type 2 Diabetes”, filed Dec. 25, 2011; U.S. Pat. No. 11,238,990, issued Feb. 1, 2022.
53. International Patent Application Serial No. PCT/US2010/040097, entitled “System, Method, and Computer Simulation Environment for In Silico Trials in Prediabetes and Type 2 Diabetes”, filed Jun. 25, 2010; Publication No. WO 2010/151834, Dec. 29, 2010.
54. International Patent Application Serial No. PCT/US2021/045936, entitled “METHOD AND SYSTEM FOR GENERATING A USER TUNABLE REPRESENTATION OF GLUCOSE HOMEOSTASIS IN TYPE 1 DIABETES BASED ON AUTOMATED RECEIPT OF THERAPY PROFILE DATA”, filed Aug. 13, 2021; Publication No. WO 2022/036214, Feb. 17, 2022.
55. U.S. Utility patent application Ser. No. 17/339,153, entitled “Method and System for the Safety, Analysis, and Supervision of Insulin Pump Action and Other Modes of Insulin Delivery in Diabetes”, filed Jun. 4, 2021; Publication No. US 2021-0313036 A1, Oct. 7, 2021.
56. U.S. Utility patent application Ser. No. 13/634,040, entitled “Method and System for the Safety, Analysis, and Supervision of Insulin Pump Action and Other Modes of Insulin Delivery in Diabetes”, filed Sep. 11, 2012; U.S. Pat. No. 11,069,434, issued Jul. 20, 2021.
57. International Patent Application Serial No. PCT/US2011/028163, entitled “Method and System for the Safety, Analysis, and Supervision of Insulin Pump Action and Other Modes of Insulin Delivery in Diabetes”, filed Mar. 11, 2011; Publication No. WO 2011/112974, Sep. 15, 2011.
58. U.S. Utility patent application Ser. No. 17/333,161, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed May 28, 2021; Publication No. US 2021-0282677 A1, Sep. 16, 2021.
59. U.S. Utility patent application Ser. No. 17/070,245, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed Oct. 14, 2020; Publication No. US 2021/0038132 A1, Feb. 11, 2021.
60. U.S. Utility patent application Ser. No. 15/669,111, entitled “METHOD, SYSTEM AND COMPUTER PROGRAM PRODUCT FOR CGM-BASED PREVENTION OF HYPOGLYCEMIA VIA HYPOGLYCEMIA RISK ASSESSMENT AND SMOOTH REDUCTION INSULIN DELIVERY”, filed Aug. 4, 2017; U.S. Pat. No. 10,842,419, issued Nov. 24, 2020.
61. U.S. Utility patent application Ser. No. 14/015,831, entitled “CGM-Based Prevention of Hypoglycemia Via Hypoglycemia Risk Assessment and Smooth Reduction of Insulin Delivery”, filed Aug. 30, 2013; U.S. Pat. No. 9,750,438, issued Sep. 5, 2017.
62. U.S. Utility patent application Ser. No. 13/203,469, entitled “CGM-Based Prevention of Hypoglycemia via Hypoglycemia Risk Assessment and Smooth Reduction Insulin Delivery”, filed Aug. 25, 2011; U.S. Pat. No. 8,562,587, issued Oct. 22, 2013.
63. International Patent Application Serial No. PCT/US2010/025405, entitled “CGM-BASED PREVENTION OF HYPOGLYCEMIA VIA HYPOGLYCEMIA RISK ASSESSMENT AND SMOOTH REDUCTION INSULIN DELIVERY”, filed Feb. 25, 2010; Publication No. WO 2010/099313 A1, Sep. 2, 2010.
64. U.S. Utility patent application Ser. No. 17/181,888, entitled “SYSTEMS OF CENTRALIZED DATA EXCHANGE FOR MONITORING AND CONTROL OF BLOOD 65. GLUCOSE”, filed Feb. 22, 2021; Publication No. US 2021-0169409 A1, Jun. 10, 2021.
66. U.S. Utility patent application Ser. No. 15/109,682, entitled “SYSTEMS OF CENTRALIZED DATA EXCHANGE FOR MONITORING AND CONTROL OF BLOOD GLUCOSE”, filed Jul. 5, 2016; U.S. Pat. No. 10,925,536, issued Feb. 23, 2021.
67. International Patent Application Serial No. PCT/US2015/010167, entitled “Central Data Exchange Node For System Monitoring and Control of Blood Glucose Levels in Diabetic Patients”, filed Jan. 5, 2015; Publication No. WO2015103543, Jul. 9, 2015.
68. U.S. Utility patent application Ser. No. 17/180,301, entitled “INFLUENCING END-STAGE RENAL DISEASE OUTCOMES THROUGH PREDICTING PHYSIOLOGICAL PARAMETERS AND DETERMINING DOSING RECOMMENDATIONS”, filed Feb. 19, 2021; Publication No. US 2021-0257096 A1, Aug. 19, 2021.
69. U.S. Utility patent application Ser. No. 17/156,169, entitled “Method, System and Computer Program Product for Real-Time Detection of Sensitivity Decline in Analyte Sensors”, filed Jan. 22, 2021; Publication No. US 2021-0218481 A1, Jul. 15, 2021.
70. U.S. Utility patent application Ser. No. 15/866,384, entitled “Method, System and Computer Program Product for Real-Time Detection of Sensitivity Decline in Analyte Sensors”, filed Jan. 9, 2018; U.S. Pat. No. 10,903,914, issued Jan. 26, 2021.
71. U.S. Utility patent application Ser. No. 14/266,612, entitled “Method, System and Computer Program Product for Real-Time Detection of Sensitivity Decline in Analyte Sensors”, filed Apr. 30, 2014; U.S. Pat. No. 9,882,660, issued Jan. 30, 2018.
72. U.S. Utility patent application Ser. No. 13/418,305, entitled “Method, System and Computer Program Product for Real-Time Detection of Sensitivity Decline in Analyte Sensors”, filed Mar. 12, 2012; U.S. Pat. No. 8,718,958, issued May 6, 2014.
73. International Patent Application Serial No. PCT/US2007/082744, entitled “Method, System and Computer Program Product for Real-Time Detection of Sensitivity Decline in Analyte Sensors”, filed Oct. 26, 2007; Publication No. WO/2008/052199, May 2, 2008.
74. U.S. Utility patent application Ser. No. 11/925,689, entitled “Method, System and Computer Program Product for Real-Time Detection of Sensitivity Decline in Analyte Sensors”, filed Oct. 26, 2007; U.S. Pat. No. 8,135,548, issued Mar. 13, 2012.
75. U.S. Utility patent application Ser. No. 17/132,604, entitled “ACCURACY CONTINUOUS GLUCOSE MONITORING METHOD, SYSTEM, AND DEVICE”, filed Dec. 23, 2020; Publication No. US 2021-0113122 A1, Apr. 22, 2021.
76. U.S. Utility patent application Ser. No. 15/510,878, entitled “ACCURACY CONTINUOUS GLUCOSE MONITORING METHOD, SYSTEM, AND DEVICE”, filed Mar. 13, 2017; U.S. Pat. No. 10,881,334, issued Jan. 5, 2021.
77. International Patent Application Serial No. PCT/US2015/045340, entitled “IMPROVED ACCURACY CONTINUOUS GLUCOSE MONITORING METHOD, SYSTEM, AND DEVICE”, filed Aug. 14, 2015; Publication No. WO2016025874, Feb. 18, 2016.
78. U.S. Utility patent application Ser. No. 16/789,901, entitled “Unified Platform For Monitoring and Control of Blood Glucose Levels in Diabetic Patients”, filed Feb. 13, 2020; Publication No. US-2020-0214629-A1, Jul. 9, 2020.
79. U.S. Utility patent application Ser. No. 14/128,922, entitled “Unified Platform For Monitoring and Control of Blood Glucose Levels in Diabetic Patients”, filed Dec. 23, 2013; U.S. Pat. No. 10,610,154, issued Apr. 7, 2020.
80. International Patent Application Serial No. PCT/US2012/043910, entitled “Unified Platform For Monitoring and Control of Blood Glucose Levels in Diabetic Patients”, filed Jun. 23, 2012; Publication No. WO 2012/178134, Dec. 27, 2012.
81. U.S. Utility patent application Ser. No. 16/588,881, entitled “Tracking the Probability for Imminent Hypoglycemia in Diabetes from Self-Monitoring Blood Glucose (SMBG) Data”, filed Sep. 30, 2019; Publication No. US-2020-0066410-A1, Feb. 27, 2020.
82. U.S. Utility patent application Ser. No. 13/394,091, entitled “Tracking the Probability for Imminent Hypoglycemia in Diabetes from Self-Monitoring Blood Glucose (SMBG) Data”, filed Mar. 2, 2012; U.S. Pat. No. 10,431,342, issued Oct. 1, 2019.
83. International Patent Application Serial No. PCT/US2010/047711, entitled “Tracking the Probability for Imminent Hypoglycemia in Diabetes from Self-Monitoring Blood Glucose (SMBG) Data”, filed Sep. 2, 2010; Publication No. WO 2011/028925, Mar. 10, 2011.
84. U.S. Utility patent application Ser. No. 16/546,335, entitled “System Coordinator and Modular Architecture for Open-Loop and Closed-Loop Control of Diabetes”, filed Aug. 21, 2019; Publication No. US-2019-0374137-A1, Dec. 12, 2019.
85. U.S. Utility patent application Ser. No. 13/322,943, entitled “System Coordinator and Modular Architecture for Open-Loop and Closed-Loop Control of Diabetes”, filed Nov. 29, 2011; U.S. Pat. No. 10,420,489, issued Sep. 24, 2019.
86. International Patent Application Serial No. PCT/US2010/036629, entitled “System Coordinator and Modular Architecture for Open-Loop and Closed-Loop Control of Diabetes”, filed May 28, 2010; Publication No. WO 2010/138848, Dec. 2, 2010.
87. U.S. Utility patent application Ser. No. 16/451,766, entitled “TRACKING CHANGES IN AVERAGE GLYCEMIA IN DIABETICS”, filed Jun. 25, 2019; Publication No. US-2019-0318801-A1, Oct. 17, 2019.
88. U.S. Utility patent application Ser. No. 14/769,638, entitled “METHOD AND SYSTEM FOR MODEL-BASED TRACKING OF CHANGES IN AVERAGE GLYCEMIA IN DIABETES”, filed Aug. 21, 2015; U.S. Pat. No. 10,332,615, issued Jun. 25, 2019.
89. International Patent Application Serial No. PCT/US2014/017754, entitled “METHOD AND SYSTEM FOR MODEL-BASED TRACKING OF CHANGES IN AVERAGE GLYCEMIA IN DIABETES”, filed Feb. 21, 2014; Publication No. WO 2014/130841, Aug. 28, 2014.
90. U.S. Utility patent application Ser. No. 17/180,301, entitled “INFLUENCING END-STAGE RENAL DISEASE OUTCOMES THROUGH PREDICTING PHYSIOLOGICAL PARAMETERS AND DETERMINING DOSING RECOMMENDATIONS”, filed Feb. 19, 2021; Publication No. US 2021-0257096 A1, Aug. 19, 2021.
91. U.S. Utility patent application Ser. No. 16/126,879, entitled “Method, System and Computer Program Product for Evaluation of Insulin Sensitivity, Insulin/Carbohydrate Ratio, and Insulin Correction Factors in Diabetes from Self-Monitoring Data”, filed Sep. 10, 2018; Publication No. US-2019-0019571-A1, Jan. 17, 2019.
92. U.S. Utility patent application Ser. No. 12/665,149, entitled “Method, System and Computer Program Product for Evaluation of Insulin Sensitivity, Insulin/Carbohydrate Ratio, and Insulin Correction Factors in Diabetes from Self-Monitoring Data”, filed Dec. 17, 2009; Publication No. 2010/0198520, Aug. 5, 2010.
93. International Patent Application Serial No. PCT/US2008/069416, entitled “Method, System and Computer Program Product for Evaluation of Insulin Sensitivity, Insulin/Carbohydrate Ratio, and Insulin Correction Factors in Diabetes from Self-Monitoring Data”, filed Jul. 8, 2008; Publication No. WO 2009/009528, Jan. 15, 2009.
94. U.S. Utility patent application Ser. No. 15/580,915, entitled “System and Method for Tracking Changes in Average Glycemia in Diabetics”, filed Dec. 8, 2017; Publication No. US-2018-0313815-A1, Nov. 1, 2018.
95. International Patent Application Serial No. PCT/US2016/036481, entitled “System and Method for Tracking Changes in Average Glycemia in Diabetics”, filed Jun. 8, 2016; Publication No. WO2016200970, Dec. 15, 2016.
96. U.S. Utility patent application Ser. No. 14/902,731, entitled “SIMULATION OF ENDOGENOUS AND EXOGENOUS GLUCOSE/INSULIN/GLUCAGON INTERPLAY IN TYPE 1 DIABETIC PATIENTS”, filed Jan. 4, 2016; U.S. Pat. No. 10,169,544, issued Jan. 1, 2019.
97. International Patent Application Serial No. PCT/US2014/045393, entitled “SIMULATION OF ENDOGENOUS AND EXOGENOUS GLUCOSE/INSULIN/GLUCAGON INTERPLAY IN TYPE 1 DIABETIC PATIENTS”, filed Jul. 3, 2014; Publication No. WO2015003124, Jan. 8, 2015.
98. U.S. Utility patent application Ser. No. 14/799,329, entitled “ACCURACY OF CONTINUOUS GLUCOSE SENSORS”, filed Jul. 14, 2015; U.S. Pat. No. 10,194,850, issued Feb. 5, 2019.
99. U.S. Utility patent application Ser. No. 12/065,257, entitled “Accuracy of Continuous Glucose Sensors”, filed Feb. 28, 2008; Publication No. 2008/0314395, Dec. 25, 2008.
100. International Patent Application Serial No. PCT/US2006/033724, entitled “Method for Improvising Accuracy of Continuous Glucose Sensors and a Continuous Glucose Sensor Using the Same”, filed Aug. 29, 2006; Publication No. WO07027691, Mar. 8, 2007.
101. U.S. Utility patent application Ser. No. 14/419,375, entitled “COMPUTER SIMULATION FOR TESTING AND MONITORING OF TREATMENT STRATEGIES FOR STRESS HYPERGLYCEMIA”, filed Feb. 3, 2015; U.S. Pat. No. 10,438,700, issued Oct. 8, 2019.
102. International Patent Application Serial No. PCT/US2013/053664, entitled “COMPUTER SIMULATION FOR TESTING AND MONITORING OF TREATMENT STRATEGIES FOR STRESS HYPERGLYCEMIA”, filed Aug. 5, 2013; Publication No. WO 2014/022864, Feb. 6, 2014.
103. U.S. Utility patent application Ser. No. 14/241,383, entitled “Method, System and Computer Readable Medium for Adaptive Advisory Control of Diabetes”, filed Feb. 26, 2014; U.S. Pat. No. 11,024,429, issued Jun. 1, 2021.
104. International Patent Application Serial No. PCT/US2012/052422, entitled “Method, System and Computer Readable Medium for Adaptive Advisory Control of Diabetes”, filed Aug. 26, 2012; Publication No. WO 2013/032965, Mar. 7, 2013.
105. U.S. Utility patent application Ser. No. 14/128,811, entitled “Methods and Apparatus for Modular Power Management and Protection of Critical Services in Ambulatory Medical Devices”, filed Dec. 23, 2013; U.S. Pat. No. 9,430,022, issued Aug. 30, 2016.
106. International Patent Application Serial No. PCT/US2012/043883, entitled “Methods and Apparatus for Modular Power Management and Protection of Critical Services in Ambulatory Medical Devices”, filed Jun. 22, 2012; Publication No. WO 2012/178113, Dec. 27, 2012.
107. U.S. Utility patent application Ser. No. 29/467,039, entitled “Alarm Clock Display of Personal Blood Glucose Level”, filed Sep. 13, 2013.
108. International Patent Application Serial No. PCT/US2013/042745, entitled “INSULIN-PRAMLINTIDE COMPOSITIONS AND METHODS FOR MAKING AND USING THEM”, filed May 24, 2013; Publication No. WO 2013/177565, Nov. 28, 2013.
109. U.S. Utility patent application Ser. No. 13/637,359, entitled “METHOD, SYSTEM, AND COMPUTER PROGRAM PRODUCT FOR IMPROVING THE ACCURACY OF GLUCOSE SENSORS USING INSULIN DELIVERY OBSERVATION IN DIABETES”, filed Sep. 25, 2012; U.S. Pat. No. 9,398,869, issued Jul. 26, 2016.
110. International Patent Application Serial No. PCT/US2011/029793, entitled “METHOD, SYSTEM, AND COMPUTER PROGRAM PRODUCT FOR IMPROVING THE ACCURACY OF GLUCOSE SENSORS USING INSULIN DELIVERY OBSERVATION IN DIABETES”, filed Mar. 24, 2011; Publication No. WO 2011/119832, Sep. 29, 2011.
111. U.S. Utility patent application Ser. No. 13/393,647, entitled “System, Method and Computer Program Product for Adjustment of Insulin Delivery (AID) in Diabetes Using Nominal Open-Loop Profiles”, filed Mar. 1, 2012; Publication No. 2012/0245556, Sep. 27, 2012.
112. International Patent Application Serial No. PCT/US2010/047386, entitled “System, Method and Computer Program Product for Adjustment of Insulin Delivery (AID) in Diabetes Using Nominal Open-Loop Profiles”, filed Aug. 31, 2010; Publication No. WO 2011/028731, Mar. 10, 2011.
113. U.S. Utility patent application Ser. No. 13/131,467, entitled “Method, System, and Computer Program Product for Tracking of Blood Glucose Variability in Diabetes”, filed May 26, 2011; U.S. Pat. No. 9,317,657, issued Apr. 19, 2016.
114. International Patent Application Serial No. PCT/US2009/065725, entitled “Method, System, and Computer Program Product for Tracking of Blood Glucose Variability in Diabetes”, filed Nov. 24, 2009; Publication No. WO 2010/062898, Jun. 3, 2010.
115. U.S. Utility patent application Ser. No. 12/975,580, entitled “Method, System, and Computer Program Product for the Evaluation of Glycemic Control in Diabetes from Self-Monitoring Data”, filed Dec. 22, 2010; Publication No. 2012/0004512, Jan. 5, 2012.
116. U.S. Utility patent application Ser. No. 11/305,946, entitled “Method, System, and Computer Program Product for the Evaluation of Glycemic Control in Diabetes from Self-Monitoring Data”, filed Dec. 19, 2005; U.S. Pat. No. 7,874,985, issued Jan. 25, 2011.
117. U.S. Utility patent application Ser. No. 10/240,228, entitled “Method, System, and Computer Program Product for the Evaluation of Glycemic Control in Diabetes from Self-Monitoring Data”, filed Sep. 26, 2002; U.S. Pat. No. 7,025,425, issued Apr. 11, 2006.
118. International Patent Application Serial No. PCT/US2001/009884, entitled “Method, System, and Computer Program Product for the Evaluation of Glycemic Control in Diabetes”, filed Mar. 29, 2001; Publication No. WO 01/72208, Oct. 4, 2001.
119. U.S. Utility patent application Ser. No. 12/664,444, entitled “Method, System and Computer Simulation Environment for Testing of Monitoring and Control Strategies in Diabetes”, filed Dec. 14, 2009; U.S. Pat. No. 10,546,659, issued Jan. 28, 2020.
120. International Patent Application Serial No. PCT/US2008/067725, entitled “Method, System and Computer Simulation Environment for Testing of Monitoring and Control Strategies in Diabetes”, filed Jun. 20, 2008; Publication No. WO 2008/157781, Dec. 24, 2008.
121. U.S. Utility patent application Ser. No. 12/516,044, entitled “Method, System, and Computer Program Product for the Detection of Physical Activity by Changes in Heart Rate, Assessment of Fast Changing Metabolic States, and Applications of Closed and Open Control Loop in Diabetes”, filed May 22, 2009; U.S. Pat. No. 8,585,593, issued Nov. 19, 2013.
122. International Patent Application Serial No. PCT/US2007/085588, entitled “Method, System, and Computer Program Product for the Detection of Physical Activity by Changes in Heart Rate, Assessment of Fast Changing Metabolic States, and Applications of Closed and Open Control Loop in Diabetes”, filed Nov. 27, 2007; Publication No. WO2008/067284, Jun. 5, 2008.
123. U.S. Utility patent application Ser. No. 12/159,891, entitled “Method, System and Computer Program Product for Evaluation of Blood Glucose Variability in Diabetes from Self-Monitoring Data”, filed Jul. 2, 2008; U.S. Pat. No. 11,355,238, issued Jun. 7, 2022.
124. International Patent Application Serial No. PCT/US2007/000370, entitled “Method, System and Computer Program Product for Evaluation of Blood Glucose Variability in Diabetes from Self-Monitoring Data”, filed Jan. 5, 2007; Publication No. WO07081853, Jul. 19, 2007.
125. U.S. Utility patent application Ser. No. 11/943,226, entitled “Systems, Methods and Computer Program Codes for Recognition of Patterns of Hyperglycemia and Hypoglycemia, Increased Glucose Variability, and Ineffective Self-Monitoring in Diabetes”, filed Nov. 20, 2007; Publication No. 2008/0154513, Jun. 26, 2008.
126. U.S. Utility patent application Ser. No. 11/578,831, entitled “Method, System and Computer Program Product for Evaluating the Accuracy of Blood Glucose Monitoring Sensors/Devices”, filed Oct. 18, 2006; U.S. Pat. No. 7,815,569, issued Oct. 19, 2010.
127. International Patent Application Serial No. US2005/013792, entitled “Method, System and Computer Program Product for Evaluating the Accuracy of Blood Glucose Monitoring Sensors/Devices”, filed Apr. 21, 2005; Publication No. WO05106017, Nov. 10, 2005.
128. U.S. Utility patent application Ser. No. 10/524,094, entitled “Method, System, And Computer Program Product For The Processing Of Self-Monitoring Blood Glucose (SMBG) Data To Enhance Diabetic Self-Management”, filed Feb. 9, 2005; U.S. Pat. No. 8,538,703, issued Sep. 17, 2013.
129. International Patent Application Serial No. PCT/US2003/025053, entitled “Managing and Processing Self-Monitoring Blood Glucose”, filed Aug. 8, 2003; Publication No. WO 2004/015539, Feb. 19, 2004.
130. International Patent Application Serial No. PCT/US2002/005676, entitled “METHOD AND APPARATUS FOR THE EARLY DIAGNOSIS OF SUBACUTE, POTENTIALLY CATASTROPHIC ILLNESS”, filed Feb. 27, 2002; Publication No. WO02/67776, Sep. 6, 2002.
131. U.S. Utility patent application Ser. No. 09/793,653, entitled “METHOD AND APPARATUS FOR THE EARLY DIAGNOSIS OF SUBACUTE, POTENTIALLY CATASTROPHIC ILLNESS”, filed Feb. 27, 2001; U.S. Pat. No. 6,804,551, issued Oct. 12, 2004.
132. U.S. Utility patent application Ser. No. 10/069,674, entitled “Method and Apparatus for Predicting the Risk of Hypoglycemia”, filed Feb. 22, 2002; U.S. Pat. No. 6,923,763, issued Aug. 2, 2005.
133. International Patent Application Serial No. US00/22886, entitled “METHOD AND APPARATUS FOR PREDICTING THE RISK OF HYPOGLYCEMIA”, filed Aug. 21, 2000; Publication No. WO01/13786, Mar. 1, 2001.

What is claimed is:

1. A system for processing glucose data by efficient glucose database management, the system comprising:
a physical data store containing glucose measurement data and a representation for at least one cluster of the glucose measurement data, wherein the representation approximates a glycemic profile vector array for a cluster of multiple glucose profiles segmented by plural time ranges; and
a processor and computer memory configured with instructions stored thereon that when executed will cause the processor to:
receive glucose measurements;
convert the glucose measurements into vectorial form;
search the physical data store by comparing a newly received glucose measurement to a centroid of a cluster using a similarity metric;
classify the newly received glucose measurement with a cluster having a matched similarity metric based on the comparing; and
ascribe treatment to the newly received glucose measurement.

2. The system of claim 1, wherein instructions cause the processor to one or more of:
store the classification of the newly received glucose measurement in a data store that is in communication with one or more of a predictive modeling system, a decision support system, an insulin delivery system, an insulin monitoring system, or an automated control system configured to use the classification as input;
transmit the classification of the newly received glucose measurement to one or more of a predictive modeling system, a decision support system, an insulin delivery system, an insulin monitoring system, or an automated control system configured to use the classification as input; or
monitor, analyze, or influence a concentration of glucose levels in a fluid using the classification of the newly received glucose measurement.

3. The system of claim 1, wherein:
instructions cause the processor to receive the glucose measurements from a glucose measurement device.

4. The system of claim 3, comprising:
the glucose measurement device.

5. The system of claim 2, comprising:
the data store that is in communication with one or more of the predictive modeling system, the decision support system, the insulin delivery system, the insulin monitoring system, or the automated control system; or
the one or more of a predictive modeling system, the decision support system, the insulin delivery system, the insulin monitoring system, or the automated control system.

6. The system of claim 1, wherein instructions cause the processor to:
calculate a Euclidean distance between one or more newly received glucose measurement and one or more centroid as the similarity metric.

7. The system of claim 1, comprising plural clusters generated by:
generating an array of glucose measurements for each time range, a plurality of arrays forming a glycemic profile vector;
assigning a weight to an array; and
applying an iterative hierarchical clustering technique that varies a weight until one or more cluster is generated that approximates one or more glycemic profile vector; and
defining a cluster of the plural clusters by a cluster's centroid.

8. The system of claim 7, wherein:
the iterative hierarchical clustering technique computes an R² value by linear regression for an array and varies a weight to maximize the R² value.

9. The system of claim 1, wherein:
the plural time ranges includes five time ranges.

10. The system of claim 1, wherein:
the plural time ranges includes:
Level 2 hypoglycemia below glucose measurement-1;
Level 1 hypoglycemia within a range from glucose measurement-2 and glucose measurement-3;
Target Range (TIR) within a range from glucose measurement-4 and glucose measurement-5;
Level 1 hyperglycemia within a range from glucose measurement-6 and glucose measurement-7; and
Level 2 hyperglycemia above glucose measurement-8.

11. The system of claim 10, wherein:
glucose measurement-1 is 54 mg/dl;
glucose measurement-2 is 54 mg/dl;
glucose measurement-3 is 70 mg/dL;
glucose measurement-4 is 70 mg/dL;
glucose measurement-5 is 180 mg/dL;
glucose measurement-6 is 180 mg/dL;
glucose measurement-7 is 250 mg/dL; and
glucose measurement-8 is 250 mg/dL.

12. The system of claim 1, wherein the glucose measurements include plural glucose profiles for an individual, each glucose profile including plural glucose measurements obtained for a predetermined time period, wherein instructions cause the processor to:
compile the plural glucose profiles into a single glucose measurement time series for an individual; and
classify one or more glucose profile using one or more cluster to generate a sequence of indices representing a classification of one or more glucose profile in the single glucose measurement time series.

13. The system of claim 12, wherein instructions cause the processor to:
generate, using the sequence of indices, a trace representing glucose variability of the individual.

14. The system of claim 12, wherein instructions cause the processor to:
generate an approximated Ambulatory Glucose Report (AGP) using the sequence of indices.

15. The system of claim 1, wherein:
one or more glucose profile of the multiple glucose profiles is a continuous monitoring glucose (CGM) profile including glucose measurements obtained over a 24-hour time period.

16. The system of claim 12, wherein:
one or more glucose profile of the plural glucose profiles for the individual is a continuous monitoring glucose (CGM) profile including glucose measurements obtained over a 24-hour time period.

17. A method for processing glucose data for efficient glucose database management, the method comprising:
receiving glucose measurements;
converting the glucose measurements into vectorial form;
searching a physical data store by comparing a newly received glucose measurement to a centroid of a cluster using a similarity metric, wherein:
the physical data store contains glucose measurement data and a representation for at least one cluster of the glucose measurement data, wherein the representation approximates a glycemic profile vector for a cluster of multiple glucose profiles segmented by plural time ranges;
classifying the newly received glucose measurement with a cluster having a matched similarity metric based on the comparing; and
ascribing a treatment to the newly received glucose measurement.

18. The method of claim 17, comprising:
calculating a Euclidean distance between one or more newly received glucose measurement and one or more centroid as the similarity metric.

19. The method of claim 16, wherein the physical data store contains plural clusters generated by:
generating an array of glucose measurements for each time range, a plurality of arrays forming a glycemic profile vector;
assigning a weight to an array;
applying an iterative hierarchical clustering technique that varies a weight until one or more cluster is generated that approximates one or more glycemic profile vector; and
defining a cluster of the cluster set by the cluster's centroid.

20. The method of claim 19, comprising:
computing, via the iterative hierarchical clustering technique, an R² value by linear regression for an array and varying a weight to maximize the R² value.
